SEQUENCE LISTING (31 sequences)

SEQ ID NO: 1 - DNA, 12011 bp, Artificial sequence
  vector
aagtggaacagtgggcccagagagaatattcaggccagttatgctttctggcctgtaacaaaggacattaagtaaagacagataaacgtagactaaaacgaggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagctttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattgctattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgttttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactaataaaaaaataattataaaaatatccccgtggataagtggataaccccaagggaagttttttcaggcatcgtgtgtaagcagaatatataagtgctgttccctggtgcttcctcgctcactcgaccgggagggttcgagaaggggggcacccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagctcgagggcttcgccctgtcgctcgactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagcccttcaggccttcgccacctctgacttgagcgtcgatttttgtgatgctcgtcaggggggcggagcctatggaaaaacgccagcaacgcggcctttttacggttcctggccttttgctggccttttgctcacatgcatggccatgattacgaattaattcgagctcggtacccgctcacggtaactgatgccgtatttgcagtaccagcgtacggcccacagaatgatgtcacgctgaaaatgccggcctttgaatgggttcatgtgcagctccatcagcaaaaggggatgataagtttatcaccaccgactatttgcaacagtgccgttgatcgtgctatgatcgactgatgtcatcagcggtggagtgcaatgtcgtgcaatacgaatggcgaaaagccgagctcatcggtcagcttctcaaccttggggttacccccggcggtgtgctgctggtccacagctccttccgtagcgtccggcccctcgaagatgggccacttggactgatcgaggccctgcgtgctgcgctgggtccgggagggacgctcgtcatgccctcgtggtcaggtctggacgacgagccgttcgatcctgccacgtcgcccgttacaccggaccttggagttgtctctgacacattctggcgcctgccaaatgtaaagcgcagcgcccatccatttgcctttgcggcagcggggccacaggcagagcagatcatctctgatccattgcccctgccacctcactcgcctgcaagcccggtcgcccgtgtccatgaactcgatgggcaggtacttctcctcggcgtgggacacgatgccaacacgacgctgcatcttgccgagttgatggcaaaggttccctatggggtgccgagacactgcaccattcttcaggatggcaagttggtacgcgtcgattatctcgagaatgaccactgctgtgagcgctttgccttggcggacaggtggctcaaggagaagagccttcagaaggaaggtccagtcggtcatgcctttgctcggttgatccgctcccgcgacattgtggcgacagccctgggtcaactgggccgagatccgttgatcttcctgcatccgccagaggcgggatgcgaagaatgcgatgccgctcgccagtcgattggctgagctcatgagcggagaacgagatgacgttggaggggcaaggtcgcgctgattgctggggcaacacgtggagcggatcggggattgtctttcttcagctcgctgatgatatgctgacgctcaatgccgtttggcctccgactaacgaaaatcccgcatttggacggctgatccgattggcacggcggacggcgaatggcggagcagacgctcgtccgggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattggggatctccatgcatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattccagagcaccgtcgaagccctgatgaagaattcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgactacgcggagctggtgaagtacatcaccgacgagcaaggcaaattgaaaaaggaagagtatgagtattcaacatttccgtgtcgcccttattccctttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtatccctaggcgctccctgcccgctgtggtgacgaaggaactactagttagcctaactaacgatttcaaggccaaaaaaggggccgaaccgtccgccggctcgacccctcccgctgtgcgctacagcgccgcaagctcccgctcagtggcttcgctcgcttcgtcttcctccttcagcagctccgcccacttgagcgtcacacggtccttcagggggatgtatttcttcgtctcagggtggcgccccggtcgaccatgacggagtcaaggaagaagctcaggaactcccgacgctcgtacacgtcccagcttgcccagatgccccttcggccgtcgggtcttcgccgctgaaccactcagacggaacgcgggtgctgccgttcatcttctcgtcaagctcagcgagtcgggtcgtgcactccgcttcgtaggaccggtattgcagcaccgttgagcgccacgtttccagctcttcacgcccgacgtacagaccgggcttacggtccgcctgaaggtccttgatggagcgccgcacgttgtctaggtgcgcctgttgttcgcgccgctcatcggccacccccgctaggtcgtgctgaagggcgaagcgctccgcagcggcggcaatccatgcctgatcgtgttcatcttccatgtcggctgtgcggagccgtgcccacaccttcgaagcaacgaactcgtccagttcgctgcgcttgaccgacaagccgccgtgccccttcgggttggcgcacatgtaattgccttcggcaaggtcgccgttgcgcttacggccaccctgggactgacccattgagccgccgcagattcggcaccccaggaaacgccacccgctcagaagcgtcggttcgacttcggccccaggcttccggtcgctgagattcttcccggaacgcttctcttgaagctcaagccacttcgagccgctgagaatgcccgtgtggggcgttagcggcttgccgccggggtcgcgccgtatgacgttgatgtgcgccttaccgtgcttcacacgctcgaatgcgaaaccgccgattgcgggatggttgagaatccatcggaccgtttgagcgcgccacatgatcggcttttcagcgccgttcaggcgacgtgccttgacggacgcaagacgcttttccgtggcgcgtctctcagccattccgggcgacgggatcttttccttctcgaaggtcgttgcaatggcgttgtcggacacgccttcgaacgacattttcgccatgcgctcaactagctcgacgtgatccgggttgtcttcgtccggctcaagaacggagatcacgagattatcgaccttcttgcgcacggcgcgcattccgaacggggcggaagacgagtgaacgccacccagcgcggcaatctcgtctttcgcacccttcaggcgctccgccttcaggtcactgtcctgtttcgcaagggcagcgatcagcgcgaaaatggcgacgccgataggggtagacgtgtcaaggaacggctcaagaaccgacatgaagcgcacgccgtgcttcttcaattcgttgtcgatttcgagcgcgtcatgggcgcccttgcgagtgagccgggaaagctcattcacaacgacaacgtcaccttcgccggcgcggacttcgcccatcatgcgctcgaagtcggcacgggtcacgttcgggtcccagccggagcgcccgacgtccacgaactcacctgccacgacccagcgcgcacccccctgggcgttgcgagacgcgaccaacgcacgaccggccgctagctgtgcttcggtcgacacgtctgagccgtcggaccggcccttagactgacgcgcgtacaggaagacgcgaacagtgtctagaaggtgctcagggacgtcgggagcgatgaagggcgacatgcctagatcttggctcatgggcgctgacctgcgctttcttttgggaacattggtggtgctgagtagtttcccatggatcactgtccagagacaacaacccagcacccgaaacgtctgaagccccgtccggcgccacctagggatactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgaggcctatggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataat

SEQ ID NO: 2 - DNA, 297 bp, Escherichia coli
acagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactaataaaaaaataattataaaaatatccccgtggataagtggataaccccaagggaagttttttcaggcatcgtgtgtaagcagaatatataagtgctgttccctggtgcttcctcgctcactcga

SEQ ID NO: 3 - DNA, 744 bp, Escherichia coli
tcgagggcttcgccctgtcgctcgactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatga

SEQ ID NO: 4 - DNA, 3000 bp, Escherichia coli
ggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataataagtggaacagtgggcccagagagaatattcaggccagttatgctttctggcctgtaacaaaggacattaagtaaagacagataaacgtagactaaaacgaggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagctttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattgctattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgttttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttcct

SEQ ID NO: 5 - DNA, 704 bp, Escherichia coli
tcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgactacgcggagctggtgaagtacatcaccgacgagcaaggcaa

SEQ ID NO: 6 - DNA, 73 bp, Streptomyces coelicolor
gcccgctgccgtccttgaccaggtttttgacgaaagtgatccagatgatccagctccacaccccgaacgcgag

SEQ ID NO: 7 - DNA, 69 bp, Artificial sequence
  Bacteriophage phi beta T1 attP site
gtttcgggtgctgggttgttgtctctggacagtgatccatgggaaactactcagcaccaccaatgttcc

SEQ ID NO: 8 - DNA, 5719 bp, Artificial sequence
  vector
ggttactccgttctacaggttacgacgacatgtcaatacttgcccttgacaggcattgatggaatcgtagtctcacgctgatagtctgatcgacaatacaagtgggaccgtggtcccagaccgataatcagaccgacaacacgagtgggatcgtggtcccagactaataatcagaccgacgatacgagtgggaccgtggtcccagactaataatcagaccgacgatacgagtgggaccgtggttccagactaataatcagaccgacgatacgagtgggaccgtggtcccagactaataataagtggaacagtgggcccagagagaatattcaggccagttatgctttctggcctgtaacaaaggacattaagtaaagacagataaacgtagactaaaacgaggtcgcatcagggtgctggcttttcaagttccttaagaatggcctcaattttctctatacactcagttggaacacgagacctgtccaggttaagcaccattttatcgcccttatacaatactgtcgctccaggagcaaactgatgtcgtgagcttaaactagttcttgatgcagatgacgttttaagcacagaagttaaaagagtgataacttcttcagcttcaaatatcaccccagctttttttctgctcatgaaggttagatgcctgctgcttaagtaattcctctttatctgtaaaggctttttgaagtgcatcacctgaccgggcagatagttcaccggggtgagaaaaaagagcaacaactgatttaggcaatttggcggtgttgatacagcgggtaataatcttacgtgaaatattttccgcatcagccagcgcagaaatatttccagcaaattcattctgcaatcggcttgcataacgctgaccacgttcataagcacttgttgggcgataatcgttacccaatctggataatgcagccatctgctcatcatccagctcgccaaccagaacacgataatcactttcggtaagtgcagcagctttacgacggcgactcccatcggcaatttctatgacaccagatactcttcgaccgaacgccggtgtctgttgaccagtcagtagaaaagaagggatgagatcatccagtgcgtcctcagtaagcagctcctggtcacgttcattacctgaccatacccgagaggtcttctcaacactatcaccccggagcacttcaagagtaaacttcacatcccgaccacatacaggcaaagtaatggcattaccgcgagccattactcctacgcgcgcaattaacgaatccaccatcggggcagctggtgtcgataacgaagtatcttcaaccggttgagtattgagcgtatgttttggaataacaggcgcacgcttcattatctaatctcccagcgtggtttaatcagacgatcgaaaatttcattgcagacaggttcccaaatagaaagagcatttctccaggcaccagttgaagagcgttgatcaatggcctgttcaaaaacagttctcatccggatctgacctttaccaacttcatccgtttcacgtacaacattttttagaaccatgcttccccaggcatcccgaatttgctcctccatccacggggactgagagccattgctattgctgtatttggtaagcaaaatacgtacatcaggctcgaaccctttaagatcaacgttcttgagcagatcacgaagcatatcgaaaaactgcagtgcggaggtgtagtcaaacaactcagcaggcgtgggaacaatcagcacatcagcagcacatacgacattaatcgtgccgatacccaggttaggcgcgctgtcaataactatgacatcatagtcatgagcaacagtttcaatggccagtcggagcatcaggtgtggatcggtgggcagtttaccttcatcaaatttgcccattaactcagtttcaatacggtgcagagccagacaggaaggaataatgtcaagccccggccagcaagtgggctttattgcataagtgacatcgtccttttccccaagatagaaaggcaggagagtgtcttctgcatgaatatgaagatctggtacccatccgtgatacattgaggctgttccctgggggtcgttaccttccacgagcaaaacacgtagccccttcagagccagatcctgagcaagatgaacagaaactgaggttttgtaaacgccacctttatgggcagcaaccccgatcaccggtggaaatacgtcttcagcacgtcgcaatcgcgtaccaaacacatcacgcatatgattaatttgttcaattgtataaccaacacgttgctcaacccgtcctcgaatttccatatccgggtgcggtagtcgccctgctttctcggcatctctgatagcctgagaagaaaccccaactaaatccgctgcttcacctattctccagcgccgggttattttcctcgcttccgggctgtcatcattaaactgtgcaatggcgatagccttcgtcatttcatgaccagcgtttatgcactggttaagtgtttccatgagtttcattctgaacatcctttaatcattgctttgcgttttttttattaaatcttgcaatttactgcaaagcaacaacaaaatcgcaaagtcatcaaaaaaccgcaaagttgtttaaaataagagcaacactacaaaaggagataagaagagcacatacctcagtcacttattatcactagcgctcgccgcagccgtgtaaccgagcatagcgagcgaactggcgaggaagcaaagaagaactgttctgtcagatagctcttacgctcagcgcaagaagaaatatccaccgtgggaaaaactccaggtagaggtacacacgcggatagccaattcagagtaataaactgtgataatcaaccctcatcaatgatgacgaactaacccccgatatcaggtcacatgacgaagggaaagagaaggaaatcaactgtgacaaactgccctcaaatttggcttccttaaaaattacagttcaaaaagtatgagaaaatccatgcaggctgaaggaaacagcaaaactgtgacaaattaccctcagtaggtcagaacaaatgtgacgaaccaccctcaaatctgtgacagataaccctcagactatcctgtcgtcatggaagtgatatcgcggaaggaaaatacgatatgagtcgtctggcggcctttctttttctcaatgtatgagaggcgcattggagttctgctgttgatctcattaacacagacctgcaggaagcggcggcggaagtcaggcatacgctggtaactttgaggcagctggtaacgctctatgatccagtcgattttcagagagacgatgcctgagccatccggcttacgatactgacacagggattcgtataaacgcatggcatacggattggtgatttcttttgtttcactaagccgaaactgcgtaaaccggttctgtaacccgataaagaagggaatgagatatgggttgatatgtacactgtaaagccctctggatggactgtgcgcacgtttgataaaccaaggaaaagattcatagcctttttcatcgccggcatcctcttcagggcgataaaaaaccacttccttccccgcgaaactcttcaatgcctgccgtatatccttactggcttccgcagaggtcaatccgaatatttcagcatatttagcaacatggatctcgcagataccgtcatgttcctgtagggtgccatcagattttctgatctggtcaacgaacagatacagcatacgtttttgatcccgggagagactatatgccgcctcagtgaggtcgtttgactggacgattcgcgggctatttttacgtttcttgtgattgataaccgctgtttccgccatgacagatccatgtgaagtgtgacaagtttttagattgtcacactaaataaaaaagagtcaataagcagggataactttgtgaaaaaacagcttcttctgagggcaatttgtcacagggttaagggcaatttgtcacagacaggactgtcatttgagggtgatttgtcacactgaaagggcaatttgtcacaacaccttctctagaaccagcatggataaaggcctacaaggcgctctaaaaaagaagatctaaaaactaataaaaaaataattataaaaatatccccgtggataagtggataaccccaagggaagttttttcaggcatcgtgtgtaagcagaatatataagtgctgttccctggtgcttcctcgctcactcgaccgggagggttcgagaaggggggcacccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccggccgcggtgtctcgcacacggcttcgacggcgtttctggcgcgtttgcagggccatagacggccgccagcccagcggcgagggcaaccagctcgagggcttcgccctgtcgctcgactgcggcgagcactactggctgtaaaaggacagaccacatcatggttctgtgttcattaggttgttctgtccattgctgacataatccgctccacttcaacgtaacaccgcacgaagatttctattgttcctgaaggcatattcaaatcgttttcgttaccgcttgcaggcatcatgacagaacactacttcctataaacgctacacaggctcctgagattaataatgcggatctctacgataatgggagattttcccgactgtttcgttcgcttctcagtggataacagccagcttctctgtttaacagacaaaaacagcatatccactcagttccacatttccatataaaggccaaggcatttattctcaggataattgtttcagcatcgcaaccgcatcagactccggcatcgcaaactgcacccggtgccgggcagccacatccagcgcaaaaaccttcgtgtagacttccgttgaactgatggacttatgtcccatcaggctttgcagaactttcagcggtataccggcatacagcatgtgcatcgcataggaatggcggaacgtatgtggtgtgaccggaacagagaacgtcacaccgtcagcagcagcggcggcaaccgcctccccaatccaggtcctgaccgttctgtccgtcacttcccagatccgcgctttctctgtccttcctgtgcgacggttacgccgctccatgagcttatcgcgaataaatacctgtgacggaagatcacttcgcagaataaataaatcctggtgtccctgttgataccgggaagccct

SEQ ID NO: 9 - DNA, 21 bp, Artificial sequence
  primer
agggcttcccggtatcaacag

SEQ ID NO: 10 - DNA, 23 bp, Artificial sequence
  primer
ggttactccgttctacaggttac

SEQ ID NO: 11 - DNA, 28 bp, Artificial sequence
  primer
tcaggccttcgccacctctgacttgagc

SEQ ID NO: 12 - DNA, 28 bp, Artificial sequence
  primer
ataggcctcagtgaggcacctatctcag

SEQ ID NO: 13 - DNA, 34 bp, Artificial sequence
  primer
ctgaattccgacgtcgacgtgtactacctgaccc

SEQ ID NO: 14 - DNA, 34 bp, Artificial sequence
  primer
gaaagcttacgcgatagcgattgccgttgtcgtc

SEQ ID NO: 15 - DNA, 30 bp, Artificial sequence
  primer
cagttcggcgggccggacgtactgggcctc

SEQ ID NO: 16 - DNA, 30 bp, Artificial sequence
  primer
cggcgagggcttccggtccgccgtggcagt

SEQ ID NO: 17 - DNA, 777 bp, Artificial sequence
  plasmid
gtgcaatacgaatggcgaaaagccgagctcatcggtcagcttctcaaccttggggttacccccggcggtgtgctgctggtccacagctccttccgtagcgtccggcccctcgaagatgggccacttggactgatcgaggccctgcgtgctgcgctgggtccgggagggacgctcgtcatgccctcgtggtcaggtctggacgacgagccgttcgatcctgccacgtcgcccgttacaccggaccttggagttgtctctgacacattctggcgcctgccaaatgtaaagcgcagcgcccatccatttgcctttgcggcagcggggccacaggcagagcagatcatctctgatccattgcccctgccacctcactcgcctgcaagcccggtcgcccgtgtccatgaactcgatgggcaggtacttctcctcggcgtgggacacgatgccaacacgacgctgcatcttgccgagttgatggcaaaggttccctatggggtgccgagacactgcaccattcttcaggatggcaagttggtacgcgtcgattatctcgagaatgaccactgctgtgagcgctttgccttggcggacaggtggctcaaggagaagagccttcagaaggaaggtccagtcggtcatgcctttgctcggttgatccgctcccgcgacattgtggcgacagccctgggtcaactgggccgagatccgttgatcttcctgcatccgccagaggcgggatgcgaagaatgcgatgccgctcgccagtcgattggctga

SEQ ID NO: 18 - DNA, 1785 bp, Artificial sequence
  Bacteriophage phi beta T1 attP integrase gene
atgtcgcccttcatcgctcccgacgtccctgagcaccttctagacactgttcgcgtcttcctgtacgcgcgtcagtctaagggccggtccgacggctcagacgtgtcgaccgaagcacagctagcggccggtcgtgcgttggtcgcgtctcgcaacgcccaggggggtgcgcgctgggtcgtggcaggtgagttcgtggacgtcgggcgctccggctgggacccgaacgtgacccgtgccgacttcgagcgcatgatgggcgaagtccgcgccggcgaaggtgacgttgtcgttgtgaatgagctttcccggctcactcgcaagggcgcccatgacgcgctcgaaatcgacaacgaattgaagaagcacggcgtgcgcttcatgtcggttcttgagccgttccttgacacgtctacccctatcggcgtcgccattttcgcgctgatcgctgcccttgcgaaacaggacagtgacctgaaggcggagcgcctgaagggtgcgaaagacgagattgccgcgctgggtggcgttcactcgtcttccgccccgttcggaatgcgcgccgtgcgcaagaaggtcgataatctcgtgatctccgttcttgagccggacgaagacaacccggatcacgtcgagctagttgagcgcatggcgaaaatgtcgttcgaaggcgtgtccgacaacgccattgcaacgaccttcgagaaggaaaagatcccgtcgcccggaatggctgagagacgcgccacggaaaagcgtcttgcgtccgtcaaggcacgtcgcctgaacggcgctgaaaagccgatcatgtggcgcgctcaaacggtccgatggattctcaaccatcccgcaatcggcggtttcgcattcgagcgtgtgaagcacggtaaggcgcacatcaacgtcatacggcgcgaccccggcggcaagccgctaacgccccacacgggcattctcagcggctcgaagtggcttgagcttcaagagaagcgttccgggaagaatctcagcgaccggaagcctggggccgaagtcgaaccgacgcttctgagcgggtggcgtttcctggggtgccgaatctgcggcggctcaatgggtcagtcccagggtggccgtaagcgcaacggcgaccttgccgaaggcaattacatgtgcgccaacccgaaggggcacggcggcttgtcggtcaagcgcagcgaactggacgagttcgttgcttcgaaggtgtgggcacggctccgcacagccgacatggaagatgaacacgatcaggcatggattgccgccgctgcggagcgcttcgcccttcagcacgacctagcgggggtggccgatgagcggcgcgaacaacaggcgcacctagacaacgtgcggcgctccatcaaggaccttcaggcggaccgtaagcccggtctgtacgtcgggcgtgaagagctggaaacgtggcgctcaacggtgctgcaataccggtcctacgaagcggagtgcacgacccgactcgctgagcttgacgagaagatgaacggcagcacccgcgttccgtctgagtggttcagcggcgaagacccgacggccgaagggggcatctgggcaagctgggacgtgtacgagcgtcgggagttcctgagcttcttccttgactccgtcatggtcgaccgggggcgccaccctgagacgaagaaatacatccccctgaaggaccgtgtgacgctcaagtgggcggagctgctgaaggaggaagacgaagcgagcgaagccactgagcgggagcttgcggcgctgtag

SEQ ID NO: 19 - DNA, 21 bp, Artificial sequence
  primer
gcgcggaccgagccctacgac

SEQ ID NO: 20 - DNA, 21 bp, Artificial sequence
  primer
cccccggccctccagcagatg

SEQ ID NO: 21 - DNA, 19 bp, Artificial sequence
  primer
ggttaccttgttacgactt

SEQ ID NO: 22 - DNA, 20 bp, Artificial sequence
  primer
agagtttgatcctggctcag

SEQ ID NO: 23 - DNA, 29 bp, Artificial sequence
  primer
gctctagagtggggaattcaggcgcaccc

SEQ ID NO: 24 - DNA, 30 bp, Artificial sequence
  primer
agcaagcttggggactccggtggagccgga

SEQ ID NO: 25 - DNA, 19 bp, Artificial sequence
  primer
ttgccttgctcgtcggtga

SEQ ID NO: 26 - DNA, 21 bp, Artificial sequence
  primer
cgcacgatatacaggatttgc

SEQ ID NO: 27 - DNA, 22 bp, Artificial sequence
  primer
ggggaggatctgaccgacgcgg

SEQ ID NO: 28 - DNA, 23 bp, Artificial sequence
  primer
cgttggcgtggacccatgtggcg

SEQ ID NO: 29 - DNA, 22 bp, Artificial sequence
  primer
agcccgacccgagcacgcgccg

SEQ ID NO: 30 - DNA, 25 bp, Artificial sequence
  primer
cgggcgcacagctcgcgcagatcgt

SEQ ID NO: 31 - DNA, 94395 bp, Artificial sequence
  plasmid
aattgagctccgggatcgctgccttggaccgcgcggaaaatgaccactcggcgcggcgtggcggagctatcggcgatatcgacgccgagcacaccatcgatgccggtcatacggcccgcgtcgagggcgagcgtcggcggtcggccgagcccaccgtattcgagtccctcgactctcccggctccagcgccgctacgggattcacgctcgaagaaacacttcgcattcgatccctttctcgggtttgccgccgaatgtaatcccggccgtactgccgtttaagaagcgtttacgccatcggttggcaagcgaaagcgacagcaccagcggaataaccgcgaaaagcaatttccatcagtcgtcggggaaggctgtgttgtggggaattcaggcgcacccaaatcccgtggtctcagcgcggccatgagcaatctcttcgagcggaccaggagaaacgaatccaccggcattgtgccggtcgaccggggccgggagctgagggcgtcgttcgcccagcaacggctgtggtttctggaccagttggaacccggcaacgcctcgtacaatctccccttcgcggtgcgggtgcgcggccgcttggacatctctcatctctcccgggccctctcgctcgtggtcgcccggcacgaggcgctgcgcaccaccttcggcgaggccggcggtcagccggtgcagcggatcgagccccccggccccgtcccggtgcgccttgaagcggtgtccggcggctcggaggaggagcggctggccgaggtccggcggctggccggagccgagatcaccgagcccttcgacctgagcaccgggcccctgctgcgcgccaaggcgctgcgactggacgaacaggaccacgtcctgctgctgacggtgcaccatgtggcgacggacgcctggtcacaaggcattgtggtgcgtgagctgtccgtcgcgtacgcgtcgctcgacgccgggcgcgagcccgtgctgcccccgctgcccgtgcagtacgcggactacgcggagtgggagcgcgactggctgtccggcccgaccctgcgccgccagctggactactggacgaagcggctcgacggcatggcgcccgcgctggagctgcccaccgaccggcccaggccctcggtcgccagccaggaaggcgacgcggtgcgctgggagttgccgccggaactgatccgggcggcccgccggctgggcgccggtgagaacgcgaccctctacatgaccctgctggccgctttccagctggtactgggccggtacgtggacagcgacgacatcacggtgggcacccccgtggccaaccggggccgcgccgaggtcgaggggctcatcgggttcttcgtcaacaccgtggtgctgcggaccgacctgtccggcgaccccaccttccgccaactgctgggccgggtccgcgacacggcggcgggtgccttcgcccatggcgacctgcccttcgagtatctggtggagcaggtgcaccccgagcgggacttgtcgcggaacccgctggtccaggtgctcttccagatgatcaacgtaccggcggagcggctcgagctgcccggcgcgcggaccgagccctacgaccacggcggcatcctcacgcgaatggatctggaggtccatctcgtcgagaccggggacggggttctggggcacatcgtcttcagcaaggccctgttcgacacgagcaccatcgaacggctgctgcaccacgtcaccgtcgtcctccggggcgtcctggccgagccggaccggcgcatctccgagatctcgctgctcgacgaggcggagcgggcgaaggtcctggagaagttcaacacgaccacgggccccgtacccgccggatccctgcccgcgctcttcaccgcccaggccgagcgccgccccgatgcggtggccgtgatcagcggtggtgaccgggtgacctacgccgagctggatcagcgggcgaaccagctcgcccatctgctggagggccggggggtcggccccgagaccctggtcgggctctgcgtcgatcgcggcatcgagatgatcgtggcgatcctcgcgatcctcaagctcggagcggcctatgtgccgatcgatccccaccacccccgagaccgcgtccagttcgtccttgccgactccggggtgaccgtcgccgtcacccagcagcgcttcaccggcctgctcgaaaccccggaggcacccgggacgcccgatgcgtccgggacgtccgggatccgcctcatcctgctcgacgccgagcgcgagccgctcgccgggcagccccggaccccgcccacggcacggcccagcgcccagaacctcgcctatgtcatttacacctccggctccaccggagtccccaagggcatcctcatgcccgccacctgtgtgctcaacctggtggcctggcagaagcgggccctgccgatcggtcccgacgccaagacggcacagttcgccacgctgaccttcgatatctcgttgcaggagatcttctccgcgctgctgtacggcgagacgatcgtcgtccccggcgaggaactgcgcatggaccccgccgagttcgccacatgggtccacgccaacgagatcgaccagctcttcgtcccgaatgtgatgctgcgggcgatctccgaggaggtggatccgcacggcaccgagctggccgcactgcgccacctctcacaggccggcgaacccctctccctccaccacgatctgcgcgagctgtgcgcccgccgccccgagttgcggctgcacaaccactacggtcccagcgaagcccatgtggtgacgtcgtactcgctccccgccgaggtggccgagtggccgctcaccgcacccatcggccgcccgatcggcaacacccgggtgtatgtggtcgaccggcggctccggcccgtcccggtgggggtgccaggtgagctgtgcgtggccggagaggggctggccaggggctatctcggccgcccggatctgaccgcttcccggttcgtggcggacccgttccgcggcgacggatcgcgtatgtaccgctccggcgacctggtgcgctggctgcccgacggcaacctggaattcctcggccggatcgatgaccaggtgaagatacgtggcttccggatcgaaccgggcgagatcgaggcgatcctcgcccggcaccaggacgttctgcacacggccgtgatggtgcgcgaggacacccccggcgacaagaggctggtggcctatgtggtggccgatgccaccgccgcggaccggcacggcgggctgaccgagaccctgcgccggcacgtcgagtccgcggtgcccgaatacatggtgccctccgcgttcgtcctgctggacaccatgcccctgacctccggcggcaagatcgaccggaaggcgctgcccgcccccgatctgcgcaccgtgctcgaggtcggctacgtcgccccacgcacccccgaggaagaggccgtctgccgggtttacgcggatctgctcggcgcggccaaggtcggcatcgacgacgacttcttcgcactgggcggccattccctcatcgccaccagggtggtcgccaggctccggtccgccctcggtatcgccgtaccgctgaagaccgtcttccagcagcgcaccccccgagagctggcggccacgctcaccgccgcggcccgctccggtcccgaacccgagctgccgccgctggttcccacgcggcgcgaccagcccgtccccctcaccttcgcacagcagcagacggacctcttcttcgacgatgtcctgaacgccgggcactggaacatccccatggcggtgcgggtgtcgggcgaactggacctcgactgcctgcggcgggcgatggacctgctgatcgaccgccacgaggccctgcgcaccaccttcgtcagggaagccgacggatacgtccaggtgatccggccgagcgcgccggtccaggtggaggtggccgagacgcacgacgagaccgaagcctcggtactggccggccaggaggccgcccgccccttcgacctcacacgcggcccgctggcgagactgcgcgtgctgcggctgtcccagtccgaccatgtgctggtgctcaccctgcaccacctggtcaccgacggctggtcccagggagtgctggtccgagatctgtccatcgtgtacgcggcactgctgcacggcaccgaacccgatctgccacccgcacccgtccagtacgccgatgtcgcgagctgggagcggaagtggttgcgcggtccgctgctgcaacgccaactcgagttctggaagcggcatttcgagggcatgacccccgccgaactgcccaccgaccggccccgcgccgcgtcggcccgctacgagagtgacatcttccactggcgactgccgacggacgccgtcgagaccgcccgacggctgggcgaatcgtgcaacgccaccttgtacatgacgctgctgaccgccctgaaggtggtcatgtccgcccgctcggacaaccaggacgtcctcgtcggcgtgcccacggccaaccgtggccgggacgaactggagaacacggtgggcctcgtctccaagatgctcgcgctgcgcaccgaagtgtccggtgccacggacttcggcacactgctggccacggtgcgcgatgcgatgtccgacgcccatacacaccaggacgtgcccttcgtgtccgttctcaagcacatcggtgaccacaccgccggccccgccggtgacaccgccggcggccgggccgggacgcggctgtcggacgatccgccagtgaaggtgatctttcagatcgtcaacaccccgccgcggccactccggctcaccggactgacggccgagccgttcccgatgacccacccgccggtcacggtcaacgtggacatggagatcgacctgtacgagagcgcggaggacggcggcctcgccggcaccgtgctgttcagcaagtccctcttcgaccgtgccacgatcgagcggttctgcgacgacgtggtggcggtcgtctccgcggccgccgcggatcccggacggccggtctcacaggtgtggcagggccggggccgcgaccagtgaacgatcccgccccgaggaaacgcatggaaccggatgaggccgtcgccgttgtcggaatgtcctgccgctttccgcaggcacccgatcccgaggcgttctggcggctgctgagcgagggcatctcggccatcggtgaggtgcccgcggggcggtggaccgacgaccagcccacgccgtccgggaccgacgagcggtccacgccgcccgccatccgccgcggcggcttcatcgacgacgtcgaccgcttcgaccccgcgttcttcggcatctcaccacgggaagccgcggcgatggacccccagcagcggctgatgctcgagctggcctgggaagggctggaggacgcgggcatcgtgcccgccaccctgcggggcgccaccgtcggagcgttcatcggcgccgggtccgacgactacgcctcgctgatccgcgcccgcggccgttcacaccacacgctgaccggcacccagcggggcatgatcgcgaaccggctctcccatgtgttcggcctgagcggcccgagcgtgaccgtggacgcggcccaggcatcctccctggtcgcggtacacatggccgtggagagcgtgcgccgcggcgagtcacggctcgcgctggcgggcggggtcaacctgaacctctccgcggagaccgccgccgatatcgcggcgttcggcgcactgtccccggacggccgctgcttcaccttcgacgcacgcgccaatggctatgtgcggggcgagggcggcggactcgtcgtcctgaaaccgctctccgacgctctcgccgacggcgacaccgtctactgcgtgatcgagggcagcgcggtcaacaacgacggcggcggtgcatcgctcaccgcacccgacccggacggccagcgacgggtgctccgactcgcccagcggcgggccgcgatctcccccgaggccgttcagtacgtggagctgcacggcaccggaacggcactcggcgacccggcggaagcggcggccctgggcgccgtcttcggccggagcggagcgaggccggtgcagctggggtcggtgaagaccaacatcggccacctcgaagccgccgccggtatcgccggacttctgaagaccgcactggccatccaccaccggcagctgccggccggcctcaattaccgcacgccgaatccccgtatccccatgggcgaactcaacctggagatgcgcctcgcaccgggggagtggccgaagccggacgaccgcctggtcgccggtgtcagctctttcgggatgggcggcaccaactgccatgtcctgctcgccgaaccactcgtcggcgtcccctcccacgcctccgcgcatgcccctgagcccgactccctccccagctcgatcccggccccggtcccggtcccggtcccggtcccggccccggtcccggtcccggccccggccccggccccggccccggtcccggtccccgtcccgcttccgttgtccggggtgtccgctgccgcgcttcgcggccaggcgatgcggctacggccgtatctggagcgatcgccgaacctcaccgacctctccttctccctcgccaccgcacgaacctccttcgaccaccgtgcggtgctgatcaccgggcaggcggccgacgcggcacacggcctggacgcgctcgtcgaaggcggcacggtggcgggtttggtgacgggcacggcgagggcggcgggaaagctcgccttcgccttcgccggccagggctcgcagcgtctcggcatgggacgtgaactcggggccgtcttccccgtctttgcccaggctcttgacgaagtgtgcacggcgctggacgcacacctggaccggccgcttcgggacgtgatccacggtgacgacgccgaaccgctcaaccggacggtgtacgcccaggccggactcttcgcggtggaggtggcgctgttccggctgctggaggacttcggcctcgtaccggacctgctgatcggccactccctcggcgaggtgagcgccgcccatgtcgccggtgtgctgtccttggcggacgccgccaccttcgtcgccgcccgtgggcggctgatgcaggccgtgacggagccgggcgccatggtgtcgctcgaagccaccgaggacgaggtcacccggacgctcatggcgggcggggcatcggacgacggtgcccgggtgtgcgtggcggcggtcaacggccccaccgccacggtgatctcgggggacgagcgcgccgtactcgacctggcggtggagtgggccggtcgcggacgcaagacgaagcggctccggacgagccacgccttccattcgccccatctggaccccgtactggacgagcttcggcacatcgccgagagcctcacgtaccgggcgccccggatcccgctggtgtcgaatgtgaccggccgacgtgccacggcggaagagctgtgttctccggagtactgggtccggcatgtccgccggaccgtacggttcctggacggcgtccgctgtctggaggacgaaggcgtcaccaccatcctggaactgggcccggacaaggcgctcaccaccctggcccgcgactgcctgaccgggcccgggacgctggtgggcacccttcgtcgcgaccggcccgagccgcaggccctggtcaccgcgctggccgagctgtatgtctcgggtgtcgaagtggcatggagcccgctggtgtccggtgggcggcggattccactgcccacgtacgccttccagcggcagcggtactggttctccgctcccgggcccgagagcggaaccacgcctggccatggggtcacatccgggcgcgagcgcacggacaccggcctgagcggcgacgaggcgcccgacaccggcccgagcggcggcgagacgcttggcatggtccgggcgcacgcggccgtcgtgctcggatacgcgtcggcaaccgccatcggcgccgagcacaccttcaagcaactcgggttcgactcgatcaccgccgtcgaactgtgcgaacggctcggtgcggcgaccgcgcttccgctgcccggcaccttgctgttcgactatccgacgcccgccgcgctcgccgagcatctgcaccgcaggctccacggccggacggatgagcaggccgcgcccgcgaccgtgccaacacctgacggcggcgatccggtggtgatcgtggggatgggctgccggttccccggccgggcccactcgccggaggacctgtggcggatcgtggccgacggtgaggacgccatctccggctttccgtccgaccggggctgggacctcgctggtctctaccaccccgaccccgaccaccccggcacgtcatacgcacgcgacggcggattcctctacgacgcggccgagttcgacgcggggttcttcgggatctcaccgcgtgaggccgaggcgatggacccgcagcagcggctgctgctggagacatcgtgggaggcgttggaacgggcgggtatccccgcggaacacatcaagggcagtagcacgggcgtgttcatcggcgcctcgtcggtcggctacgcggcggacgccggagaggcggccgagggctaccagctgaccggcactgccgcgagcgtggcctcgggcagggtgtcctacaccctgggcctcgaaggcccggcggtcaccgtggacacggcatgctcgtcctcgctggtggcattgcacctggccgtacagtcgctgagggcgggcgagtgctcactggcattggcgggcggtgtgaccgtgatggccacaccggcgatgttcgtggagttctcccgtcagcgggggctggccatggacggtcggtgcaaggcgttcgcggcggcggcggacggcacggggtgggccgaaggcgtcggggtgctggtggtcgagcggttgtcggacgccgagcgcaatgggcatcgggtgttggcggtggtgcgtggttctgcggtgaatcaggatggtgcgtcgaatggtttgacggcgccgaatggtccgtcgcagcagcgggtgatccggcaggcgttggcgagtgcgggtcttgtggcgtcggatgtggatgcggtggaggcgcatggtacgggtacgacgctcggtgatccgattgaggcgcaggcgttgttggccacgtacggtcagggtcgggatgcggatcggccgttgtggttggggtcggtgaagtcgaacatcggtcatacgcaggcggccgcgggtgtggctggtgtgatcaagatggtgatggccatgcggcacggggtgctgccgcgaacgctgcacgtggatgagccgtcgacccacgtcgactggtccggcggccgggtagagctgctcaccgggacaacgccatggcccacgacgggtggccttcgccgagcgggcgtctcctcgttcggtgtgagtggcaccaacgctcacgtcatcctggagcaggtcccggagacggcccggccgaccgggcccatcggggaagacgacggcgaagcggcgcccgtcgcctgggtgttgtcgggacagggcgagactgggctgcgggcccaggccgagcggctgtgcgccttcatggcggccgatacccgccccaccccggcggaagtgggatggtcactggcatcgacacgtgcgacgttgtcgcaccgcgcggtggtcgtgggtgctggacgcgacgagttgttgcgtggtgtgaatgcggtggcgaacgggacacccgtgccgggagtggtacggggcaccggagcctccggggacgtggtgttcgtcttcccggggcaggggtcgcagtgggttgggatggcgttggagttggtggagtcgtcgccggtgttcgcgcggcggttgggtgattgtgcggatgcgttggcgccgtttgtggagtggtcgttgttcgatgtgttgggtgatgaggtggcgatcggtcgggttgatgtggtgcagccggtgttgtgggcggtgatggtgtcgttggcggagttgtggcgttcgtttggtgtggtgccgtcggcggtggtggggcattcgcagggtgagatcgcggcggcgtgtgtggcgggtgcgttgactttggaggatggggcgcgtgtggtggccttgcggagcagggcgttgctggctctgtcgggtcggggcggcatggtgtccgtaccggtgtccgccgatcggctccgtgaccgtgtggggttgtcggtggcggcggtgaatggtccggcgtcgacggtcgtgtccggggcggttgaggtgctggaggcggtgctggcggagttcccggaggccaaacggattccggtggattatgcctcgcattcggtgcaggtggaggggatccgggaggggctggcggaggcgttggcgccggttcggccgcgtacgggtcaggtgccgttctattcgacggtgaccggccggctgatggacaccatcgagttggacgcggagtactggtacaggaacctgcgcgagacggtggagttccagagcaccgtcgaacacctcatgcgccagggtcatacggtgtttgtcgaggccagcccgcatccggtgctgaccatcggcgtccaggacaccgccgacaccaccgacactgacatcgtcgtcaccggatcgctgcgccgcgatgatggcactgtccagcggtttctgacctccctggccgagctccacgtgcgcggtgtccggatcgactggggcccgctcttcgccggtgtctcgcccgttgagctgccgacgtacgccttccaacgggaacggttctggcttggggcggacatcgccgagtccgccgtggacacgtggcgataccagatctcctggaagccgctgccggacatggaccccccggccctctccggcacctggctggccgtggtccccgaaggggacgagtgggccatggcgggcgcacgggcgctgatcgagtcgggcacggccagcgtccgtaccctccaggtgacctgcgacgcggaccgccggaccctggccgggccgctgacggatgtggcgggatccgaagacatcgccggtgtcgtctcgttcctggccgccgacgaagttccgcatccggcccaccccgcgctgtcccgggggatggcgcacacggtcgagctgctgtgctcgctcaccactgccgatgtcgaggccccgctgtggtgtgtcacccgggcggccgtcacggcactgcccgcggacccggcgccgagccccgcccaggcggcggtatggggattcggacgggtggccgggctggagcgatccgagcggtggggcggcctgatcgacctgcccgtccactgcgacgcacacgtgctgcggcggttcgtcgccgtactcgcgcaggcagccggtgaggaccaggtggcggtgcggccatcggcggccctgggccgacggttggagccggcgcccaggaccggaccggccggcgcatggcgcccgcacggcacggtgctgatcaccggtggcaccggcgtgctgggcgcacatgtggcacggtggctggcgcggtccggcgcggaacacctggtgctgctcagccgccgtggcccgcaggcccctggggcggccgtgctcgacgacgaactgaccgcgctcggcgtacgagtgaccctgacggcctgcgatgtgaccgaccgggccgctctcgccggggtgctggcatcggtgccggacctcaccgccgtggtccatctcgcggggaccgtgcgattcggcaattccatcgacgcggacctcgacgagtacgccggcgtcttcgacgccaaggtcaccggtgccctgcatctggacgagctcctcgaccactcgtcactggaggcgttcgtcctcttctcctcggcagcggccgtctggggcggtgtcggccaggccggttacgcggcggcgaacgccctgctcgacgcggtggcacagcggcgtcgcgcacgcggtctgccggccacttcgatcggctggggcacctggggcggcagcctcgcgcccgaggacgaggagcggctgagccgcatcggcctgcgcccgatgcggccggaggtggccgtcaccgagctgcgccacgtcgtcggatcggccgagccctgcccggccatcgcggacgtcgactgggagaccttcggcccggccttcacggcaggccggcccagccgcctgctcagcgagttgccgcggctgcgaaacacctccggcgccatggcgatgaccggcgaccacgccgcattgcggaggcgactggccggggtgtccgcggccgaccaggcccggacgctggtggacctggtacgtgaacacgcggcggaactcctggggcaccgcggcccggcggcgatcgaccccacggtgccattccggcaactgggcttcgactcgctgacggcggtcgagctgcggacccggctgaacgcggccacgggactgcgcctcccggccaccttgctgttcgaccacccgagctgccgggcggtcgccgatctgctgcgctcggaactgctcggcgaccggccgggctccctcgcggcgtcgtccgccacggaggctgtgcccgccggcgtggtggcctccgacgagccgatcgccatcgtcgcgatgagctgccgcttcccgggaggcatcggaacccccgaggacttgtggcgggtggtcagcgagggccgggacgtgctctccgacttccccgacgaccgcggctgggacgtggacgcgctgtacgacccggacccggaccggcccggcaccagctatgtgcgtaccggtggattcctccacgacgccgcggagttcgacccggaactcttcgggatctccccgcgtgaggcgctggcgatggatccccagcagcggctgctgctggagtcggcgtggcaggtcctggagcgcgccaggatggcgccgacctccctgcgatccagcaggaccggtgtcttcatcggcggctggggccagggctacccctcggcctcggacgaggggtatgccctgaccggcgccgcgaccagcgtgatgtccggtcgtatcgcctacgcgctggggctggagggccccgccctgaccgtggacacggcatgttcgtcctcgctggtggcgctgcatctggcgagcgaggcgctacggcgcggcgagtgctcgctggcgctcgccggcggcgtgacggtgatggcgacgcccagtacctttgtggagttctcgcgccagcgtgggctggccccggacgggcgctgcaagccgttcgccggggcggcggacggcacggggtggggcgagggcgtgggcatgctgctggtggagcggttgtcggatgctgagcggcttgggcatccggtgctggccgttgtctccggctctgcggtgaatcaagacggtgcgtcgaatggtttgacggcgccgaatggtccgtcgcagcagcgggtgatccgtcaggcgttggcgagtgcgggtcttgtggcgtcggatgtggatgcggtggaggcgcacggtacgggtacgacgctcggtgatccgatcgaggcgcaggcgctgctggccacctacggtcaggaccgggatgcggatcggccgttgtggttggggtccctgaagtcgaacatcggtcatacgcaggcggccgcgggtgtggctggtgtgatcaagatggtgatggccatgcggcacggggtgctgccgcgaacgctgcacgtggatgagccgacaccgaaggtggattggtccgccggcgcggtgggactgctcaccgagtcggccgagtggcggcaggagggccgaccgcgccgagccggggtgtcggctttcggggtgagcggcaccaatgcccatgtgatcctggagcaggccccgaagcacgcaccgggggtggcggccgagggcaggaaggggcgcggggagccgccgacggtgccctgggtgctgtcgggcgcgagcgaggcgggtctgcgggcgcagatcgaaggcttgcgggccttcgctgacgacaaccccacgctcgatccggcggatgtgggctggtcgttggcgtccacacgtgcgcttctgccgtatcgcactgtcgtcgtgggcaccgacctcgacgagttgcggcgtgggttggacgcggcggaggtggtgggcgcggccgagccggaccgtggcgccgtgttggtgttcccggggcaggggtcgcagtgggttgggatggcgttggagttggtggagtcgtcgccggtgttcgcggggcggatgcgtgattgtgcggatgcgttggcgccgttcgccgagtggtcgttgttcggtgtgttgggtgatgaggtggcgcttgggcgggttgatgtggtgcagccggtgttgtgggcggtgatggtgtcgttggcggagttgtggcgttcgtttggtgtggtgccgtcggtggtggtggggcattcgcagggtgagatcgcggcggcgtgtgtggccgggggtctgtcgttggaggacggtgcccgtgtggtggccttgcggagcagggcgttgctggctctgtcgggtcggggtgggatggtgtcggttccggtttctgctgaccggctgcggggtcgtgtggggttgtcggtggcggcggtgaatggtccggtgtcgacggtggtgtcgggggctgttgaggtgctggagggggtgctggcggagttcccgggggccaagcggattccggtggattatgcgtcgcattcggtgcaggtggaggggatccgggaggggttggcggaggcgttggcaccggttcggccgcgtacgggtgaggtgccgttctattcgacggtgaccgggcgattgatggacaccgtggggctggatggggagtactggtatcggaatctgcgtgagacggtggagttccagtccgcgatcgaggggctgctggagcttggtcatacggtgttcgtcgaggccagcccgcatccggtgctgaccgtcggcatccaggacaccgccgagaccacggacaccgacatcctcgtcaccggctcgctgcgccgtgacggcggtggccttgcctctttcctcaccgcgctggcccggctgcatgtccggggtgtcgcggtggagtggcgggaggcgttcgccgggctggacgcccacgccgtggacctgccgacctacgcctttcagcgtcggcgcttctgggcggcctccctgcggcagactcccgggacggccgagttcgaccatcccctcctgggcgcggtgctgcccttgcccgattccggcggcggtctgctcacgggcgtgctcacactggccggacagccgtggctggccgaacactcggtggccggtgtggtgttgttcccggggacggggtttgtggagttggtgttgcaggcggggttgcggtgggggtgtggggtggttgaggagttgactttggaggggccgttggtgcttccggagcggggtgaggttgaggttcaggtttcggtgggtggtgtggatggggcggggtgtcggtcggtgtcggtgttttcgtgtcgtgggggtgagtgggttcggcatgcggtgggtgtgcttggggtgggggatggtgtggtgccgggtgtggaggtgtggccgccggtgggtgcggagcgggttggggtggagggggtttatgaggttttggcggagcgggggtatgtgtatgggccggtgttccaggggttgcgggacgcctggcgccggggcgacgaaatcttcgtggaggcggaggtaccggcggaggcgcggggcgatgcggctcgctgtgccatccatcccgcgctgctcgacgcagggctgcacggcgtcggattgggcggcctgatcagcgacgacggccgggcgtacctgccgttctcctggagcggggtcaggctgcacgcggtcggcgcatccgctgtccggatgacgctgacgcccgccggaccggacgcggtgtcgctgagggtgaccgatgaggcgggcgaggcggtgctgacggcggactcccttgtgctccgcccggtcaccgagggacagctcgccgaagccgagatcggcaaccgcgatgtgcttcatcgggtggagtgggtggatgcgggggcgtgttcggtggggtcgttcgtggagtggggtgaggtggctgctggtggggtggtgccggattgtgtggtgttggccggggctgatgtggcgggtgtgttggaggttttgcggacgtgggtggtggaggagcggtttgagggttcgcggttggtggtggtgacgaggggtgcggtgtcggtcggtggtgagggtttggaggatgtgagtggtggtgcggtgtgggggttggtgcggtcggcgcagtcggagcatccggggcggtttgtgctggtggacgccgatgtagatacggatgtggttccggatgtggtggggctgggggagtggcaggtggcggtgcgtgcgggtcgggtgtgggtgccgcgtctggtggatgtggatgtgagtgtgggtggtgctgtggtgcgtgggggcttgggttcgggtgtggcgttggtgacgggtgggacggggttgctgggtgggttggtggcgcgtcatctggtgtcggcgtatggggtgggtgagttggtgttggtgagtcgtcggggggtggctgcgccgggcgtggaggagttggtgggggagttggaggggttgggcgcgcgggtgcgggtggtggcgtgtgatgtggcggatcggggtgcggtggcggagttggtggggtcgatcgaggggttgcgggtggtggtgcacgcggcgggtgtcgtggatgacggggtgatcggttcgttggacgcggagcggttgtgtggggtgatggggccgaaggcgtggggtgcctggcatctgcatgagctgacgcgtgggttggatctgtcggcgttcgtgttgttctcgtcggcggcgggtgtgttgggcaacgcgggccagggcggctacgcggccgcgaatgggttcctggacgcgctggcggttcaccgtcgggggcggggactccccgcggtgtcgatcgcgtggggcttctgggaggaacgcagcgaactgaccgccgacctggccgaggtgcagctgtcgaggatctcccggtccgtaggggccagcatcagcagcgcacaaggactggatctgttcgacgcggcgcttgccgccgacgagccgatggtgctggccacacccctgaacctgcccgcgttgcgggaccaggccgccgcgggcacgttgccctcgatcctgagcggactggtcaccgctcccgtccgcaggacggccggcaccgggcgcactccggccggactgcggcaccaactcgccggggtgacagaggccgaaaggcagcaccagatcatgcgcctggtgcaggaacatgtggccggcgttctgggacatgcctccgcggagttggtcgacgcctcgcggacgttccaggagatcgggttcgactcgctgaccgccgtggaactgcgcaaccggatcagcgccgccaccggcatacggctgcccgccaccgcggtcttcgaccaccccacgcccaggctgctggccgagcgggtgctggccgaggtagggggctccttgccgaccgccgccccgatcgcgccggtgtcggccgtcgatgacgagccgatcgtgatcgtgggcatgagttgccgcttccccggcggcgtcgagtcccccgaggacctgtggcgcctggtccactcggccaccgacgcggtctccgcgctgcccacggaccggggctgggacctggccaccttgtccggtgccaagggcggcgccggtgcctcgtacgcccgggacggcggattcctttacgacgcggctgagttcgacgccggattcttcgggatctcgccgcgcgaggcgaccgcgatggatccgcagcagcggctgctgctggaggcggcctgggaggtgttcgagcgggccggaatcgccccggacacgctcaaaggcagccggacgggcgtcttcacaggcgtgatgtaccacgactacggctcgtggctcaccgatgtcccggaggacgtcgagggctatctgggcacaggcatcgcgggcagtgtggcgtcggggcgactcgcctatacgttcggccttgaggggcctgccctgacggtggacacggcctgctcctcatcactggtggcgctgcatctggcggccgagtcgctgcggcgcggggagtgctcgctggcactcgcgggcggcgtcaccgtactggcgactccgcaggtcttcgtggagttcacacgccagggcggactcgcaccggatggccggtgcaagcccttcgccgctggtgcggatgggacgggctggtcggagggtgttgggctgctgctggtggagcggttgtcggatgccgagcggaacgggcatccggtgctggccgttgtctccggctccgcggtgaatcaagacggtgcgtcgaatggtttgacggcgccgaatggtccgtcgcagcagcgggtgatccgtcaggcgttggcgaacgccgggctcgccgccagggatgtcgatgcggtggaggcgcatggtacggggacgacgctgggtgatccgatcgaggcgcaggcgttgctggccacgtacggtcagggccgggatgtgggtcagccgttgtggttggggtcggtgaagtcgaacatcggtcatacgcaggcggctgcgggtgtggctggtgtgatcaagatggtgatggctatgcggcacggggtgctgccgcgaacgctgcacgtcgatgagccgtcgccgcatgtggattggtctgctggggcggtggagctcctgggggagcacatgggctggccggaggtcgggcggccccgtcgggcgggtgtctcgtcgttcggggcgagtggcaccaacgcccatgtgattcttgagcaggcccccgacatggcgggtgaacctgagcaaaggccggagcgtaacgaactaccggcgattccctgggtgttctccgctggcgacgaggcgggtttgcgggcacaggccgtacggctacgggccttcgcggaccggaatccggatctggatccggtggatgtggggtggtctttggcgactggtcgtgcggggttgtcgcatcgtgcggtggtggtgggtgcgggtcgtggtgagttgttgggggctttggagggtgtgccggtggtgggtgtgccggtggtgggtgggttgggtgtgttgtttgcgggtcaggggtcgcagcggttggggatgggtcgtgggttgtatgaggggtatccggtgttcgctgcggtgtgggatgaggtgtgcgcgcagctggaccagcatttggataggccggtgggtgaggtggtgtggggtgatgatgccgggttggtcggggagacggtgtatgcgcaggcggggttgttcgcgcttgaggtggcgctgtatcggctgatcgcttcgtggggtgtgaggggggattatctgctgggtcattcgattggtgagttggctgcggcgtatgtggcgggtgtgtggtcgttggaggatgcggggagggtggtggtggcgcggggtcgtttgatgcaggcgttgccgtcgggtggtgcgatggttggggtggcggcgtcggagggtgtggtgcggccgctgctgggcgagggtgtggtggttgcggcggtgaatggtcccgagtcggtggtgctgtcgggtgatgaggatgcggttgaggcggttgtggatgtgttggctgggcgtggggtgcggacgcggcggttgcgggtgagtcatgcgtttcattcggctcgtatggacgggatgctggcggagttcggtgaggtgcttcggggggtggagttccgtgccccgagcgtgcccgtggtgtcgaacgtgtccggtgcggtggcgggtgaggagttgtgttcgccggagtattgggtgcggcatgtgcgggagacggtccggttcgccgatgggctggatactctccgtgagctgggtgtgggttcgttcctggagttggggccggacgggacgttgaccgccttggcggatggcgatggtgtgcctgtcttgcgtcgggatcgtccggagcctctgaccgctatggcggctttgggcgggctgtacgtccggggtgtccagatcgactgggatgcggtgttcccgggtgctcggcgggttgatttgccgacgtatgccttccagcgtgagcggttctggttggagccgtcccctgagcggcccacgacgagcgtggttgacgcggcgttctgggatgcggttgagcgtggggatctcggttcgttcggcatcgatgccgagcagccgctcagcaccgccctgcccgccctctcgtcctggcggagggcgcggcaggagcagtcggtgattgatggctggcgttaccggctcggttggatgccgattccggcggtgtccggggaggtgggcctcaccggtacctggctggttgtggtcgagccgggtgcggacggtactgatgtggctgtcgcgttgcggtcggccggggccggtgtcgaggttgtgacgtcggcggagctgagcgctggtccggttgcgggtgtggtgtcgttggtgtcggtcgaggcgacggtgtcgttgctgcacgtccttgtggcggccggggtcgatgcgccgttgtggtgtgtgactcgtggtgcggtctcggtggtcgacggtgacttggtggatcctggccaggcgggagtctggggtctgggccgtgtgatcggtctggagcatccggatcgttggggcgggctgatcgacttgcctggcgaactggacgatcgcgcggggaatgcgctggtaggcatccttgccgggggcaccggtgaggatcaggtggccatccgtgtcaccggcatatggggtgcccggctggtgcgggcgacgccggtcccgatcggtgacgcgggtggtgaggctgcggccgcgtggcgtgggcgtggtaccgcgctggtcaccggtggtacgggggcgctggggcgccaggtggcgcgctggctggtggacagtggtctggagcgggtcgtgctgacgagccgtcgggggggcgaggcgcccggtgccgtcgagctggtggctgagttggggagccgagtgcgtgtcgtggcctgtgatgtcggcgatcgtgaggagcttgcggctcttttggcgatgctcccggatgtgcggaccatcgtgcatgcggcgggtgtcctcgacgacggggtgctcgaatcgctgacgcccgagcggatccgtgaggtgatgcgggccaaggccgacggcgcgcggcatctccacgagttgacccgtgacatcgacctcgacgccttcgtgttgttctcgtcggctgccgggaccgtgggtaatgcgggtcaggggagctatgcggcggccaacgccgtcctggacgggctggcgtggcgtcgccgggccgagggcttggtggccacatcggtggcctggggagcctgggccgacagcggcatgggggctgggcacgcacgggccatggcaccacggctggcgctggcagcccttcagcgagcgttggacgacgacgagaccgcactcatggtcgcggacgtggattggtcgagcttcggctcccggttcaccgccgtacggccgagcccgctgctgagcgaactgctgccccgctccagcgcgccggtggaaccggtcgaggcactcgccacccggttgcggggcatgtcgcggatcgagcgcgatcgggcggtgctggagctggtccgtgcccaagtggcggccgtgctgggacatgcgaagcccgcttcggtcgacccctcgcggaccttccaggaagtcggcttcgactcgctgaccgcggtggagctgcggaaccggctggccactgccaccggcgtaccgttcccggggtcggtcatcttcgactatccgactcccacggcgctcgccgaccatgtccgggcccggttcgttccggacacggacaacgacgaggacgggggcggcgcgacgtccgtgctcgacgagctgaccaggctggaagccgtgctgtccgacctgtccccgagcgacgtggccggtgccgaggtcgccgcgaagatcaagagcctgctgtcccactggggagcggccaccaacagtgacatcgacatggattccgcgacggacgaggagatgttcgacctcctcggcaaggagttcgggatctcgtgaacctgccgtcgagttcgtctccgagtgagtccagcaccgcgttgagagggccgtcctgtggagaatgaagagaaacttcgtcattacctcaaagaggtcacgaaggatctgcggcagacccgccagcgcttgcaggacgtcgaggcgaagagccgcgagcccatcgcgatcgtcggcatgagctgccgtttccccggtggcatcgcaacgccggaagcgctgtgggacctggtgcgcgagggcggcgacgcggtgtcggagttcccggccgaccgcggatgggacacggagggcctctacgaccccgcgggcggctccgggaagtcggtcacccgctacggcggattcctgcgcggcgtcgccgatttcgacgccgcgctcttcgggatctctccccgtgaggcgatcgcgatggacccgcagcagcggctgatgctggagacctcctgggaagcgttcgagcgggccggtgtcaaccgtgacgcggtgcggggcagccggaccggggtgttcatcggcaccaacggccaggactacgcgacactgctcagcgctgcccgggacgatgtgcaaggccacctcggcacgggcagcgcggccagtgtgctctcgggacgggtcgcctacaccttcggtctcgaagggccgacggtcaccgtggacaccgcgtgctcgtcctcactgatcgccctgcacctggccgtccaggcactgcgcaacggcgagtgcgagctggcgctggcgggcggcgtcacggtgatgacgacgacgaacaccttcgtcgagctgtccaagcagggcgggctggcgccggacggccggtccaaggcgttcgcggcggcggcggacggcaccggctggggtgagggcgccgggatgctgctggtggagcggctgtccgacgccgaacggcacggtcaccccgtgctggcggtggtgcgtggcaccgccgccaaccaggacggcgcgtcgaatgggctgaccgcgccgaacgggccctcccagcgccgggtcatccgcgcggcgctgtccaacgcccagctgtccacgggcgatgtcgacgtggtggaggcacacggcaccggcacccggctcggcgacccgatcgaggcacaggccctgctcgacacctacggtcaggaccgggaccggccgctgtggctcggatcggtcaagtcgaacctgggacacacccaggccgccgcgggtgtcgccggggtcatcaagatggtgctcgccatgcgccacggtgtgctgccgcgcaccctgcacgtggatgaaccgaccccgcatgtggactggtccgccggggcggtgcggctgctcaccgagcggaccccgtggccggaggccgaccggccgcgcagggcgggcgtctccgccttcggagtgagcggcaccaacgcccatgtgatcgtggagcaggcatcggaggccgagcccgtcgagccgccccgggccgaaccggtgacggtgccctgggtgctctcgggccagggcgaggccggtctgcgggccttcgcggcccggctcgccgatgtggccaccgaagcgcaccccggcgacctcggatggaccctggccaccacccgctcggcgctgccgcaccgtgcggtggtgatcggatccacaccagaggaactgcggagcggcctcgcggcggtggccgccggagagccggcctcgaacgtggtggagggagtggccggctccgacaccggcgtggtcttcgtcttcccgggacagggctcgcagtgggccggtatggccgtggaactgctggactcctccccggccttcgcccgccggttcgccgaatgcgcccgtgccctggagacacacctcgactggtccatcgaggacgtggtgcgttccgcgcccggtgcgccctcgctcgacctcatcgaggtcgtccagccggtcctgttcaccatgatggtgtccctcgctgagctgtgggcctcctacgggatcactccatcggccgtggtcggccactcccagggcgagatcgcggcggcctgtgtggccggggcgctgtcgctggaggacgcggccaaggtggtggtgttgcgcagccgcctcttcgccgaaacgctggtgggcaacggcgccatcgcctcggtcgccctgcccgcggaacaactggccacccggatcgagccgtggggcgagcgcctcgtggtggccggggtgaacgggcccgcggccgccacggtggccggcgatccccagagcctcgaggagttcgtcgccgcatgcgcggcggacggcgtacgcgcccgcgtcgtgcccgccaccgtggcctcccacggcccgcaggtggaaccgctgcgggaacggctgctcgccctgctggccgacgtggcgccacgccagtccaccgttccgttctactccacggtgaccggcggactcctggacaccaccgaactcgacgcggactactggttctggaacgcccgtaagccgatcgacttcctcggcgcgctccgggcgctgttcgccgacggccaccgcgtcttcgtggagtcgagcacccaccccgccctgaccatgggggtccaggacaccgcggatgcctccggcgagtccgtggaggtcaccggctcgttgcggcgtggcgagggcgggctcgaccagttccactcggccgtggcgcggctgcatgtgcacggcgtacgggtggactggtccgcggccttcggggcggcgcggcgggtggagctgccgacctaccccttccagcgggagcgttactggctgacgccccggcccggccagggtgacgcctccgccctggggctgggtgcgctcgaccaccccctgctgggggccacggtcgtgctgcccgagtccggcggttgcctgctcaccggtcggctgtccctggccggacagccgtggctggccgatcacgccctctccggtgtggtgttgctgccggggacggggtttgtggagttggtgttgcaggcggggttgcggtgggggtgtggggtggttgaggagttgactttggaggggccgttggttcttccggagcggggtgaggttgaggttcaggtttcggtgggtggtgtggatggggccgggtgtcggtcggtgtcggtgttttcgtgtcgtgggggtgagtgggttcggcatgcggtgggtgtgcttggggtgggggatggtgcggtgccggtggcggaggtgtggccgccggtgggtgcggagcgggttggggtggagggggtttatgaggcgttggcggagcgggggtatgcgtacggcccggtgttccaggggctgcgggacgcctggcgccggggagacgaaatcttcgtcgaggtggcggtggcccaggaggcacgggcggacgcggcgcggtgcgcgatccatcccgcgctgctcgacgccgcgctccacggggtgcgattcggtgacttcgtatccgacgacgaccaggcttatgtgccgttctcctggaccggcgtcacgctgcacgcggtcggtgcgacggtcctgcgcgtcacactgtccccggcaggacgcgacgcgatcgccctccgggccacggacaccaccggtgcgccggtcctgtcggcacgctcactggccctgcgaccggtctccgcccagcagttgaacgacacgcgggggagcaggactgacgccctccatcgggtggagtgggtggacgcgtccggaaccgtggcggtggggggtgaggtggcgccgcggactgaggtggtgcgggtcgtctccgagggtccggatgtggtgggtgaggcgtacgggcatgtgcttgaggttctggagcgggtgcaggcgtgggtggcggatgaggacctggcgggtgagcggttggtggtggtgacgcggggcgctgtcgacacgggtgatggtgtggcggacgtggctggggccgcggtgtggggcctggtgcggtccgcgcagtcggagaacccggggcgtctggtgctggtggacaccgatgacctggacggcgtcgacagtctgcttcccgggatgctggctctggatgaggagcaggtgctggtgcggtcgggtgcggtgcgggtgccgcgtctggctcgggtgccggcgccgggtgaggtatcgggagggtttggttccggtgcggtgttggtgacgggtggcactggtgtgctgggcggtctggtgtcacggcatctggtggcgcggcatggggtgagcaggctggtgctgctgtcgcgtcgcggtgcggaggccgaaggtgcggcggagttgcgggaggagctggaggccgcgggcgccgaggtggtgatcgcggcgtgtgatgccgcggatcgtgaggctctggccggggtgttgtcggggttgtcggcggacttcgccttgagcggtgtggtgcatgcggcgggtgtgctggacgacgggttgctcacgtcgttgacgcgtgagcgggtcgagccggtgttgcgggcgaaggtggacgcggcgtggaacctgcatgagctgaccacgggcatggatctgtcggcgtttgtgctgttctcatcggcggcgggtattctgggcaacgcgggccagggcagttatgcggcggcgaacgggttcctggacgcgctggcggctcatcggcgggcgcggggactgcccgcggtgtcgatcgcgtggggcttctgggaagcacgcagcgagctgacccagcacctgtcggccgacgatctggcgcgtgcccacgcggtgccgatgcccacctcccaggcactggatctgttcgacgcgacgctcgccgccgacgagccgatggtgctggccgcacccctgaacccgcaggcatggtcggacgccggccacctgcctcccgtcctgcgcgatctggtccggccgcggatccggcgcgcggcggagacaaccggcgcccccgaatcggcctccgcgctcggacaccggctggccgccgtcgaccgctccgagtgggaccaggtcgtacgcgaactcgtgcgcaatcacatcgcggcggtgctgcgccatgcctccggggagtcggtggacacctcgcggacgttccaggagatcggcttcgactcgctgaccgccgtggaactgcgcaaccggatcagcgccgccaccggcgtacggctgcccgccaccgccgtgttcgactacccgacaccgcaagcgctggccgagtacctgctcgccgaagtcctcgggaaggacagcgccgccgccgcgacacccgtcggaaccgccctcgtcgccgacgatcccatcgtcatcgtcggaatgagctgccgctaccccggcgggatcacctcgccggaagcgctgtgggacctggtgcgctcggacggcgatgccatatccgtcctgccggccgacagaggatgggacctggacggcctctacgacccggatccggaccgcaccggtacgtcgtacgcccgcagcggtggattcgtctacgacgcggccgagttcgacgccgccttcttcgggatctcgccgcgcgaggccgccgccatggacccgcagcagcggctgctactggaaacctcatgggaggcgttcgaacgcgcgggcatccccgccacctccgtcaagggtgagcggatcggcgtgttcaccggggtgatgcaccacgactacctcacccgcctgtcgaccacaccggacgccgttgagggctatctgggcacgggcgcggcagcgggcgtcgcctcgggccgcgtggcctacaccttcggactcgagggcccggcggtcaccgtggacaccgcctgctcgtcgtcgctggtggccctgcacctcgccgtacaggcgctgcgcctcggcgagtgctcgctcgcgctggccggtggtgtgacggtgatgtccacgcccaccgtcttcgtcgagttctcccgccagcgcgggctcgcgccggacggcaggtgtaaggcgttcgcgggagcggcggacggcaccggcttcgccgaaggcatcggcatgctgctggtcgaacggctctcggacgcacggcgcaacggacaccccgtcctggccgtggtgcggggcagtgcggtgaatcaggatggtgcgtcgaatgggttgacggccccgaatggtccgtcgcagcagcgggtgatccggcaggcgctggcgagcgcggggctgtccacggtggatgtggacgcggtggaggcgcacggtacgggtacgacgctgggtgatccgatcgaggcgcaggcgttgctggccacgtacggtcagggccgggattcggaccggccgttgctgctggggtcgatcaagtcgaacatcggtcacactcaggcggccgccggtgtggctggtgtgatcaagatggtgatggcgatgcgccacggcgtgctgccgcagagcctgcacatcgatgagcccactccccacgtcgactggtccaccggcgcggtggagctcctgagcgaacagacggcatggccggaggccgggcggccccgccgggccggggtgtcgtcgttcggcatcagcgggacgaacgcgcacctgatccttgagcaggctccgctgccgacggcagcggagcggcccggtgacgccgagcccgttccggtcgagcctgccgcggtggtcccgtggatcgtctcggggcgcgaccggcatgccgtgcgcgcgcaggcggaacgactgcgcgcacacgtggtgagccaccctgaccggagggtggcggacatcggtttctcgctgctgaccagccgcgccgtgctggagcaccgagcggtggtactcggcggtgaccatgccgaactgctggccgggctgacggccctggcacgggacgaacccgcaccgggcgtggtggaggccctggacgcggccgagccggggcgcaaggtggtgttcgtcttccccggtcaggggtcgcagtgggccgggatggcgctggaactgatggagtcctcgcccgtgttcgcacggcggatgggcgagtgcgccgatgcgctggctccgctggtggagtggtcgctgccggacgtgctggcggatgagcgagcgctggcccgtgtcgatgtggtgcagccggtgctgtgggcggtgatggtgtcgctggccgagctgtggcgttcgtacggtgtggtgccgtcggcggtggtgggtcactcgcagggtgagatcgcggcggcgtgtgtcgcgggtggcctgtccctggcggacggggcaagggtggtcgtgctgcgcggcaaggcgctgctcgccttgtcgggccggggcggaatggtgtccgttccggtgcccgccgaccggctgcgggaccggcccggggtctccatcgcggcggtgaacggcccatcctcgacagtggtgtccggcggcgacgaggtgctggacgcggtgctggcggagttcccggccgccaagcgcatcccggtggactacgcctcccactcgccccagatcgacgacatccgggacgaactgctgaaggccctggcgccgatcgagccgcgcaccgcggcgatccccttccactccacggtgaccggacggcccatcgacaccgccgacctggacgcggactactggtatcgcaatctgcgcgagaccgtggagctcgagcgggtcatccgtacggcggtcgaggacggccaccacaccttcatcgagatcagcccccacccggtgctgaccacgggcctgcgcgaaacactcgacgacgcggacgcgcacggcggcctcgtactggcctcactgcgccgggacgacggtggccctacccgcttcctcaccgccttggccgaggcgtacgcacacggcgtcgaggtcgactggctgccgctgttcccgggcgcccgccgggtggatctgccgacgtacgccttccagcgcgagcgctactggctggacgcgcccaccgccgaggcccccaccagcgcgatcgacgcggaattctgggccgccgtcgagcgcgaggacctcgagtcgctcgccgcgacgctgcgcgtcgacgggcagccgctgcgcgaagtgctgcccgccctgtcccagtggcggcgcgaacgccgtgacgtctccaccatcgactcatggcgttacacgatccggtggaagccgctcaccccgcccgccacttcaccgaccggcacctggctggtcgtggtctgccatgccgaggccgggcacgagtgggtcgcgggggtgaccgacgcgctgacccgtcacggtgccgagccgctcgtggtcgttctcggcgagcccgaactggaccgtgccgcgctggccgcccggctgggcggcgtactggccgacacccccaggatcagcggtgtggtgtcgctgaccgcgctggacgagagcccgcacccggcgtacccctcggtcccccagggatacgcgatgacgctgctgctctcgcaggcgctcggggacgccagggtggaagctccgctgtggtgcctcacccagcgcggcgtctcgctcggcgatgccggaggcagtggcagtggcagtggcactggcgacggcaggggcaagggcaagggtgatgtggccgtcagccggaagcaggccctgacctggggtctcggcaaggtgatcgctctggaacagcccctgcgctggggcggtttgatcgacctgccggagggcgtggccccgcatacccaggactaccttgccggtgtgctgtccggcacctcggacgaggaccaggtggcgatccgcccgacggggctcttcggccgtaggctggcccacgcgccggcccgcgagcgcggcgggggctggcaaccccgcggcaccgtactggtcaccggtggcaccggagcgctgggcggccatgtcgcccggtggctggccggccagggggctgaacacgtggtgctgaccagtcgccggggcatggccgcgcccggcgccgagcggctggccggggagctggaggcgctcggcgcccgggtgacggtggcggcgtgcgacgtcggtgaccgggacgccctggccgggttgctggccgaggtcggcccgctgaccgctgtggtgcacaccgcggcggtgctcgacgacggcacgctgaactcgctcaccaccgaccagctgcaacgcgtgctgcgcgtcaagaccgacggcgcggtgcatctgcacgaactgacgcgggacatggagctgtccgcgttcgtgctcttctcctcgctgtccggcactctgggcgcacccggtcagggcaactacgcacccggccatgtcttcgtggacacgctggccgagcagcggcgggccgagggcctggtggccacctccatcgcctgggggctgtgggccggtgacggcatgggcgagggcggtgtgggcgacgtggcccgccgccatggcgtaccggagatggcgccggagatggcggtcgccgccatggcacgcgccgtcgagcaggacgacaccgtcgtcacggtggccgagatcgactgggaccggcactacgtcgcgttcaccgcgacccgccccagcccgctgctgtccgacctccccgaggtgcgtgcgctggtcgacgccggagtcggccaggagagcgccgagccgggccacgagcgctcggaattcgcggagcggctcgccgggatggccgagaccgaccggaaccacgcgttgctggacctggtccggcgccatgtcgccgtcgtactcggacacaccggtccggacgcgatcgaccccggccgggccttccacgagatcggcttcgactcggtcaccgcggtcgaactgcgcaaccggctcaaccgggccaccggcctacggctgcccgccaccgtgacgttcgaccagcccaccccgctggcgatggcgcagtacctccgcggcgaactgctgcacgacggccaaggccgatcggcccccgccctcccggtccgcgcgaccggcgcggtggacgacgagcctatcgcgatcgtggggatgagctgccgcttccccggggacgtcgcgtcccccgaggacctgtggcggctgctcgccgacggttccgacgccatcggcgagttccccgagaaccggggctgggacaccgcgcacctcttccacccggaccccgaccaccgaggcacctcctccacccgagcggccgcgttcgtctccggggccggtgagttcgacgccggattcttcgggatctccccgcgggaagcggtggcgatggacccgcaacagcggctgctgctcgaagtgtcatgggaggcgctggagcgggccgggatcgaccccacgaccctgcggggcagcgagaccggcgtgttcacggggacgaacggtcaggactacgcgtcgttgctgaaggcggacgagacgggtgacttcgagggccgggtgggcaccggcaactcggcatcggtcatgtccggccggatctcctacgtcctcggtctcgaaggccccgcgctgaccgtggatacggcgtgctcgtcgtcgctggtggcattgcacctggcggtgcgggccctgcggtcgggcgagtgctcactggccctggcgggaggcgcgagtgtcatgacgaccgccggcatcttcgtggagttctcccgtcagcgcgcgttggcggccgatggacgctgcaaggcgttcgcggcggcggcggacggtaccggctggggtgagggtgccggaatgctggtggtggagcggttgtcggatgctgagcggcttgggcatcgggtgttggcggtggtgcgtggttctgcggtgaatcaggatggtgcgtcgaatggtttgacggcgccgaatggtccgtcgcagcagcgggtgatccggcaggcgctggcgagcgcggggctgtccacggtggatgtggacgcggtggaggcgcacggtacgggtacgacgctgggtgatccgatcgaggcgcaggcgttgctggccacgtacggtcagggccgggattcggaccggccgttgctgctggggtcgatcaagtcgaacatcggtcacactcaggcggccgccggtgtggctggtgtgatcaagatggtgatggcgatgcgccacggtgtgctgccgcagagcctgcacatcgatgagcccactccccacgtcgactggtccaccggcgcggtggagctcctgagcgaacagacggcatggccggagaacacacggccccgccgcgccggggtgtccgccttcggagtgagcggcaccaacgcgcatgtgattctggagcaggcccccgagccgaccgccgcccagcccgaactctcgccggaacgcgacgaaatgagggccgtgccgtgggtggtgacgggtgcgagcgaggccggagtccgcgcacaggccgcgcgcctcatggcctttgtcgacgaccggccggaactccgcccggtgaacatcggctggtcgctggcctcgacccgcgcggccctgtcacaccgtgccgtggtcgtaggtgctgaacgtacggaactgctgcgtgagctggaggccgtggccagtggcagcgtcacggtcggcgaggcccgcacgcattccggggtggtgttcgtcttcccggggcaggggtcgcagtgggttgggatggcgttggagttggtggagtcgtcgccggtgttcgcggggcggatgcgtgattgtgcggatgcgttggccccgtttgtggagtggtcgttgttcgatgtgttgggtgatgaggtggcgcttgggcgggttgatgtggtgcagccggtgttgtgggcggtgatggtgtcgttggcggagttgtggcgttcgtttggtgtggtgccgtcggtggtggtggggcattcgcagggtgagatcgcggcggcgtgtgtggccgggggtctgtcgttggaggacggtgcccgtgtggtggccttgcggagcagggcgttgctggctctgtcgggtcggggcggcatggtgtcggttccggtttctgctgaccggctgcggggtcgtgtggggttgtcggtggcggcggtgaatggtccggtgtcgacggtggtgtcgggggctgttgaggtgctggatggggtgctggcggagttcccggaggcgaggcggattccggtggattatgcgtcgcattcggtgcaggtggaggggatccgggagggtttggcggaggcgttggcgccggttcggccgcgtacgggtgaggtgccgttctattcgacggtgaccggccggctgatggacaccatcgagttggacgcggagtactggtaccggaacctgcgcgagacggtggagttccagagcgcgatcgaggggctgctggagcttggccatacggtgttcgtcgaggccagcccgcatccggtgctgaccattggcatccaggacaccgccgacaccaccgacaccgacatcgtcgtaagcgggtcactgcgccgcgacgacggcggtcctgtccgcttcctcagcaccgtcgggcgactgttcaccgagggcgtgccggtggagtggcagccgctgttcgccgcggccggggcgcgaaaggtcgatctcccgacctatgcgttccagcatgagtggttctggctggatccggtgcgcggcgcgagtgatgtgggcggcgcgggccttgccggtctcgctcaccccttggtgagcgcggtgttgccgctgcccgaatccgatggctgtgtgctgaccggctcgctctcctcggccacccatccttggctgcgtgaccacgccgtgctggacaaggtgttgctgccgggcaccgggttcgtggaactggcccttcaggccgggctgcacctgggctgccggacgctggatgagctgaccctgcaggcgccgctcatgctgcccgcgcacggagacgtacagatccaggtggcggtcggcggaccggacgacagcggccgccggccggtcacggtgtactccaggccgggcaaggaccggacctggatgcggcacgccaccggcagcatcagccccgtcggtgaaacggccaccgtggaccgggcggtgtggcccccggtcggcgccacaccggtcgagctcaccgatgtctacgccgagatgagcacgcacggttacgcgtatgggcccgtcttccaggggctgcgcgccgcatggcgacgtggcgacgaggtgttcgccgaggtggtcctgcccgagacggccgagagcgacgcgggtcgttgcgccatccaccccgccctcctcgacgccgccctgcacggtgccggactgggcacgttcgtgaccgaaccaggccgaccgcaccttccgttcacctggaccggtgtcaccctgcacgccgtcggtgccaccaccttgcgggtcgtcctgtcgcccgccgggccggacgccatctcgctcctggccatggacggcacgggagcgccggtgctgacggcggactctctggccctgcgcccggtgtccgagggcgggctcggcggctcccacgacgactcgctgttccgcgtggactggaccgagctcaccctggacgcctcggacgcctcggacgcaccggaggtgtcggatgaagcggccttcccggtcgtcgagtccgtggcccagctggccggggtggcggcggcccggagcgggcgcggggccgtggtgttcaggctttccaccacggagaccacaggaggcgccgccgaggagagcccggaggacgtctacgcgctcaccagccgtgtcctcaaggtcgcgcaggcgtggttggcggacgaccggttcggggacgcccgcctcgtcgtggtgacgcggggcgcggtcgcgaccacgcccggagagaacccggagagccttgccgccgccgcggtctggggcctcatccgcaccgcgcagaccgagaaccccggccgtttcgtcctcgtggacacggtggacgaggatccgtcggcgttgccgggggtgctcgccaccgatgagccacaggtggcgatccgggcggggaaggcgctggtgcccaggctggtacgggccacctcgtcggcgttgccggtaccagctgagacggacacctggcggctggagaccgacggtcagggcactctggagaacctggtcctctcgccccgcgccgaggcgtccaggccacttgccgcacatgagatccgggtggccgtgcacgcggccggggtcaacttccgcgatgtactgctcgctttggggatgtacccggacaaggccggtctgctgggcagcgaagccgccgggacggtgctggagatcggctccggagtagtgggagtggcaccgggagaccgggtgatgggtctgttctccggtgccttcgcgccggtggcgatcaccgatcaccgactggtggcaccgatcccggaggggtggtccttcccgcaggccgccgccaccccgatcgccttcctcacggcgatgtacgccttgatcgacctggccgaagtgcggagcggcgagtcggtgctggtgcacgcggcggccggtggcgtcgggatggcggcagtgcaggtggcgcgctggctgggcgccgaggtgttcgccaccgcgagcccggccaagtgggatgcggtgcgcgcatgcggggtcgccccgcggcggatcgcttcctcccgctcgccggagttcgcggaccgcttccgctcggacgcaccggacggtgtggatgtcgtactcaactcgctgaccggtgaactcctcaacgcgtcgctcggactgctgcgtcccggtggacggctgatcgagatgggcaggaccgaactccgggacgcacaggaggtgatggcgcgccacggtgtgtcgtaccgggccttcgaactgctcgacgccggtcccgaccgtatcggccgactgctcaccgagctgctcgccctgttccaccagggcgtgttcaccccgctgccactgcgcgtccaggacgtacggcaggcgagtgacgctttccgccacctctcccaggcgcgccacatcggcaagctggccctcaccatcccgcgaccgttgtccggcggcaccgcactgatcaccgggggcaccgggacactgggcggtctggtggctcgccaactggtgcgggagcacggcgtgacggagctggtgctggccagccgtcgtggtgacaccgctccgcaggcggcggagctgctcaccgagctggaggccgccggggcgcgggtgcgggtggccgcatgcgatgtgtcggaccgggacgccatcgccgcactcgtcgcctcgctgccgaacctgcggagcgtggtgcacacggccggtgtcctcgacgacgccgtgatcgggtcgctcaccccggagcggctgcggacggtactgcgtcccaaggcggacgccgcatggcatctgcatgaactgacccgggaccgggaccttgccgagttcgtgttgttctcctcggcggccggagtactcggtggcccagggcagggcaattacgcggcggccaacgccttcctggacgcgctggccgcgcgccgccgggcacagggactgcccgcgacctcgctggcctggggcttctgggagcagcgcagcggactgaccgaacacctgaccaccgatcggctcgcccgggccggcgtcctgccgctgtccaccgacgaggggctggtcctcttcgacgacgcccgcgcgaccggcgacaccctgctggtgccgatgcgttacgaaccgtcctcgccgggccctgagccggtacccgccctgctgcgtggcctcgtacgcgctccgctcgcccgcgcccttccgggcccggccgatggtgtgggcagcggtgtggcggagggcctcacagggctggcggcggacgaacgcctcggcgcactgctcgacctggtccgccgggaggcggcggccgtgctcggccacggcggtccggaatcggtgacaccccagcgtccgttcaaggaactcggcttcgactcgctctccgccgtggaactgcgcaaccggctgcgcgcggcgaccggccgacggctggaggccacccttgtcttcgaccaccccactccggccgtgctcgcacgccacctcgacgccgagctgttcggcgccaccgacgtggcggcgcccgtaccagcaccggcggtcgcgcacccggccgacgagccgatcgccatcgtcggcatgagctgtcggctcccggccggggtggactccccggaggcgctgtggaagctgctggtgagcggcacggacgcgatatcggagctgccccccgaccgcggctgggaccttgacaggctctacgaccaggatccgagccggcccggtacgacatacgccaagaccggtggcttcctgaagaacgcggcggacttcgacgcgggattcttcacgatctccccccgagaggcgctggccgcggatccccagcaacggctgtggctcgaggcgtgctgggaagccttcgaacgcgccggtatcgatccgctcgccctgaagggcacccgaaccggggtgttcgcgggtgccgtttcgacgacgtacggcgcgggtcaggccgccactccggacggctccgaggggtacctgctcaccggcaactccacctccgtgatctccggccgcgtggcctacaccctcggcctcgaaggccccgccgtcaccgtggacaccgcgtgctcgtcctcgctggtcagcgtgcactgggcgtgtgagtccctgcgccggggcgaaagcacactggcgctggcgggcggtgtggcggtgatgacgacaccggacctgctggtcgaattctcccgccagcgcggactcgcaccggacgggcggtgcaagtcgttcgccgccgccgctgacggcacagggttcgccgaaggcgtcggggtgctcgtcctggaacggctgtccgacgccacgcggaacggccaccaggtgctggcggtgatccgcggctccgccgtcaaccaggacggcgcgtccaacggtctgaccgcgccgaacggcccctcgcagcagcgggtgatccggcaggcgctggtgaacgccggactcgcctcccaggatgtcgacgtggtggaggcgcacggtacgggtacgacgctgggcgaccccatcgaggcgcaggctctgctggccacctacggccaggaccgggatccggatcggccgctgctgctgggctccgtgaagtccaacatcgggcacacccaggcggccgcaggtgccgccggactcatcaagatggttctggcgctgcgcaacggcgtactgccgcgcaccctgcacgtcgacgagccctccccgcacgtcgactggtccgccggggccatggagctgctgaccgagcagaccgcgtggcccgaccgggaccacctgcgccgggccggggtgtccgcgttcggagtgagcggcaccaacgcccatgtgatcctcgaacaggccccggagccggatgagaacggcgaaccggacaccgtccggtcgtggttgcccgcggtgccctgggtgctgtcgggcgcgggagcggccgggcttcgggcccaggcccagcggttggcgtccttcgtgcgggagaaccccgggctcgaccccgtggacgtgggctggtccctggtcgcgacccgcgccgccctgtcgcaccgagccgtcgtcgtgggcgcggaccgcacggaactgctgcgcgagctggccgcggtggaatccgtgggcgccgccgaggcggagcgcgacgtggtgttcgtcttcccggggcaggggtcgcagtgggttgggatggcgttggagttggtggagtcgtcgccggtgttcgcggggcggatgcgtgaatgtgccgatgcgctcgccccgtttgtggagtggtcgttgttcggtgtgttgggtgatgaggtggcgctcggtcgggttgatgtggtgcagccggtgttgtgggcggtgatggtgtcgctggcggagttgtggcgttcgtttggtgtggtgccgtcggtggtggtggggcattcgcagggtgagattgcggcggcgtgtgtggcgggtgcgttgactttggaggatggggcgcgtgtggtggccttgcggagcagggcgttgctggctctgtcgggtcggggcggcatggtgtcggttccggtgtccgctgatcggctgcggggtcgtgtggggttgtcggtggcggcggtgaatggtccggtgtcgacggtggtgtcgggggctgttgaggtgctggatggggtgctggcggagttcccggaggcgaggcggattccggtggattatgcgtcgcattcggtgcaggtggaggggatccgggagggtttggcggaggcgttggcgccggttcggccgcgtacgggtgaggtgccgttctattcgacggtgaccggtcggttgatggacaccgtggggctggacggggagtactggtatcggaatctgcgtgagacggtggagttccagagcaccgtcgaagctctgatcggccagggccacacggtgttcgtcgaggccagcccgcatccggtgctgaccgtcggcgtccaggacaccgccgacgcgatggagacccccatagtggccaccggttcgcttcgccgggacgagggaggcgtacgacggttcctgacgtcactggctgaggtatccgtccatggcatcgaggtcaactggcagacggtcttcgacggcaccggcgctcggcgagtcgacttgcccacctacgcgttccagcgtgagcggttctggctggtgccatcgacgggcacgggcgacgcgtccgggctgggcctgggcgccgttgaccatccgctgctgggcgcggcggtgccgcttccggacgcggacggctgtgtgctgaccggtgcgctgtcgctggccgggcagccatggctggccgaccactccgtcctcggcatggtccttctgccgggcaccgcgttcgtggagctcgcgttgcaggcgggggcgcggttcggctgcggcactctggaagagctgacgttgcatgagccgctcgtcctgcccgagcgggagaccgtgcagctccaggtgtcggtcggaggctcggacgacttcggaggccgccccttcacggtgttctcccgctgtgagggtgagtggatacgccacgccgggggcaccctgcgtgtgggcgagcgtggcgatccgcccgcgaacccgtcggtctggccaccggccgatgcccggccggtcgatgtcgccgagttgcacacgacgatggccgagcggggctatcagtacgggcccgccttccagggcctgcggaaggcatggatccgtgacagcgaagtgtttctcgacgtcgcgttgcccgagcaggtgaggggcgacgcggcccgctgcggagtgcatcccgcgttgctggacgcggccctgcaaggcatcggcctcggcgccttcgtcaacgaaccgggccaggcccatctccccttctcctggagcggggtgaccctgcacgcggtgggcgccactgccgtgcgggtgacactcagcccggccggaccggacacggtggccatccggatggcggacaccatcggggcgcccgtgctgtccatcgacgcgctggcgatgcgtccgctcgcggagcagcggctgctcgaggcgggtggcagccgcggcgatgcgctgttccggctggagtggaaggagcttcccgtccccacgggggccaccggcccacgggcgcagtcctggggcctgctgggcggccacgacgagcctcgactgaccgcggcgctgaccgcggccggtgtgtcgccacaacgccatcgggacctcgcctccatcgaccaggtgccggatgtgctggtcctgtcgtgtccgcccgaggcggatggcggcccggccccggaagccacctcgtccgccctccgccgagtgctggaagtggtgcgggagtggctcggggacgcgcggtacaccgatgcccgactgatggtgctcacccgccgcgcggtggccacatccaccggtgacgacgtggaggatctggcggcggccgctgtacggggactcctgcgcaccgcacaacaggagaaccccgaccggctcgtcgtgatcgaccatgacgactcggaccttgaggtgctccccgtggtgctcgggacaggggagcccgaagcggccatccgggccggtaaggtgctggtgcccaggctggtcaaggcggccgtatcggaagggaaggcccctgcctgggacgccggcaccgtgctgatcaccggcgggacggggacactcggcggcctggtcgcccgccatctggtgaccacccatggcgcgcgtgacctggtgctggccagtcgcggaggtgacaccgcgcccggcgccgtggaactggccaccgaactggaggcgctcggtgcccgcatccgcgtcgccgcctgcgatgtggccgatcgtgcccagctgaccgcgctgctcgacaccattccggcgctgcgtgctgtcgtccacaccgcaggtgtggtggacgacggtgtcatcggctcgatgaccgccgaacgcgtggagaccgtcctacggccgaaggcgaacgcggcgtggcacctgcacgcgctgacccgccacctggacctggacgcgttcgtactgttctcctccgccaccggagtgctgggcagcgcgggacagggcaactacgccgcggccaacgccttcctcgacgcgctggccgtgcaccggcgcgcccaggggctccccgcggtgtcggtggcatggggcctgtgggagcggcgcagtggcctgaccgcacatctgtcggagcaggacgtggcccgtatgaccagcacgggcgccgttcccctctccgacgaacgcggtctcgagctgttcgacgccgcgtgccggagtggcgaacccacactcgtggccaccccgttgcaccttcgtgcggtggcggccaccggtacggtgccccacgtgctcagcgcgctggcaccgaccccgccacgccgggccgccgaggccggtgacggtggagtggctctacggcagagccttgccgagatgtcgggcgcggaacagagccagaccgtcctggggctggtacgcgggcaggtcgccgccgtgctgcggcacccggacccgtcggcgatcgacacggcgcggacgttccaggagatcggcttcgactcgctgaccgcggtggagctgcgcaaccggctgggcgccaccaccgggatcaggctggccgcgaccgcgatcttcgactatccgacacctgccacgctggcacagcacctgctcgccgagatcgtgccggagaccgccgacccggtcgcggcccggctcggcgagctggacaaggtggccgccatgatttcggcgatggccgaggacgacaccctgcgcgagcagttgtcctcgcggatggagaccatcgtcgcgatgtgggccgacctgcaccgtccggagcggccgggcacggttgagcgggacctcgaatccgcctcgctcgacgacatgttcggaatcatcgaccaggaactcgatgggtcatgagcagcgagaacgtccgaccggaaatcgaggggactggcacgcggatgtcgaacgacgaaaaggtactcgagtacctcaagaagctcaccgccgatctgcgccagacgcgtcagcgtctccaggacgtcgaggccaagagccgcgagccgatcgcgatcgtcggtatgagctgccgtttcccgggtggggtgagctccccggaagacctgtggcggctgacggagtctgcggtggacgcggtctccggtttccccacggaccgaggctgggacctggacggtctgtacgaccccgacccggatcgcgcgggccggtcgtacgcccgagagggcgcgttcatccccgatgcaggccacttcgaccccggcctcttcgggatctcgccacgtgaggcgctggcgatggatccgcagcagcggctgctgctggaggcatcgtgggaggccctggagcgggcgggtattcccaccgattccctgaagggcagccggaccggggtgttcgccggactgatgtcttccgactatgtctcgcggctgtccgcggtcccggacgaactcgaggggtacgtcggaatcggaagcgcggcgagcgtcgcctccggccgcgtgtcgtacaccctggggcttgagggcccggcggtcaccgtggacacggcgtgttcgtcgtcgttggtggcgttgcatctggcggtgcaggcattgcggtcgggtgagtgctcgctggcgctcgcgggcggtgtcacggtgatggcgacacccggcaccttcgtccagttctcccgccagcgcggcctggccgccgacggccggtgcaaggcgttcgcggcgggggccgacggtaccggctggggcgaaggcgtcggcatgctggtggtggagcggttgtcggatgctgagcggcttgggcatcgggtgttggcggtggtgcgtggttctgcggtgaatcaggatggtgcgtcgaatggtttgacggcgccgaatggtccgtcgcagcagcgggtgatccgtcaggcgttggcgaatgcccgtttgtcggcggtggatgtggatgcggtggaggcgcatggtacgggtacggcgttgggtgatccgattgaggcgcaggcgttgttggccacgtatggtcagggtcgggatgtgggtcggccgttgtggttggggtcggtgaagtcgaacatcggtcatacgcaggcggctgcgggtgtggctggtgtgatcaagatggtgatggcgatgcggcatggggtgttgccgcggacgttgcatgtggatgagccgtcgccgcatgtggattggtctgctggtgcggttgagttgttgacggggcaggtggcgtggccggaggtggatcggccgcgtcgggcgggtgtgtcggcgttcggggtgagtgggacgaatgcgcatgtgattgtggagcaggcgcctgaagtggcggagtctgaggctgaaggtgtggtgttgcctgctgtgccgtgggtggtgtcgggtgtgggtgaggtggcggtgcgggcgcaggtggagcggttgcgggcctttgcggaccggaatccgggtctggatccggtggatgtggggtggtctttggcgactggtcgtgcggggttgtcgcatcgtgcggtggtggtgggtgcgggtcgtggtgagttgttgggggctttggagggtgtgccggtggtgggtgttccggtggtgggtgggttgggtgtgttgtttgcgggtcaggggtcgcagcggttggggatggggcgtgggttgtatgaggggtatccggtgttcgctgcggtgtgggatgaggtgtgcgcgcagctggatcggtatttggataggccggtgggtgaggtggtgtggggtgatgatgccgggttggtcggggagacggtgtatgcgcaggcggggttgttcgcgcttgaggtggcgttgtatcggctgatcgcttcgtggggtgtgagggcggattatctgctgggtcattcgattggtgagttggctgcggcgtatgtggcgggtgtgtggtcgttggaggatgcggtgagggtggtggtggcgcgggggcgtttgatgcaggcgttgccgtcgggtggtgcgatggttgcggtgggggcgtcggagggtgtggtgcggccgctgctgggcgagggtgtggtggttgcggcggtgaatggtcccgagtcggtggtgctgtcgggtgatgaggatgcggttcaggttgtggtggatgtgttggctgggcgtggggtgcggacgcggcggttgcgggtgagtcatgcgtttcattcggctcgtatggacggcatgctggcggagttcggtgaggtgcttcggggcgtggagttccgtgccccgagcgtgcccgtggtgtcgaacgtgtccggtgtggtggcgggcgaggagttgtgttcgccggagtattgggtgcggcatgtgcgggagacggtccggttcgccgatgggctggagacgctgcgcgagctgggtgtgggttcgttcctggagttgggaccggacgggacattgaccgcgctggccgacggcgatggtgtgtcggcgctgcgccgggaccgtccggaaccgactgcggtaatggctgctttgggtgggttgtatgtccggggtgtggaggtcgactgggacgcggtgttcccgggtgctcggcgggtcgatttgccgacgtatgccttccagcgtgagcggttctggctggaaccggccgctgagcagcctgcgacgagcgcggtggacgcggcgttctgggacgcggtcgagcggggcgatgcggagattctcggggttgacgttgagcagccgttgagtgccgcgttgcccgcattggcgtcgtggcgacgggcgcggcaggaagagtcggtcatcgacgcatggcggtatcggctgacctggaccccggtcgcgggtctctcttcgcagctctccggcgtgtggttggtggtggtcgagccggacgaggcggagccggacgtcgtcgccgcgctgcggggcgccggcgccgaggtgcgtgtcgtaacgatcgatgagctggacgcgggcccggtcgcgggcgtggtgtctttgttgtcggtcgagacgacggtgtcattgctccaggcccttgtggcagaggggggcgatgcgccgttgtggtgtgtcactcggggtgcggtctccgtggtggacggggatgtggtggatccgcatgcgtcggccgtctggggtttgggccgtgtgatcggtctggagcatccggaccgttggggcgggctgatcgatctgcccaccgcatggggtgagcgaacctccggcatgttgtgctcggtgctttcgggcgccacgggtgaggaccacacagcgatccgtggcgacgaggtgttgggttgtcgtctgagccgtgcgacgacgtcggcaccggggccgtccactgcctgggaagcgtcggggaccgcgctgatcaccggtggaacgggtgccttggggagccatgtcgcccgatggctcgcggataccggcgtcgaagagatcgtgctgacgagccgacgaggcgcggacgctcccggagcacgggaactggtcgccgaactgtcggccatgggcgtatcggcccgcgtcgtggcgtgtgatgtggccgatcgggacgcggttgcggagctgatcgagaccattccggacctccgcgtggtcgtccacgccgcgggagtaccgagctggggtgcgttgagcacacttaccgcacagggccttcaggatgggatgcgggcgaaggtcgcgggagccatccacctggatgagctgacgcgcgatatgcgcttggacgcctttgtgttgttctcgtcggtggcgggggtgtgggggagcggtagtcagtcggcgtatgcggcggcgaacgcgtttctggatgggttggcgtggcggcgtcgtggtgttgggttggtggcgacgtcggtggcgtgggggatgtggggtggcggtggtatggcggttgggggtgaggagtttctggttgagcgtggtgtgtcggggatggctccggggtcggcggtggctgcgttgcggcgggcgctgtgtgatggtgagacggcgcttgtggtggcggatgtggattgggagcggttcgggccgaggttcaccgcgttgcgtccgagcccactgctgagcgagctgatccccgacaccgtcggctcgggggttccgctgggtgaattcgcggcccgtttccagaccatgtccgagggcgagcgcatgcgcgcggccgtcgagctggtgcgtgtttcggccgcggccgtgctggggcaccagggcccggaggccatcgatcccgtcaggacgttccaggagatcggcttcgactcgctgaccgcggtggaactgcgcaaccggatcgccacggctaccggtatccgcccgccggccacgatggtcttcgactatccgactcctgtggccctcgccgaatatctgagcgtggaattgctcggttcgccgcaggacagtgtgccgccgttgcaggtggccgcgccggacgacggtgatcccattgtcatcgtcggcatgagctgccgcttccccggggacgtcgagtctcccgaggatctgtggcggttgatcgactccgacggcgatgccataacggcctttccgacggaccgtggatgggacctgaccggcctcttcgacacggctgtgggggagtcggggacgtcgtatgcgcgtgttggtggcttcgtccacgacgcgggtgagttcgatccggccttcttcggtatctcgccgcgtgaggcgaccgcgatggatccgcagcagcggctgctgctgcacgcggcatgggaggcgttcgagcgggccggtatcccggccgcctcggtcaggggcagcaggactggagtgttcgtcggagcctcgccgcagggctatggcgccgccgaagcgtcggaaggctatttcctcaccggtagttcgggcagtgtcatttcgggtcgcgtgtcgtacacgctgggtcttgagggcccggcggtcacggtggatacggcgtgttcgtcgtcgttggtggcgttgcatctggcggtgcaggcgttgcggtcgggcgagtgttcgctggcgctcgcgggcggtgtcacggtgatggcgacacccactgctttcgtggagttctcgcgtcagcgtgggctggccgccgatggccgctgcaagtcctttgccgctggtgcggatgggacaggttggtcggagggtgttgggctgctgctggtggagcggttgtcggatgcggagcggcttgggcatcgggtgctggcggtggtgcgtggttctgcggtgaatcaggatggtgcgtcgaatggtttgacggcgccgaatggtccgtcgcagcagcgggtgatccgtcaggcgttggcgaatgcccgtttgtcggcggtggatgtggatgcggtggaggcgcatggtacgggtacggcgttgggtgatccgatcgaggcgcaggccctgttggccacgtatggtcagggtcgggatgtgggtcggccgttgtggttggggtcggtgaagtcgaatattggtcatacgcaggcggctgcgggtgtggctggtgtgatcaagatggtgatggcgctgcggcatggggtgctgccgcgaacgttgcacgtcgatgaaccctccccgcatgtggactggtcgtccggggcggtcgagttgttgagcgagagggctgcttggccggagatgggccgaccgcgtcgggcgggcgtgtcgtcgttcggggtgagcgggacgaacgcgcatgtggtgttggagcaggctcctggggcggtggaggagtctcggggcgagggtgttgcgttgcctgctgtgccgtgggtggtgtcgggtgcgggtgaggtggcggtgcgggcgcaggtggagcggttgcgggccttcgcggaccggaatccgggtctggatccggtggatgtggggtggtctttggtggccactcgttctgggttgtcgcatcgtgcggtggtggtgggtgcggatcgtgaggagttgctgggtgggttgggttcggtggtggtgggtgttccggttgcgggtgggttgggtgtgttgtttgcgggtcaggggtcgcagcggttggggatgggtcgtgggttgtatgaggggtatccggtgttcgctgcggtgtgggatgaggtgtgcggggagctggatcggtatctggataggccggtgggtgaggtggtgtggggtgatgatgccgggttggtcggggagacggtgtatgcgcaggcggggttgttcgcgctggaggtgtcgctgtatcggctgatcgcttcgtggggtgtgaggggggattatctgctgggtcattcgattggtgagttggctgcggcgtatgtggcgggtgtgtggtcgttggaggatgcggggagggtggtggtggcgcgggggcgtttgatgcaggcgttgccgtcgggtggtgcgatggttgcggtggcggcgtcggagggtgaggtgcggccgctgctgggcgagggtgtggtggttgcggcggtgaatggtcccgagtcggtggtggtctcgggggatgaggatgcggttgaggcggttgtggatgtgttggctgggcgtggggtgcggacgcggcggttgcgggtgagtcatgcgtttcattcggctcgtatggacgggatgctcgcggagttcggtgaggtgcttcggggcgtggagttccgtgccccgagcgtgcccgtggtgtcgaacgtgtccggtgcggtggccggtgaagagctctgctcgccggagtattgggtgcgtcatgtgcgggagacggtccgattcgcggatgggctggagacgctccgtgagctgggtgttggttcgttcctggagttggggcctgacgggacgttgaccgccttggcggatggcgatggtgtgcctgtcttgcgtcgggatcgtccggagcctctgaccgttatggcggctttgggtgggctgtacgtccggggtgtccagatcgactgggatgcggtgttcccgggtgctcggcgggttgatttgccgacgtatgccttccagcgtgagcggttctggttggagccgtcccctgagcagcccacgacgagcgcggcggacgcggcgttctgggatgcggttgagcgtggggatctcggttctttcggtatcgatgccgaacagccgctcagcgccgcactgcccgccctctcgtcctggcgccgccgtcaccaggagaggtcactcgtcgagtcctggcggtaccgcctcgactggtccccgatcggcaccgcttccgagcagccgagtctgcgcggcacgtggctggtggtgggcgagggcggagacgacgtggtcgccgtgctgcgggctgcgggggccgatgcgcgagttgtgacaatggcggagctgggcgaggtcgcggctgcgggtgtggtgtcgttgttgccggtcgaggcgacggtgtcactggtgcaggcactggggacggccggggccgatgcgccgttgtggtgtgtgactcggggtgcggtgtcggtggtcgatggtgatgtggtggatccggggcagtcgggggtgtggggtcttggccgggtgatccgtttggagcatccggatcgttggggtggtctgatcgatgtgccggtggtggtggatgaggaggccggggcttggttgtgccgggtgttgggtgggggtacgggggaggaccaggttgcggttcgtggtggtggggcgtggggtgctcggctggtgcgggtgtcgggctcgggttcgggatcgggtggggcggttgtgtggcggggtcgaggggcggcgttggtgacgggcggtacgggtgcgttgggtggtcatgtggcgcggtggttggccggtgctggtgtggagactgttgtgctggcgagtcgtcgggggatggctgcgccggatgcggagcagctggtcgcggagttggaggggttgggtgttgcggtgcgggtggtggcgtgtgatgtggcggatcggggtgcggtggcggagttgttggaggggattggggatttgcgtgtggtggtgcatgcggcgggtgtgctggatgacggtgtgttggagtcgctgacgtctgagcgggttcgtgaggtgatgcgggtcaaggcggagggtgcgcggtatctggatgagttgacgcggggttgggatctggatgcgtttgtgttgttttcttcggctgcggggactgtgggtaatgcgggtcaggggagttatgcggcggcgaatgcggtgttggacgggttggcttggcggcgtcgggcggaggggttggtggccacgtcggtggcttggggagcctgggccgacagcggcatgggggctgggcacgcacgggccatggcaccacggctggcgttggcagcccttcagcgagcgttggacgacgacgagaccgcactgatgatcgcggacgtggattggtcgagcttcggctcccggttcaccgccgtgcggcccagcccgctgctcggtgaattgctgggtggcgccgctcatcccgcgcccgcggtgggcgggttcgtcgaccggctacgggacctccccccggccgagcgggaacggacggtccttgagctcgtacgtggccaggtggccgtcgttctgggacatgccaccccgggggcgatcgacaccgcagcgacattccagtcagccggtttcgactccctgaccgcgatcgaactccgcaatcggctcatggcggccaccggagtgcagacacctgcctcggtcgtcttcgactaccccactccggaacttctcgccggccacctgcgggagcaactgctcggggcagggtcggcagcactctcgacgacggtcgccacggctccggtcgatgacgacccgattgcgatcatcggcatgagctgccgattccctggtggtgtcgactcgcccgaagagctgtggcggctcctggagtcggggacggatgccatttccgcctttccacaagaccgcggctgggacctcgtgggcggagtcgatggcgcgtcggtccgggcgggtggcttcctctacacggcggccgagttcgaccccgcgttcttcgggatctcgccgcgcgaggcgatcgcgatggatccgcagcagcggctgctgctcgaggcctcgtgggaggtcttcgagcgggccgggatcgccgcggacgcgttgcgggacagccccaccggagtgttcgtcggcaccaacggccaggattacgccgccctcgtcggtaacgcgccacagcgtgcggacggccatctggccaccggcagcgcggcgagcgtggcatccggccgactgtcctacaccttcgggctcgagggcccggccatcaccgtggacaccgcgtgttcgtcgtcgctggtggccatgcacctggccgcgcaggcgctgcgctcgggcgaatgccgtatggcccttgcgggcggcgccacggtaatggccacgcccaccgcgttcgccgagttctcccggcaaggcgcgttggccgctgatggccggtgcaaggcgttcgcggcgggcgcggacggcaccggctggggcgaaggcgtaggcattctgctgttggagcggctgtccgacgccgagcggaacggccaccgggtgctggcggtgatgcgtggctccgccgtcaaccaggatggtgcgtcgaatggtttgacggcgccgaatggtccgtcgcagcagcgggtgatccggcaggcgctggcgaacgcacgtctgtccacagtagacgtggacgcggtggaggcgcacggtacgggtacgacgctgggcgaccccatcgaggcgcaggctctgctggccacctacggccaggaccgggatccggatcggccgctgctgctgggctccgtgaagtccaacatcggccatacgcaggccgcggccggtgtggctggtgtgatcaagatggtgatggcgatgcgccacggcgtgctgccgcggagcctacacatcgacgagcccactccccacgtggactggacggccggacggatcgcactgctcaccgaaccgtccccctggcctctgacgggagcgccgcgacgcgccgccgtctcctcgttcggtgtgagtggcaccaacgcgcatgtgatcctcgaacaggcatctgcggtggccgaacccgaggaaaccgacacggcgcgaacacccgaaccgccagctgttccgtgggtgctctcggcacggagcgaggcggggctacgggcgcatgccctcaggcttcggtccttcgtgaacgccgatgctgatctgcgtccagtcgatgtcggctggtcgctggcgtcggctcgctcggtgttgtcacaccgtgcggtggtcgtgggcgcggaccgcgatgaactcctccgtgaactggaggccgtggccagtggcagcgtcacggtcggcgaggcccgcacgcattccggggtggtgtttgtcttcccggggcaggggtcgcagtgggttgggatggcgttggagctcctggagcattcgccggtgttcgcggggcggatgcgtgattgtgcggatgcgttggcgccgtttgtggagtggtcgttgttcgatgtgttgggtgatgaggtggcgctcggtcgggttgatgtggtgcagccggtgttgtgggcggtgatggtgtcgctggccgagttgtggcgttcgtttggtgtggtgccgtcggcggtggtggggcattcgcagggtgagatcgcggcggcgtgtgtggccgggggtctgtcgttggaggacggtgcccgtgtggtggccttgcggagcagggcgttgctggctctgtcgggtaggggcggcatggtgtcggttccggtttctgctgaccggctgcggggtcgtgtggggttgtcggttgcggcggtgaatggtccggtgtcgacggtggtgtcgggggcggttgaggtgctggagggggtgctggcggagttcccggaggccaagcggattccggtggattatgcgtcgcattcggtgcaggtggaggggatccgggagggtttggcggaggcgttggcgccggttcggccgcgtacgggtgaggtgccgttctattcgacggtgaccggccggctgatggacaccatcgagttggacggggagtactggtaccggaatctgcgtgagacggtggagttccagagcaccgtcgaagctctgatcggccagggtcatacggtgttcgtcgaggccagcccgcatccggtgctgaccgtcggcgtccaggacaccgccgacaccaccgacaccgccaccgacatcgtcgtcaccggatcgctgcgccgcgacgacggcggtccggcgcgcttcctcaccgcgctggccgagctgtccgtacgaggggtggcgacggactggcggcaggcgttcgaagggaccggcgcccgacatgtcgacttgccgacctaccccttccagcggcagcgcttttggatcgaacccactgccccggacgtggcccgggaggacgctcgcgtcaccactgcggacggcgagttctgggcggccgtcgagcgcgaagacgccgcatccctggcaacagccctggaggtcgacgacgcctcactgggcaacctgctgcccgccttgtcggcctggcgccgccggcggcacgagtggtccgcattggaggccgtccggtaccaggtcaactggaagcggctcgtcgatgaccgacccgcgatgttgtcaggtgcctggctggtcgtggtttcccaggccgacgccgaccatgagtgggtctccggcgtaagcgagacgctcgccgagtacggggccgagccagtggtgtgcccggtggacgagcgacacctggatcgtgccgtgctggccgaccggctggcgagcatgaccggtacgagcagcacgacgagcacggcgagtatcagcggcgtggtgtcgctggtcgccctggaccagcgcccgcacccggacttcgcctccgtgcccattggtttcgcgatgacggtgctgctgactcaggcgttgggcgacacgggggtggaggccccgctgtggagtctgacccaacacgccgtgtccaccgggcccgctgacaccctcctcgcgtccgcatcggcgcaggcactggtgtggggcgtcggccgagtgatcgcactcgagcagcccctgcgctggggtggtctcatcgacctgccgaccgaggtgaacgcgagggcgcgggaacggctggcaagggtcctgtcaggcgtttcgggcgaggaccaggtcgcgatccggacggtgggggccttcggacgcaggctcgtccatgcacccgcgttgcggaccgacctgccgtcctggcagccgagcgggaccgtactggtcaccggaggcactggagcgctgggcggtcatatcgcgcggtggctggcgcatcagggcgcggagcacctcgtgctgaccagccgacgcggtatggccgcgcccggggcgtccgcactcgtggcggacctggaagcggccggagcggcggtgacggtggccgtgtgcgacgtggccgagcgtgcccaactggccgacctggtggcggatgtcggcccgctgacggctgttgtgcacacggccgccctgctggacgacgcgacggtcgagtccctgaccaccgagcaactgcaccgggtgctccgcgtcaaggtcgacggtgcgacgcatctgcacgagttgacccgtgacatggaactctccgcgttcgtgctcttctcctccttgtccgggacggtcggcacaccggggcagggcaactacgcaccgggcaacgccttcctcgacgcgctggccgagtaccgcaggacccaaggcctggtggcgacatcggtggcctggggcctgtgggccggtgacgggatgggagagggcgaagccggcgaggtggcccggcggcatggtgttcccgcgctgtcgccggagctggcggtggccgctctgcgtgcggccgtcgaacagggcgacgcggtggtcacggttgccgacatcgaatgggaacgccattacgccgccttcaccgcgacgcgccccagccccttgctcgccgaccttccagaggtacggcgactcatcgacgcgggcgccgcttcggccgtcgaggagacggaccgggaccgatccggactcagcgggcgcttggcagggctcgacggggccgaacagcggcgactgctgctcgatttggtacgccgcaatgtcgcggtggtgctcgggcacaccgacccagaagccgtgtcgtcccaccgcgccttccaggagctcggcttcgactccgtgacggcggtcgagttccgcaaccggctgggtgccgcgaccggtctgcggctcccggccactgccgtattcgactacccgaccccgctggccctggcggagtacgcgctgtcggaactgctggggacggtcggggagccccttcgcgtcgagtcgagcggctcccccgtggacgacgatccgatcgtgatcgtgggaatgagctgccgcttccccggcggggtgagctcgccggaggacctgtgggacctcctcaccgagggcggggacgcgatgtcggcgttccccggggaccgtggctgggacctggccgggctcttccacagcgaccccggccacccgggtacctcgtacacccggacaggtggtttcctccatgacgcgaccgcgttcgacgccgacttcttcggcatctcgccacgtgaagcgctggcgatggacccgcagcagcggctgctgctggaggcgtcatgggaggcgttcgagcgggcggggatcgatcctcggtcgctgcggggcagcgagaccggggtgttcgccggcaccaatggtcaggactacgtcagccttttgggcggagatcagccgcaggagttcgagggctatgtcggaacgggcaattcggcatcggtgatgtccggccggatcgcctacgtcctgggccttgagggcccggcgctgacggtggatacggcgtgttcgtcgtcgttggtggcgttgcatctggcggtgcaggcgttgcggtcgggtgagtgttcgctggcgctcgcgggcggtgtcacggtgatggcgacaccgggtctgttcgtggagttctcccgtcagcgtggcctggccgccgatggtcggtgcaaggcgttcgcgggggcggctgatggcaccggtttctccgagggtgtggggatgctggtggtggagcggttgtcggatgctgagcggcttgggcatcgggtgttggcggtggtgcggggcagtgcggtgaatcaggatggtgcgtcgaatggtttgacggcgccgaatggtccgtcgcagcagcgggtgatccgtcaggcgttggcgagcgcgggtcttgtggcggtggatgtggatgcggtggaggcgcatggtacgggtacggcgttgggtgatccgattgaggcgcaggcgttgttggccacgtatggtcagggtcgggatgtgggtcggccgttgtggttgggttcggtgaagtcgaatattggtcatacgcaggcggccgcgggtgtggctggtgtgatcaagatggtgatggcgctgcggcatggggtgttgccgcagagtctgcacatcgatgagccgacaccgcatgtggactggtccaccggcgcggtggagctcctgggggagcacacgggctggccggaggtggatcggccgcgtcgggcgggtgtgtcggcgttcggggtgagtgggacgaatgcgcatgtgattgtggagcaggcgcctgaagtggtggagcctgaggctgaaggtgtggtgttgcctgctgtgccgtgggtggtgtcgggtgtgggtgaggtggcggtgcgggcgcaggtggagcggttgcgggcctttgcggaccggaatccgggtctggatccggtggatgtggggtggtctttggcgactggtcgtgcggggttgtcgcatcgtgcggtggtggtgggtgcggatcgtggtgagttgttgggggctttggagggtgtgccggtggtgggtgttccggtggtgggtgggttgggtgtgttgtttgcggggcaggggtcgcagcggttggggatgggtcgtgggttgtatgaggggtatccggtgttcgctgcggtgtgggatgaggtgtgcgcgcagctggaccagcatttggataggccggtgggtgaggtggtgtggggtgatgatgccgagctaattggcgagacggtgtatgcgcaggcggggttgttcgcgcttgaggtggcgctgtatcggctgatcgcttcgtggggtgtgaggggggattatctgctgggtcattcgattggtgagttggctgcggcgtatgtggcgggtgtgtggtcgttggaggatgcggcgagggtggtggtggcgcggggtcgtttgatgcaggcgttgccgtcgggtggtgcgatggttgcggtggccgtttcggagggtgtggtgcggccgctgctgggcgagggtgtggtggttgcggcggtgaatggtcccgagtcggtggtgctgtcgggtgatgaggatgcggttcaggttgtggtggatgtgttggctgggcgtggggtgcggacgcggcggttgcgggtgagtcatgcgttccattcggctcgtatggacgggatgctggcggagttcggtgaggtgcttgggggcgtggagttccgtgccccgagcgtgcccgtggtgtcgaacgtgtccggtgcggtggcgggtgaggagttgtgttcgccggagtattgggtgcggcatgtgcgggagacggtccggttcgccgatgggctggagacgctgcgcgagctgggtgtgggttcgttcctggagttggggcctgacgggacgttgactgccttggcggatggcgatggtgtgcctgtcttgcgtcgggatcgtccggagcctctgaccgctatggcggctttgggcgggctgtacgtccggggtgtccagatcgactggggggcggtgttcccgggtgctcggcgggtcgatttgccgacgtatgccttccagcgtgagcggttctggttggagccatccgctgagcagcctgcgacgagcgtggtggacgcggcgttctgggacgcggtcgagcggggcgatgcggaggctcttgggggcgatgccgagcagtcgttgagtgccgcgttgcctgctttggcgtcgtggcggcgggcgcagcaggaagagtcggttatcgacgggtggcgttaccggctcggctggacgccgatcccggtggtgctgggggagccatgcctcactggcacttggcgggttgtggtcgaaccgggtgcggacggtaccgatgtggctgccgcgctgcggtcggccggggctgatgccgaggtcgtgacgtcggcggaactgagcgcggggccggtcgcgggtgtggtgtcattgttgtcggtcgaggcgacggtggcgctggtgcaggctctcgggacggtcgggatcgatgcgccgttgtggtgtgtgacgcggggtgcggtctccgtggtggacggggatgtggtggaaccgtacgcgtcggccgtctggggtctgggccgtgtgatcggtctggagcatccggaccgttggggcgggctgatcgacctgcccacggaggcggacgcacgtgtgggtgcgttgttggccggggttctcgccgggcgcaccggggaggatcaggtggcaatccgggccgccggggcgtggggtgcccggctgagccgggcgacaccgattgcggacacgtctggcgggtggcgtggtcggggagctgccttgatcaccgggggtacgggtgcgctgggcggccatgtggcgcgctggctggcggggaccggggtggagcgcatcgtgctgacgagccgccgggggatcgagaccccgggtgcggccgagctggtgaccgagttggaggagttcggagtccaggtgacggtggtcgcgtgcgatgtcgccgatcgggaggcggtcgcgacgctgctggtcaccatccccgatctccgggtcgtcgtacacgccgcaggggtgccgagctggagtgcggtggacagcctgacacccgaggagttcgaggagagcgcgcggtcgaaggttgccggggcggcgaacctggacgcgctcctggcggacgctgagctggacgcctttgtgttgttctcgtcggtggcgggggtgtgggggagcggtagtcagtcggcgtatgcggcggcgaacgcgtttctggatgggttggcgtggcggcgtcgtggtgttgggttggtggcgacgtcggtggcgtgggggatgtggggtggcggtggtatggcggttgggggtgaggagtttctggttgagcgtggtgtgtcggggatggctccggggttggcggtggctgcgttgcggcgggcgctgtgtgatggtgagacggcgcttgtggtggcggatgtggattgggagcggttcgggccgaggttcaccgcgttgcgtccgagcccactgctgagcgagctgatccccgatacgtccgaaccactcgcgtcgacggtgggtgagttcgcggtcgagctgcgcggattgtcgcgcgaggaccgggaccgtgccgtcgtggagctcgtacggacacatgccgccgaggtgttgggccaccagaacccgagcgcgatcgacctggaccggacgttccaggagctgggctttgactcgctgaccgccgtggaattgcgggaccggctcggcacggctactcagctgcgattcccagcgtccgtgatcttcgactacccgactccggcggcactcgccgagcatgtgtgcggggcggccctcggactggccgaagagatacaggtagcgcacacgcccagcgcggtggccgacgatccgatcgtgatcatcggcatgagctgccgattcccgggcggtgtggactctccggaggcgctgtggcggctggtcagcgccggtggcgacgccgtatcgtccttcccgtccgaccgtggctgggacctggccggtgtgtacgacgccgacgccactcgctcgggccggtcgtacgtccgcacgggtggattcctccatgacgcggctgagttcgacgccggattcttcgggatctcgccgcgcgaggcgaccgcgatggatccgcagcagcggctgctgctggaggcgtcctgggaggcgttcgagcgggccggaatcccggcctcgacgctcaagggcagccagaccggcgtcttcgtgggcgcgtccgcacagggctatggcggcggggacgggcaggcgccggaaggatccgaaggataccttctgaccggcaacgcgggcagcgtggtgtccggtcgggtggcctatacgtttgggctggagggcccggcggtcaccgtggacacggcgtgctcgtcctcgttggtggcgctgcactgggcggtgcgggcccttcggtcgggcgagtgctccctcgcgctggccggcggagtgacggtgatggcgacacccgccacctttgtggagttctcacgtcagcgtgggctggccgccgatggccgctgcaagtccttcgccgccggtgcggatgggacgggctggtcggagggtgttgggctgttgctggtggagcggttgtcggatgccgagcggaacgggcatccggtgctggccgttgtctccggctctgcggtgaatcaagacggtgcgtcgaatggtttgacggcgccgaatggtccgtcgcagcagcgggtgatccgtcaggcgttggcgaatgcgggtcttgtggcgtcggatgtggatgcggtggaggcgcacggtacgggtacgacgctgggtgatccgatcgaggcgcaggcgttgttggccacgtacggtcagggtcgggatgcgggtcggccgttgtggttggggtcggtgaagtcgaacatcggtcatacgcaggcggctgcgggtgtggctggtgtgatcaagatggtgatggccatgcggcatggggtgttgccgcggacgttgcatgtggatgagccgtcgccgcatgtggattggtctgctggtgcggtggagttgttgacggggcaggtggcgtggccggaggtggatcggccgcgtcgggcgggtgtgtcggcgttcggggtgagtgggacgaatgcgcatgtgattgtggagcaggcgcctgaagtggtggagcctgaggctgaaggtgtggtgttgcctgctgtgccgtgggtggtgtcgggtgtgggtgaggtggcggtgcgggcgcaggtggagcggttgcgggccttcgcggaccggaatccgggtctggatccggtggatgtggggtggtctttggtggccacccggtctgggttgtcgcatcgtgcggtggtggtggttgcggatggtgaggagttgttgggggctttggagggtgttccggtggtgggtgggttgggtgtgttgtttgcgggtcaggggtcgcagcggttggggatgggtcgtgggttgtatgaggggtatccggtgttcgctgcggcgtgggatgaggtgtgcgcccagctggaccagcatctggataggccggtgggtgaggtggtgtggggtgatgatgccgagctaattggcgagacggtgtatgcgcaggcggggttgttcgcgcttgaggtggcgctgtatcggctggtcgcctcgtggggtgtgagggcggattacctgctgggtcattcgattggtgagttggctgcggcgtatgtggcgggtgtgtggtcgttggaggatgcggcgagggtggtggcggcgcggggacgtttgatgcaggcgttgccgtcgggtggcgcgatggtcgcggtggcggcgtcggagggtgaggtgcggccgctgctgggcgagggtgtggtggttgcggcggtgaacggtcccgagtcggtagtggtctcgggtgatgaggatgcggtgcatgccatcgaggagacgttcgccatgggtggggtgcggacgcggcggttgcgggtgagtcatgcgttccattcggctcgtatggacgggatgctcgcggagttcggtgaggtgcttcggggcgtggagttccgtgccccgagcgtgcctgtcgtgtcgaacgtgtccggtgcggtggccggtgaggagctctgctcgccggagtattgggtgcggcatgtgcgggaaacggtccggttcgccgatgggctggatactctccgtgagctgggtgtgggttcgttcctggagttggggccggacgggacgttgaccgccttggcggatggcgatggtgtgcctgtcttgcgtcgggatcgtccggagcccctgaccgctatggcggctctgggcgggctgtacgtccggggtgtggaggtggactgggacgcggtgttccccggcggtcggcgggtcgatctccccacctacgcgttccaacggcagcggttctggttggagtcggcctcggaccagcctgcgaccagcgcggtggacgcggcgttctgggacgcggtcgagcgcggggatgcgcgggcgctgggcattgacgaggaacagccgttgagtgccgtactgcccgccctctcgtcgtggcggagggcgcggcaggagcagtcggtgattgatggctggcgttatcggctcggttggatgccgattccggcggtgttgggggaggtgggcctcatcggtacctggctggttgtggtcgagccgggtgtggacggtactgatgtggccgcagtgttgcggtcggccggggctggtgtcgaggttgtgacgtcggcggagctgagcgctggtccggttgcgggtgtggtgtcgttggtgtcggtcgaggcgacggtgtcgttgctgcaagtccttgtggcggccggggtcgatgcgccgttgtggtgtgtgactcgtggtgcggtctcggtggtcgacggtgacctggtggatcctggccaggcgggaatctggggtctgggccgtgtgatcggtctggagtgtccggaccgttggggcgggctgatcgacttgcctggcgaactggacgatcgcgcggggaatgcgctggtaggcatccttgccgggggcaccggtgaggatcaggtggccatccgtgtcaccggcatatggggtgcccggctggtgcgggcgacgccggtcccgatcggtgacgcgggtggtgaggctgcggccgcgtggcgtgggcgtggtaccgcgctggtcaccggtggtacgggggcgttggggcgtcaggtggcgcggtggctggtgggcagtggtctggagcgggtcgtgctgacgagccgtcggggggttgaggcgcccggtgccgtcgagctggtggctgagttggggagccgagtgcgtgtcgtggcctgtgatgtcggcgatcgtgaggagcttgcggctcttttggtgacgcttccggatgtgcggaccatcgtgcatgcggcgggtgtcctcgacgacggggtgctcgaatcgctgacgcccgagcggatccgtgaggtgatgcgggccaaggccgacggcgcgcggcatctccacgagttgacccgtgacatcgacctcgacgcctttgtgttgttctcctcggctgccgggaccgtgggtaatgcgggtcaggggagctatgcggcggccaacgccgtcctggacgggctggcgtggcgtcgccgggccgagggcttggtggccacatcggtggcctggggagcctgggccgaatccggtatggccgcggagatggcgcggtcgcagggcatggatccgaggtcggcgctcgccgccctggggctggtgctggccgctgacgagaccacggtgatggtggccgacatcgactgggcgaccttcggggcccggttcaccgcctcacggccgagcccgctgctcagcgagttgctcggcgacggatccgtgtcgaccgaggcagccgacggcgaaccggccgacgcgttcgccacccgcctggaggccatggccgagcgggaacgggcggccaccgtgctggacctcgtccgtacgcatgtggccgctgtcctgggacacacggcatccgaggcgatcgacccggcccggcccttccaggagatcggtttcgactcgctcaccgcggtggagctgcggaaccggctcaccgcggccaccggggtacggttcccggcttccgtgatctacgactacccgaccccggccgcgctcgccgagcacgtgtgccgggaggcgctgggtccgggcggacggacaccggctccggtggtgccacgcccggtggacgacgaaccgatcgccatcatcgggatgagctgccgtttccccggcggggtgagctcgccggaggacctgtgggggctgctggccgagggccgtgacgccgtgtcggacttcccggcggaccgtggctggaacctggccgagctgtacgacccggatcccgaccaccccggctcctcgtacgtccgggcgggcggattccttgatgacgcggccgcgttcgaccccggcttcttcgggatatcgccgcgcgaggcgctcgcgatggacccgcagcagcggctattgctggaggtcgcctgggaggcgttcgagcgcgcccatatgtcccccgccaccctcaagggcagccggaccggggtgttcgtcgggaccaacggccaggattacgccgctctggcgagcggggccccgcggagcgcggaagggtatctgggcacgggcagcgccgccagtgtcgcctcgggccggctggcgtacaccttcggcctcgagggcccggcggtcaccgtggacaccgcctgctcgtcgtcgctggtcgcgctgcacctcgccgcacaggccctgcgctccggtgaatgctccttggccttggccggtggtgcgaccgtcatggccactccggcggccttcctggaattctcccgccagcgtgcgttggcggccgatgggcgctgcaaggcgttcgcggcggcggcggacggcaccggctggggcgagggcgtcggcatgctcctggtggagcggctctccgacgcggagcgcaacggccaccgggtgctggcggtgatgcgtggctccgccgtcaatcaggacggcgcgtccaacgggctcacggcgccgaacggcccgtcgcagcagcgagtgatccgtcaggccctggcgaacgcccggctgtccgccacggacatcgacgtggtggaggcgcacggcaccggcaccagtctcggcgacccgatcgaggcgcaggcactgctcgccacgtacggtcagggccggtcccagaacaagccactgtggctcggctcggtgaagtccaacatcgggcacacccaggcggccgccggcgtggccggtgtgatcaagatggtcatggccatgcgacacggtgtactgccgcggaccctgcatgtcgactcgccctcgccccatgtggactgggcggcggcccgggtcgagttgctcgtcgaagcgagggagtggccgcggaccggcgctcctcgccgggcgggtgtgtcctcgttcggggtcagtggcaccaacgcccatgtcatcgtcgagcaggggccggtggtggcccggcccgatcgggagtcggcgcgcgagccgtcaccctccgtgccgtgggtgctgtcaggtgcgggggaggccgggctgagggcccaggtcgagcgcctggcgtccttcatcgacgcccatccgggcctggatcccgccgatgtcgggtggacgctggtggccggccgttcgtgtcagtcgcaccgcgccgtagtggtgggtgcagacctcgcggagcttcgacgtggactggacgcagtctcgaccggtggcgccgcccggtccggccgcaaggtggtgttcgtcttccccggccaggggtcgcagtgggccggaatggcgttggaactgttggagcattcgccggtgttcgcggagcggatgcgtgcatgcgccgatgcgctcaccccgttcgccgagtggtcgctgttcgatgtgctgggtgatgaggtggcgctcggtcgggttgatgtggtgcagccggtgttgtgggcggtgatggtgtcgctggccgagttgtggcgttcgtttggtgtggtgccgtcggcggtggtggggcattcgcagggtgagatcgcggcggcgtgtgtggccgggggtctgtcgctggaggacggtgcccgtgtggtggccttgcggagcagggcgttgctggctctgtcgggtcggggtgggatggtgtccgtaccggtgtccgccgatcggctccgtgaccgtgcggggttgtcggtggcggcggtgaacggtccggcgtcgacggtggtgtcgggggctgttgaggtgctggatggggtgctggcggagtttccggaggccaaacggattccggtggattacgcctcacactccccgcaggtggccgagatccagcgggagctggcggacgtgctggcgccggtccggccgcgcggtggacagatcgcgttccactcgacggtgaccggacggctcaccgacacctccgaactcgacgccgactactggtaccgcaacctccggcacaccgtggaattccagagcaccgtcgaagccctgatgaaccagggccacaccgtgttcgtcgaggtgagcccgcaccccgtgctgaccatcggcatccaggacaccgccgagaccccaggcacccccgacaccccaggcacccccgacaccgcggacgccaccgacgctcacgaggccaccggcgcccccgacgtcgccaacaccgccgacgtcaccggcgctcccgacgtcaccggcgccgacatcgtcatcaccggatcgctgcgccgcgacgacggtggccccgcccgcttcctcaccgccctcggcgacctccacacccggggcgtggacgtggactggagcccggtcttcaccggagcccggacggtggaccttcccacctacgccttccaacgggaacgcttctggctgaagcccgcgcgggcggtgacccaggcgtccgggctgggcctcggcgatatcgagcaccccctgctgggcgcggtactgcccctgcccggggacgagggcggtgtgctgaccggactgctctccctggacggacagccctggctggcccaccacatggtgcgggacacggttgtcttccccggcacgggattcgtcgaactcgccctgcaggccggtcagcacttcggccactcggtgatcgaggagctgaccctgcatgccccgctggtggtgccggaccagggcggggtccaggtacaggtggccgtatcggcggcggacgaacggggccggaggccggtcacggtgcactcgtgccgtgccggggagtggctgctgcacgcctcgggcactctcggcgccaccggaggcctcgacgtcaccgagccgcgccccgccgacgtggcccggcccctggaggtctggccgcccgagggcgcgcggagcctcgatgtctcggggatgtacgaggcgatggcggagcgcggctacgggtacggtcccgctttccaagggctgcgcgccgcgtggacacgggacgatgagatctacgccgaagtggctctggagccggaggcacaggacgtggcggcgcggtgcggtgcgcatccggccctgctcgacgccgcgctccacggagtggggctcggccgcttcctcaccgaccccggccaggcgtatctgccgttctcctggagcggggtcgcgctgcacgcggtaggcgcctccgccatccgcgtggtgctctccccggccggtacggacgcggtgtcgctggaggtgacggacccgacgggagcgccggtgctgtcggtggcgtcgctctcgttgcgtccgctgtccagcgggcggatcgcggacacccgaggggtggaccaggactcgctgtaccgcgtggactgggtcgagatgccgctgccgactgccccggcaggctcggccccggccgagtacgacgcgccggcgatgttcgacgccctggtattcgacgccccggtcgagtacgacgttctcgcctccgacgcctccgacgcctccgacgcctccgacgcccccggcacccccgacgcctccagtgccccggtgcccgacatgcccgacatggtggtgctgccgtgtgagtcggccggtgacgcggtgtccaccgtcgtgtgccgggcgctggcggcggtacggcgatggctcgccgacgagcgctgtgcccggtcgcggctggccgtgctgacgcgcggcgcgatggccaccgctcccggcgagagcgtcgaagacctcggcgcggcagcggtctggggcctgctccgcagcgcccaggccgagcacccggaccgcttcgtcctcgtcgaccacgacggccaccaggattcccgtgcggtgctcgccgccgcgctggccgccgcggtcgacggtggccatgcgcatctcgcgctgcgccgtggccgtgtcctgacgcctcagctcgctccgctcaccccgtccgcgaccgccctgtccaccaccgcaccgcccgccgccaccccaaccccggaggccggggcaccgtggcggatggacgtcaccagtcagggcacgctggagaacctggccgccgtcccctgcccggaggccgccggtgtcctcggcgccggacaggtgcgggtggcgatgcacgcggccggggtgaacttccgggacgtcgtcgtcgccctcggcatgatccccggtcaggacgtcatcggcagcgagggtgccggagtggtgctcgacatcggccccggtgtgtccggcctggcgcccggtgaccgggtgatgggtctgttctccggggcgttcggccccgtggcggtgaccgatcaccgactgttggcgcggctgccggaaggctggtcgttcgccgacgccgcggccacgccggtggtgttcctgaccgccatgtacgggctgatggacctggccggtctgcgacccggtgaatcggtgctgctgcactcggccgccggcggggtgggcatggccgcgacacaggtggcccgctggctcggcgctgaggtgtacgccaccgcgagcccagggaagtgggacgcgctgcgcgccggaggagtggcggacgaccggatcgcctcgtcccgctccttggagttcgccgaccgcttcggccgggtggacgtggtgctgaactcgctggcgggcgagtacgtggacgcctcgctcggcctgctcgccgacggtggccgtttcctggagatgggcaagaccgacatccgcgacggtgagcgcgtggccgcggagcacggggtgcggtaccaggcgttcgacctcatggacgcggggcccgaccgggtcggggaactgctcaggctgctggtgtcgctcttcgagcgagggatcttcacggcactgccgacccgcgtctgggacgtccggcaggcgggtgacgcgctgcgcttcctctcgcaggcacgccacatcggcaagctggtgctgtccattccgcagccgctgcgggagggggacaccgtgctcatcaccggcggcaccggcacactgggcgggctggtcgcccgtcacctggtcgaacggcacggagtacgggatgtcgtcctggccggccggcgggggccggacgccccggacgcggccgaactcgccgccgccctgcgcgaatacggcgcccgggtgcgggtggtggcctgtgacgtggccgaccgggaccagctggcacggctgctggacaccgtctccggcctgcggatggtggtgcacaccgcgggtgtgctcgacgacggggtgatcgagtcgctcaccccggagcgggtgcgcgaggtcctgaggccgaaagtggacgccgcctggtatctgcacgagctgacggccggtcgtgagctggcggaattcgtggtgttctcctcggccgcgggtgttctgggaagccccgggcagggcgcctacgcggcggcgaacgcctggctggacgcgctgatggcgcatcgccgggccgccgggctgccgggtctctccgtggcctgggggctgtgggccgagcgcagcgggatgaccggccatctgtcggaccgggatctcgcccggatggccagggccggtgccacgcctctcgccaccgatcaggggctccggctcctggacagtgccagggcggccaccgaggcgctcgtgctggccacaccgctggacgccgcggcgctgcgggcacaagccgacgccggggcgctgcccgcgctcttccgcggtctggtccgtgcgccgatccgccgcgcgaccggcgcgggcccggtggaggacgagtcgtcgctgcggggccggatggccgcgatgccggtcgccgagcgcgaacagctggtgctggacctggtccgtacgcaggtggcgaccgtgctggggcacggcaccgccaccgcggtcgacccggcgcgtacgttcgcggagaccggcttcgactcgctcacggccgtcgagctgcgcaaccggctgcgcaccgccaccggggtcaggctgtcggccaccgcgatcttcgactatccgacacccgcggtcctggccggtcatctcctccgggagctggacggcaccgtcggcgaggccgtgacacggcccgccgccccggccgccgccaccgaccgggacccgatcgtgatcgtcggaatggcctgccgctatccgggcggagtggcgtcgcccgaggagttgtgggagctgctcgccaccgggcgcgacgcggtcgcggatctgccggacgaccggggctgggacctggacggcctgtacagcgccgatccggacagctcgggcacctcgtacgtccgctccggtggcttcgtgtacgacgcgggcgagttcgacgccgacttcttcggcatctcgccgcgcgaggcgctcgcgatggatccgcagcagcggttgctgctggaagtggcctgggagacggtggagcgggccggtgtcccggcggcgtcgctgaaggggagccagaccggggtgttcgtcggtgccgcggcacagggctacggcacgggggccgggcaggcggcggagggatccgagggctacttcctgaccggtggcgcgggcagcgtggtctccggccggctctcgtacaccttcggcctggaggggccggcggtcaccgtggacaccgcctgctcgtcgtcgctggtcgcgctgcacctggcggcgcaggccctgcggtccggcgagtgctcgctggcactggccggcggggtgacggtgatggccaccccgggcatcttcgtggagttctcccgacagcgcggactggccgccgacggccgctgcaaggcgttcgccgacgcggcggacggcaccggctggggcgagggcgtcggcatgctgctgctggagcggctgtccgacgcccgccgcaacggccaccgggtcctggcggtcgtacggggctccgccgtcaaccaggacggcgcctcgaacggcctgacggcgccgaacgggccctcgcagcagcgggtgatccgggccgcgctggcgaacgccgggctggccgcgtcggacgtggacgcggtggaggcacacggcaccggcaccagcctgggcgacccgatcgaggcacaggcgctgctggccacctacgggcagcaacgcgaacggccgctgctgctgggctcgatcaagtcgaacatcgggcacacccagtcggccgcgggagtggccggtgtgatcaagatggtgctggcgatgcggcacggggcgctgccccgcaccctgcacgtggaccagccgtcgacccatgtggactggtcggccggtgcggtggagctgctgaccgagcccgccgagtggccggggacctcccgcccccgccgggccggggtgtcctcgttcggggtgagcgggaccaacgcccatgtgatcctcgaacagccacccgcggaggcggagtccgggcccgctccggagtcggcacccgggcccgtcccggcggtggtgcccgggcccgtcccggcggtggtgccatgggtgctctccggccagggcgagcgcggactgcgggcgcaggccgcccggttgcggtccttcctggccgcgcgccccgagtccggcccggccgacgtgggctggtcgctggccgccacccgttcggcgctctcccaccgggccgcggtggtcggggcggaccgggcggaactgctggacggactggccgcgcttgcggccggcgagcccgccccgggcgtggtcttgggcaccgcggacccgggccgggtgggcgtgctgttcgcgggccagggtacgcaacggcccggtatggggcgtgagttgtaccagtcgttcccggttttcgcggcggcgtgggacgaggtgtgcgccgcgctcgacccgcatctggaccgtccgctcggcgaggtggtgaccgatgccaccggcgcgctggacgccaccacgtacacgcaggcgggcctgttcgccctcgaagtgtcgctgttccggctggtgtcctcctggggcgtgcggccggactatctgctgggccactccatcggcgagctggcggccgcgcaggtggccggtctgtggtcgctggaggacgccgccaaggtggtggcggcccggggccggctcatgggcgcgctgccgccgggcggggcgatggtggccctggccgcgccggaggaccaggtacggccgttcctgaccgaccgggtcgccctcgcggccgtgaacgggccgtcgtcggtcgtggtgtccggggacgaggacgcggtgtgcggtgtggccgaggcgttcgccgcccgtggggtgaagacgcggcggctgcgggtcggccacgccttccactcgccgctgatggacgagatgctcatcgcgttcgccgaggtactcgacacggtggacttccgcaccccgcggataccggtggtgtcgaacctctccggtgcggtggcgggggaggagctgtgctcccccgcttactgggtgcggcaggtgcgggagacggtgcggttcgccgccgggcttgagcgtctgcgggagctcggcacgggcaccttcctcgaactcgggccggacggcaccctcaccgccttggcccaggcccagatcaccggggcggacgccgagttcatccccactctgcgcgccgaccggcccgagccggtcacggtcaccaccgccctcgcccagttgcacacacacggtgtggagccggactggtccgcggtcttccccggcgcccaccgggccgagctgccgacctacgccttccagcgctcccgcttctggctggagccctcccgtacacccggtgacgcgggcgacttcgggctcggcgcgctggaccatccgctggtcggcgcgagggtgccgctgcccgacgcggacggcgttctgctcaccggccgcatctccgccgaggcccactcgtggctgatcggtcagcgggcgctgggcgtgcccctgttcccggcgaccggcttcctggaactggtgctccaggcggggctccagtgcgacagccggacggtggacgaactcaccatccatgaaccactcgtcctccccgagcggggcggggtcgaggtgcaggtgtccgtccgtggcgccgacgagtccggccgccgcccggccaccgtgtactgccgccgcgaccagcggtgggtccggcatgccacggccgtcctcggcgcggaccggccgcccgcgccggagccgcgccccgagccctggccgcccaccggcgcccggccgctggagtccggcgggacaccggcgtggcgccgtgacgacgaggtcttcctggacatcgagctgcccgaggtggccggggccgaggccgaacgctggacgctgcatcccgccctgctcgaacaggcgttgcgcggggaggcgctggcagggctggtcacggcggccgaggggacccatctgccgttctcctggacggggatcaccctgcacacgacgggtgccacgagactgcgagccaccctcgcgcccgtcggcccggacacggtctcgctccacgtggccgacgccgccggaacacccgtgctgtcggtggactcgctggcgctccgcccggtgtccggacagcggctgcgccaggccaacgcggcgctgttccggccggtgtgggcggcttgccgcacgcgggccgaaccggacaccggctctgtccgatgggggctcgtcggcgacccggacgcctggaaaccggacacgctcggcgcgccggtcgcgctgtacccggacctgtcggccatcgaggacgtaccggacgtcatcctcctcccgtgcgtatccgagggcggaacggcgtccgaggtggccgtccgcgtatccgagaccgtgcggacgtggttggccggggagcggttcgccgcctcgcgtctggtgctggtgacccggggcgcgctcgccacggcggccggtgaggagctcgaggacctggccgcggccgcggtgtggtcgctggtcgagcccctccaggcggccgtggcgggacggctgacactcgtcgacaccgatacgtccgatctgcgcatgctgcccgccgcggtggccgtgggggaggaccgggtcgcggtccgggcgggagcggtgctggtaccggacctggtcacgccgccggccaccgagcaggatccgcccgcctggggcccggggacggtgctggtcaccggtgggtcggccatggctgtctcccggcatctggtcgccgaacgcggtgtgcgtgacctggtcctggccggggacggcgacatggccgaactggcggccctcggagccacggttcggctcgccccgtgtgatccggcggacggtcaggcgctggcggcgctggtggcggagattcccgggctgcggagcgtggtgcacaccgcggccgacgccccggagcggacccggtccctcttgccggaatccctgcggccacagctgcggtcgggagtggcggcggcctggaacctgcacctggccacgcggggcctggaactggaccgctttgtgctgttcacctccgccgacgggacactgggccccgcgtacgccgacgcgctggccgcacaccggcgggctcgcggactgcccgcggtgtccgtctccaccgatctgggtctcgccctgttcgacgaggcatgcgccgggcccggggaggcgatccgggtcaccaccgccacgccggcccccgcacccaccgaggcggaccggcagccggtggaacaacccccggcggccgaggcctccgcgaccacgttgctggagcggctggccgggcggacggaggacgagcaggacgagatcctgctggagctggtccgtggccaggtcgccatggtgctcggccatcccgacgccaccatggtcgacccggaccgaggcttcgtggaactgggcttcgactcggtggcggccgtgaagctccgcaaccaactggccggagccacccggctcgacctgcccgccagcctcaccttcgaccaccccacggctgtcgatctcgcccgccatctgcgcgccgaaatgctgcccgacgacgcggcggccgccattctcgtgctcgaagagctcaacaagctcgacgattcgatcctcgtgctcgacccggcaagcgcggcacgggtgcggatctcgaccctgctccaggacctggccgcgaaatgggtcgagcggacggatcggccatgaccacacacgatcagttgatgcgcgaacgagggagtcaacagtgagcgagaccttgtcccttcccgggaccgtgaaggccgaacggcgttgtccgtacgacccgccggaggcgcaccgccgactgcgggacaagggcgaactgggcaaactggagctgcccggcggtctggtgatgtggttcctgaccaagcacgacgacatcagggccatgctggccgactcccggttcagcggtgcgagggtgccgtttccggcgatgaacccggagatacccgcgggcttcttcttctccatggacccgccggaccacacccgctaccgccgcacactcaccgccgagttctcggtgcgcggcgcacgcgaactgaccggccggatcgagcggctggccgaccggcacctcgatgcgatggaggcggcgggcacgagcgcggacctcgtggcggcctacgccagtccggtgcccgcgatggtgatctccgaaatcctcggcgtgccgtacacctaccaccagaagttcgaccacgaggtacgcacgctccgggagaccggcggcgacgatcaggccgtcggcgcgatggcgaccgcgtggtgggacgagatgcgcggattcgtgcgtgccaaacgggccgagcccggggacgacatgatcagcaggctgctgcatgatgaggtcgagggcggtgcgctgaccgacgaggaggtggtcggcattgcgatgaccatcattttcgccggtcatgaacccgtggagaacctgatcggcctcggcatgctggcgctgttccaggacggtgagcagctgacccggttgcgggagaaccccgacctcattgacagcgccgtggaggagttccttcgctacttccccgtcaacaacttcggcaccgtgcgcaccgccaccgaggatgcagtgatcaatggtcaccccatcgcgaagggcgagatcgtggccggtctggtgtccaccgccaaccgggaccccgagcggttcgccgatcccgaccgccttgtcctcgaccggtcgcacacctcccacctcgcgttcgggcacggtgtgcaccagtgtctgggccagcagctggcgagggtggaactgaaggtgctcctacagcggctgctcgtcaggttccccgctctgcggctggcggtggccccggaggagatcaggtaccgggagaacacctcgttctacggtgtccacgagctcccggtgacctgggcggccgagtagccgcagccggggccggaagacacggcgcgggcggtggccgcggggtccggcgcgagcggtggccggatacccggccaccggctcagccggcccgggtgacgcccactgccgccctgagatccgcccagaactccggccgaccgcggatctcaagagccgagccggccgccggtcaggcgtgccagcgggcggcggcgacgagccgcgcgcctgcgcaggacgaccgcggcggtggcgcccgccgcggcggcacccgcaccggcgacggctgccaggaccttccgggccttgatcatcatccaggccgaacccgccgccgcggcagccttcttgggcgcctcagcggccacggtacgaccggtggtcacggccttcccggcggccacttgagccttgtcggcggcgacatgagcggtgtgcgccgcggtcgtcgccgcgccggccgcggcggtcttcgccgtggtctccgccttgcccgcggtgtccttggccctttccgcggtctccttggccttggtggcggtggccttcgcgttcacgttggtgctccgggtagtgtgtgcgttcttcttctcggtcatgttcaccgcgttaccactcgacccgacaacaaacccgcgtcctcggggccggccgccacggcgtgacgatccgagggggcggcacaccgggaggcgcgccgcccatcggctcattcgatgtctgagccgcccgaaccacccgagccgcgccgctgctggaacagcacaaaccctccggccagggcgaacagacagacgccgatgatgatgcccacggcgggccaagcggatccgccgtcggatgtcgcggccttcgaaggctccagcgatgtggcatcgggcaactgtcttacgacgaaactgtgttcggcgttctcgccgggtgcgagcgccgggccgccgaccgagtagccgtcatcggtgggcttcagcttccagcccttcggggcctgcttcagcctcacatcgccggggtcgatgcccgtgggcaacacggtccggatctcggtgaaaccggccctcccgtcctccgcctccgactcgaacgtcagcgtgacgtccttcgccagggcgcgggagtcggaggcgctcacctcggtgtgggccagggcgggggtcgccgtggcgaggacgagcgccgaggtggcgacggccagggcgccgatccgtcgcggccgcgggtgggacggcggacgatgtgctttcacggtatctctcctcgtccatggggtggtcacccgagccctcctcaccggtcacccggcgcgctcaccaggtgcgttcacattccccggtacgtacggcccgggcgcgatgttcatcctcgcccagaccggaagcccgcttgccacgcgggggagcaaggagttccggcgtcttcgtggcccgcgcaaggcggaccgagggggtcgccgcgtcccagtgcggtgatgtgcccggtggtcaggtggtccgctggtcccgcagggtccgggacagctcctcctcggtgagcacccggggcgcgggtccggcaccgggcttcgtctcggcgccgttcgccgggctcttgttctcggtcgtcgtcatgagggtgcacctttcgctggtggtggcggaggacgggatcaggcggtggcgaccggtgtggcgggcggattggcgttccgcggcacagcggggggcttgcgcgcaggtcctcgcagtacggtgaacgccaccgcgaaggccgccacgatgagccccgttccgacggcgaaggccaggtggtagccgccggtcagcgcctcggcccgacccttgccccgggagagcagggcatccgtgcgggaggcggccagggtggacagcaccgcgacgcccagcgccatgccgatctgctgggtggtgttgaacagcccggagacgagcccggcctcgtcctccttcgcaccggacattcccaggctggtcagcgcagggagcgccagcccgaaaccggcggcgagcagcatcaccgggaggaggtcggggaggtaccgggcgtgcacggggacgcggacgagcaggccgagaacgccggtcaggagggccagcccggtcagcagcaccgcgcggtcgccgaagcgtgcgctgagccgtgcggagacgccgagggacaccgcgccgatggcgatggcggccgggagcatggccagaccggttccggtggcgtcgtaccccagcacattgcgcagatagagggcgaccaggatctggaacgagaagagcgcggccaccatcaggagctggaccagattggcccccgccaccccgcgcgaccgcaggatccgcaggggcatcagcggggtgcgggcggtggtctggcggaccaggaacagggcgatcaggaggatcgagacggcgccgaggccgagtgtgcgcgccgccgtccagccgtagtccgccaccttgaccacggtgtagatgcccagcatcagcccggtcgtgaccagcagggcgccgaggacatcggcgccggccgcgaggcccggcccgcggtcggcgggcaggacgggtatggcgaccgcgagcgtcagcagcccgatcggcagattgatcaggaagatccagtgccagctgagcgcgtcggtgaggaggccgccgagcacctggccgatcgacgctccggcggcgccggtgaagctgaacacggcgatcgccttcgaccgttcggcgcgttcggtgaagagcgtgacgaggatgcccaggctgaccgccgaggccatcgcgctgccgaccccctggaggaaccgtgcggcgatcagcacagcgggggaggtggccacggccgcgagcaacgaggccgcggtgaacaccgcggtaccggtcaggaacacccgcttgcggccgatgagatcgccgatacggccgccgagcagcagcagaccgccgaacgcgatcaggtaggcgttgacgacccagctgagcccggcgggggagaaccgcagatcgctctggatggcgggcatggccacggtcacgatgctgccgtcgaggatcaccatcagcatgccggtggcgatgaccccgagggccagtcgacgtgtcggggggacacggggagacgtcgggtcggaagaggcggcggacatgcggacactcctgtcagtaggcgcgacaggagtgaccgtagcagatggttttgttgcagactatttgtttcggctctacttgtggcgcatgacgggcggccgcgcggatgtctccgctctacttctcgcgctgccgcgccctccgcgccgggcgggggctctcggcgggcgtggccagatgcccctcggacagccgggtcaacgcctttagcagcgcggcgcgttgggtctcggggagtgtcgccaacgcctcgcgatggacgcggtccacgatctcctggctccgttcggcgatccgcgcgccctcctcggtgaccgcgatgatccgggcccggcgatcgtgggtcgaggcgcgccgctccgcgaggcccgccttctccagggcgtccaccgtcaccaccatcgtggtcttgtccatgtcgccgatctcggcgagctgggcctgggtgcgctcttcctccagggcgtggaccagtacgcagtgcatccgcgccgtcagcccgatttcggcgagcgcggccgacatctgggtgcggaggacgtggctggtgtggtcgaggaggaacgacaggtcgggttcggtcttggtgggcgccatggcggtcatgcggcccagggtaacaattcgatccgtactggattatccggaacagtccatagggaggggtggggtcaggggtcagccgttgcggtagagggcggtgagcagctcgaccgcggtctgggtggcggggtgcgggtcgtcccgccaccaggcgaggcgtaccgcgatgggctcggcgtcgcggaccggccggtaggcgattccgggcctcggatactggttggccgtggactccgccgtcatgccgacgcagcggcccgcggagatcacggtgagccagtcctccacgtcgtgggtctcctccgtggccggccgggagtcgggcggccacagctccgtggtggtggtaccggtcctgcggtcgaccagcagggtgcgcccgctgaggtcggccagccggaccgagcggcgcctggcgagcgggtcgtcggcggccacggcgcacagccgccgctccagtccgacgatggcggagtcgaagcggcgctcgtcgagcggtctgcgcaccacggccaggtcgcaggcgccctccgtcagccccgcggtggcggaattgacgcggacgaggtgcagctccgtctcgggatacgcctgcgcccagcggcgctggaaggcgggggtgtgacggcccagcgcggaccaggcgtagccgatccgcagatgggcgtggcccgatacggcctcccggatcagcccgtccacctcggccagcacccgccgggcgtgtgccaccacccgcagcccggtgcccgtcggggtcacctcgcgggaggtccgccgcaacagccttgtccccagggcgcgttcgagcgctgccagggtgcgggacacggccgcctgggagacgccgagcgcgatggcggcgtcggtgaaggtgccctcgtcgacgatcgcgacgaggcagcgcagttgccgtagctccacatccatacgtccagcgtatagatagaaccccgaacgcattttgcgcatgcatgagccgggcgcacgatcgacgcatgcgactcgcccccgcgtcacgcaccccgtccccacgagcgatggacaccgcacaccggaccgcgccgacccctgccgactacgacctgggacagggcctggagcggggcctggcccctgaccctgatcagcggccgaccggacggcggttcgccggtgtggccacgatgatcggcagtgggctgtccaaccagaccggcgccgcgatcggatcccaggccttccccgtcatcggcccggtcggggtcgtcgccgtccgccagtacgtggccgcgatcgtcctgctggccgtcggcaggccccggttgcggagcttcacctggtggcagtggcggccggtggtggggctcgccgtggtgttcggcaccatgaatctgtccctgtacagcgccatcgaccgcatcggcctcgggctggcggtgaccctggagttcctcggcccgctgtgcatcgcgctcgccggctcacggcgccgcgtggacgcctgctgtgcgctggtcgcggcggccgccgtggtgaccctcatgcgcccgcgcccctcggccgactatctgggtatggggctggggttgctggccgccgtgtgctgggcgtcgtacatcctgctcaaccgcaccgtggggcggcgggtccccggcgcccaggggtcggcggcggccgcggggatctccgcgctgatgttcctgccggtcgggatcgccgtcgccgtccaccagccgccgaccgtgagcgccgcggcgtacgccatcatcgcgggcgtcctctcctcggccgtgccgtacctcgcggacctgttcacgctgcgccgcgtgcccgcccaggcgttcgggctcttcatgagcgtcaaccccgtcctcgccgcactggtcggctgggtcggcctggggcagagcctggggtggacggagtggatcagcgtgggcgccatcgtcgcggccaacgcgctgagcatcctcacccggcgcggctgaaggaccagcgggggtggcccggtgacttggctgacctggacccgggggtggacccggggacggagggccgcgccgcccccaggccaccgctccgcccccgggccaccgctcagcccgcggcctcgaacagcgcctccgcggcggcgatcgcctcggccagggcggtgggctccggccgcagccccgccacgatcgtgtcgatggcgcgcaggtccgcccgctggaggagccgcttctcgttggtgacccactcaccgcgcgccgccagcaccgcgtgcgccgtctggagggcggcggtcgccaccgcccccgcgacctcggtggcctggccacgtcccacgtacgcggccgaggcgtaccgcagggtcaaggccgcccgcccgcgccacgccgggggtgccgcctcacgcagcgccgccgggtactcggggcggggcagggtgccccgcagcacctggttcagggcgagttcggccaccaccagatagctggggatgcccgcgaggtggaacatcagcggctcccagtggaagcggccccgtcgcgactcggcgagttcgtgttccaccacctcgaggtcgcggtagtggacgtccacgcgccgtccgtcgatcgtcagccaggcgcccccgttgaagacaccaccgccccactcgccgagctcggagacctcgccctcccagcccacggcccgcagcgcggccgggtcgaagccgcctcggtagtacagggccaggtcccagtcgctctccggggtgtgggtcccctgcgcacgcgagcccccgagggcgacggcgtgcacggcgggcagggcggcgagtcgctctgcgacatcgtcgaggaacgtgtcgtcggtcatgaggaacatgtcgtcggtcatacggatccgatcgtgtgaagtggatgacgggtgccgcgggcacaccgaacgcacgccggagcacaggctcgaacggcggcgtccatacggatcggtgtgccgggtcatcactccatgacgccgaccttaccgcccccgtcagagggcggcagcggccagcggcggatcagtccttcaccaggcccaggcggaacaggctctccgcggtgtcgaggatggtcgtcaccgggtcgcgcggggtccagccgaacacggaacgcgccttctcggtgcgcaggatcggcacccgctccgtcacgccgaccgcttcccgcgctcgttcgtcgtcgaactcccgcgtgggcacccgggcggcgcgctcgccgaggtgctcggccagcacctgggcgatccacaggaagctgacggtccggtcgccgctggcgaggaagcgctctccggccgcggcggggtgtgccatggcccggaggtggagctcggcgacatcgcgcacgtccaccatgccgaagtgtgcgcgggggacggccgacatcgccccctccagcatcgcccggacgtgttccgtcgaggcggacagccgggggccgagtgccggaccgaagatcccggtcgggttgatcaccgtcagttcgaggccgtccccctccttcgccacgaagtcccaggcggccagctccgcgatggtcttcgagcggatgtagggcgggttgtcgtcctcggggtcggtccagtcgctctcgtcgtactcgtcaccgtccttgtggctgtatcccaccgcggcgaacgaggacgtcatcacgacccgtttcacaccctggtcccgtgcggccctcagcacacgaagggtgccgtcccgcgcggggacgatcagctcgtcggcgttgtccggctggacggcggggaacggtgacgcgacgtggtggacgcgggtgcaccccgccatcgcgtcgtcccagccgtcgtccgtggtcaggtcggcgctgacgatatcgagccgcccgccgggatcgacaccggaggccgcgatggccgaccggacactcgcggcggcgccggtggccgggccgtgtgaacggaccgtggtgcggacccgatggccgctccgcagcaggccgctgatcacatgggtgccgagatagccgctgcctcctgtcaccaggacgagttcgccactaacggtgtcgccacgggcgtcggcgccggcatcagcggacacgggggttgccttgctttccatggggtacttcggatcccttcccaagtgtgtttctcgcagctgtgtctctcacggccgcagcgcgtcgatgacgtccgtcagttcgtcgatcgcccggcgctccgcatcggcgtcggcgcgctcccgcgcgtcccacatgtccgccttgagccgcagatagcgcagccggacctccatgagcgcgatctcccgctccaggcggtccgcgttgcgttggaagaggtcacgcaggggagccgcaccctgatcgccttcgtcgaggtggccgaggtaggcgcgcatgtcctgcatgctcatgccggtggatctcaggcaccccagcgacctgatcgtctccaccacggaggggggatagcgccggtggccactgtcccggtcgcggtccacggcggggatcaagccgatcttctcgtagtagcgcagtgtcggctccgagaggccactcagcctcgacacctgctggatggtcatcggggagccccggacctctgtcctcgtcgttgtcataggacgagcatccgatacttgaagcgcttgaggtcaagcgagccgatcggcctttgcggggacggcggtcccggaagtgggcgcgcccgggcgcttcccggccgtggcgatggagatgtcccgcatgaggagcagcgccccgagggcgaggagcccggcggcgccgccgctccaggagacggtggagccgatggccgtggcggtgggcgcggccgcgccggagtggccgatcagggactgggcgctggccaggccgaccgcgccaccgagctgcttggtgagcgcggaacccgcggtggcggtgcccatgtccgcacgcgggacggcgctctgggtggcgatggtgagcccgcccatggccggtcccgcgccgagcccgacgagcagcagcagaacggacgtcagcgcgagaggggtcgtggcccgcagggcgacgaaggcggcggtaccggcggtgagcagccccgcgccgatcagcaggaccggcttgacgtgcccgctgcgcagcacggtggcggcggtgagccggttgcccagggtcatgccgatgagcagggggagcagcagcagaccggaggcggtggccgaatggccgcggatgtgctggaagtacagcggcaggaagattcccaccggcgccgcggcgacctggaagaagaaaccggcggtcagcagggcggtgtaggtgcggtgccggaacagccgcaggggcaggacggggacggcggcccgccgctcgaccggtatgagcgtggtgagcagcgccagaccgccgagcagacagcccagcacggccgggtccgtccaggagggcgcgtgtccggcggtcgcgttccccttgaggctgaggccggtcagcgcgagggcgagccccgcggcgagcaggaggatccccgccacgtcgagccggccggacggcggggtggcgggacggcggtcgggcagggccaggacgatgacggcgcccgcggccagcccgagcgggaggttgagccagaacgcccagcgccagccgatgtgatcggcgagtaacccgccgaggagcgggccgcccaccatgcccaggatcatcatggcggccatcgccgtctgcatccggatgaggccctgggggcgggacggcgggtggaggtcgcggaccagtgccatgccgagggtcagcagggatccggcacccaggccctggagcgcgcgggagaggatcagggcgggcatcgaggcggacaggccgcaggcgatggagccgatcaggaagacgccgagcccgccgatcagcagccggcggcggccgtggaggtcggagaagcggccgtagaccggcacgctgaccgaggaggtcagcagataggcggtgacgagccagacgtaccaggagtcccctccgccgatctgctcgacgatgcggggcagcgcggtgccgaccacggtgccgtccagcatggccaggaaggcgcagcccagcagggcgatggtgaccagggcccggcggcggtgcgggagcgcttcgtacccgtcgggtccggtcaccgggcctccccgggcgccacgagcgcgccgtgcaggaagaggtccacgacttcctcggtggtcagcggctcgggggcgcccaggcgcccggccgacatcagcgtcagctggaaggcgtcggcgagccgttccggggcgagtcgcaggcggtcccggtcgggctcgaacagcgcggccagcgcggcgcgcggccggaccaggctcgcctcgcggtccgggaggcgcccgtccttgccgggcttgggcgccatgcgctccagccgcccggccgccgcgagcgccccggcgaccgcgccgatgcgcgccatgtgtccgcgcaccacatcggccgcctcggcgagccggtccgcaagcggctggtcaagggcgatcgactccagatgggccacggtgtcatcgggccgcacggcctccgccatacaggccgcgagcagggcgtccttgtcctcgaagacgcggaagatagtgccttccccgatgcccgcggcccgggcgatcttcgcggtcgtcacggtggcgccgtattcgacgacgagggggagcgcggcggcgacgatcatcgcgcggcgctggtcgggatccatggccggagcgcggcggcgggtcggagtggaggggttctctgccttctctgtcatgcgggatacggtacggagtgagtactcactccgtcaatgcacggtgcgcggccacaaggcgagtggcggttcggcttcgacgttgtcggtcagcgcgcggcgagcagggcccggcgcagggcgcggcccgcctcggacgggggatggttgcgcgaggcggcgaggccgagaccgtgcagcggcggtggatcggcgagatcgacctgggtcaggccgggccgcgaggcgatggtctcctcggccacgaaggcgagcccgagacgccgccggaccatggtcagggcggtcgtggtgtcgacgacctccagtgccacggtgcgctgaactccggcggtgccgaagaggctgtcgacgatggtgcggtcgccccaccccgtggggaagtcgatgaagcggcggtcggcgaggtcggcgtaggtcacgccgtgcgcctcggcgagggggtcgtcggtgcggcaggccagcccgaggcgtatccgcgacacatcatcgatgatcagatccgggccgaggacggcggggccgtgcgggggcaccggcagcagcatcaggtcgaacctgccctcgcgcagggcggtggcgtgtccggccagcggaccggtcgagtggcgcagccgcaccacgacatcggggtgctcggcctgaaacgtgctcagcgccccgatcaggtcgaacgagccggtggacaggaccgtcccgagggtgaccgtaccgctgagccccccggtgagacggcccatgtcatcgcgcgcccgctgcgcctccgcgagcaggatccgggcccgggccagcagggtgcgccccgcggtggtcagctccagggtgcggtgcgagcggtcgaagagcgcggtctggaactcctgctccagccgggccacggcggcggaggccgccgactggacgacgtgttcccgctgggcgccgcgggtgaagctgcgctcctcggccaccgcgacgaagtacgccagctgccggagctccaccatcatctccattcgcgatgccacacagcacacatcatcgttggacacgatacctatgggaccgccaccgtggaggggaagcggaacgccccggccggacggcccggttcggcgccacgccccccaacttccccgtgtgccagcacacttcaccacggaaggcatccatcgtcatgagcgtctcagccatccagatcgggctccaccccgatgccatcgactacgaggcgccggagttcgccgccttcgccggtctgagccgggagacgttgcgcgccgccaacgacgacaacctcgccctgctgctcgacgccggatacgaggcggacggctgtcagatcgacttcggggagaccgccctcgacaccatccgcgccatgctcggccgcaagcgctacgacgcggtcctcatcggcgccggggtacggctcaccgcgggcaatacactgctcttcgaatccatcgtcaacctcgtccacaccgcgttgccccacgcgcggttcatcttcaaccactccgccgcggccacccccgacgacatccgccgccactaccccgacccggcctccaccgttcccctcgacgtcccccgcgacctcgaggaggccgcgctgaagaaccccggcaacgccgcccgccccgaagccgcccacggcccgcgggagacgcggtgaccgccccggcccggccccacggtgaggcgaacccggaccttcacaccacggatgtgctcgtcgtcggcggcgggccgaccggaatgaccctggccggggatctggcacgggccggacgcgcggtcaccgtgctggaacgccggccggcgatccatccgtccagccgtgccttcgtcaccatgccccgcaccctggaagtcctcgacagccgtggtctggccgacgacctcctggccggggcgaacaccaccgaagcggtccacctgttcgcgggcgccacgctcgatctgacacatctgccctcccgccaccgatacgggatgatcaccccgcagaccaatgtggaccaggcgctcgaacgctacgcccgcgaccagggcgcccgggtgctgcgcggcaccgaggtcaccggcctcgcccaggacgccgacgcggtcaccgtcaccgcccgcgccgacggcggcggacccgcttccacgtggcgagcccggtacgtcgtgggggcggacggggcgcacagcaccgtccgcggcctcctcggcgccgacttccccggaaggacggttctgacctccgtggtgctggccgatgtccgcctcgccgacggccccaccgggaacgggctcaccctgggcaacacccctgaggtcttcggcttcctcgtgccgtacgggaaggcgcgccccggctggtaccggtcgatgacctgggaccgccgccaccaactgcccgacaaggccgccgtggaggaggcggaggtcacccgcgtactggccgaggccatgggacgtgacgtcggggtccgtgagatcggctggcactcccggttccactgcgatgaacgccaggtccgctcctaccggcacggccgggtcttcctcgccggggacgccgcccacgtgcactccccgatgggcggccagggcatgaacaccggcgtccaggacgcggccaacctcgcctggaagctcgacctcgccctcggcggcgccgacccggccatcctggacacctaccaccgggagcgccaccccgtcggccgccgtgtcctgctccagagcggtgccatgatgcgcgccgtcaccctcgggccgcgcccggcgcggtggctgcgcgaccatctggccccggccctgctgggcgtcggccgggtgcgcgacaccatcgccggaagcttcaccggcgtcaccccgcgctatccgcgcggacggcgacagcacgcactggtgggcacccgcgccaccgaagtcccgctcgccgagggccggttgaccgaactgcagcgggccggtggctttctgctgatccgcgagcggggcgcggcgcgcgtcgacaccacggtggcccaggccgagcgcaccgactccggccccgccctgctggtccgccccgacggctatatcgcctgggccggacccggtgtccgtacggacggccccgacggctggcacaccacatggcgggcctggaccggcccggccaccgatgcggtgcgcgccgggcgctgaacaggagacggggagacggcgccgggcgggcggcccggcgccgacgccctcatccgttccccgtcgcctgcccggcggacagggagtcggggagggcagcggcggccggttcctcgggtcgtcccttgcggaagaaccggagcagcgacgggccgccccggaaacaccacagcgcgatgaccggatcgcagatcgcacagcccagcgacgagccatgcgcgaacgggacgatggggttgcccttgatgtgatgcacgatgtcccacgcggtgtgcagcagccagccgatgccgatgaaggtccacgactccaggccacggtaggccacataggtggcgaccacggtgaaggcgaactcccagccgtccaggccgccgccgctgaggtaggccgcacccgctccgccgaccatgatcgcgttgaagcgccggcggtgcggttcgcgaatcagggacatcaggagcgcgtagaggacaccgatgaagaccggagcgatgtattggatcatgcggaagaacttcctgcgggtgacggaacgttggccgcccggcggggcgacggttcatcacgctagatccgcccccggccgccccacagggccattcccgacacgctccaacggataatcgccggggccggatcatcgccgtggccacggcctccacccggccaccacgctcagggcccgatcacagcagccgccacaggtggtcatcggttccgttgtcgtcgtactgcaccacctgggcgctgttggcggtggacatgccgtcgacacccagcaccttctggctgttcttgttgaggacgcggaaccagccgtcgccgttgtccaccttccgccagaggtgatcggccgtgccgttgtcctcgtactggacgacgatggcgctgttcgcggtggacatccggtcgacgcccaggaccttgccgctgtggccgttgcggatcaggaaccagccgtcgccccggtcgatccactgccaggcgtggtcgcccgtcggtgtgttgtcgtactgcaccacgcgggcgctgttggccgtggacatctcgtcgacggcgagcaccttgccgctgtgcttgttgagcagtcggcggaagggcggctccggggtccacgcccggccgtccggcgcggccgtgggaaagcaggtgatacgcagccgggcggcgcccatggggatgagggtgaccgtctccgccggtgcgtcggcccgggccgggctctgctgaagcggggtgaccacatgctcgtcgtccgagacccactcggcgatacggcgcgcctgggcggtcatgcggaccggggtggtctcgtgggtgaagggattggcggcgagcggaccgtcgtcgcgggtgagcacggggagggctccgggggcgaggccgtagttccacggagtggtggcgtgcacttcgtactcggggaaggtgtcggtgccggcgtagcgcacgaagtcctcgccgatgcgcagggagtacgtcagcgggccgtggtcgacgctgaccgcgccgtgctgcgccgaccaggtccgcagggcggtgcgctgcggcaggcggatcgtcaccacatcgccgtccgtccagctccggtcgaccttgacgaaggccggaccgccgcgcgtggccaccgcccggccgttgacctcgatccgggggttcttgcaccagccggggacccgcagatggagcgggaaggccaccttctcgggggtggacagcgtgagtgtgatggtctcgtcgaacggatagtcggtgtcctcggtgacggtgaccgtcgtaccgcccgccaccttcgcggacacctggcttgcggcgtacagggaggcggcgagccccttgtcgggcgtggccagccacagctcctcgctgaagtacggccagcccatgccgtagttgtgcggacagcagcggtactggtcgacgcccggctggtacgactgcatcgcgaagccgttctggaactgcccctgcgacttcaccgcgttgttcagatcgatgctgttcgcgctggtgatgtagtgggtgccggtgccctgggggtcgagggcggcgggcagcatgttgaacgccaggtcctcgcaccggtcggcccacaccggatcgccggtgatccgggtcagcagctcatggctggccatgaattcgacgatgccgcaggtctcgaagccctgccgggggtctccgaaacccgggcggtagttctcgtccccggcgaagccaccgcccgggaactggccgtatgcgccgagcaccgacgtatagccgcggtaggtcgcctgcctgagctcggcggagccggtcagctgggcgtactgggcgggctcgcggaagccctgggcgatattgacgttgtgcggggtcgggatgttgtcgacccaatt

(19) United States
(12) Patent Application Publication
(10) Pub. No.: US 2009/0075283 A1
(43) Pub. Date: Mar. 19, 2009

(54) Vectors and Methods for Cloning Gene Clusters or Portions Thereof

(75) Inventors: Hongbo Liu, Peabody, MA (US); Min He, Congers, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/179,782

(22) Filed: Jul. 25, 2008

Related U.S. Application Data
(60) Provisional application No. 60/962,311, filed on Jul. 27, 2007.

(51) Int. Cl. C12Q 1/68; C12N 15/00; C12P 21/04

(52) U.S. Cl. 435/6; 435/320.1; 435/69.1

(57) ABSTRACT

The present invention relates to a shuttle BAC vector for facilitating the cloning, transfer and heterologous expression of streptomycete secondary metabolite biosynthetic gene clusters. The invention also relates to a plasmid rescue method using this vector for enhancing the process of cloning biosynthetic gene clusters for secondary metabolites from streptomycetes without sophisticated generation and screening of cosmids or BAC libraries. The cloned DNA can then be used for sequencing or heterologous expression of putative secondary metabolic gene clusters.

### CROSS-REFERENCE TO RELATED APPLICATIONS

This application claims the benefit under 35 U.S.C. §119 (e) of U.S. Provisional Application Ser. No. 60/962,311, filed Jul. 27, 2007, the disclosure of which is incorporated by reference herein in its entirety.

### FIELD OF THE INVENTION

The present invention relates to vectors and methods for cloning or transferring large nucleic acid fragments containing whole or portions of a gene cluster from one prokaryotic organism to another. The invention also relates to a plasmid rescue method for isolating chromosomal DNA adjacent to an inserted piece of DNA in various organisms.

### BACKGROUND OF THE INVENTION

The gram-positive bacteria in the order Actinomycetales, including Actinomycetes and Streptomycetes, produce enormous amounts of natural products with useful pharmaceutical activities. Many of these natural products belong to either polyketide or nonribosomal peptide families that have a very diversified chemical structure and bioactivities. Polyketide synthases (PKSs) and nonribosomal peptide synthestases (NRPSs), are multimodular meganzymes, which are responsible for the synthesis of the corresponding chemicals (Staunton J, Weissman K. J. 2004. Nat Prod Rep 2001, 18:380-416; Finking R, Marahiel M A, Annu Rev Microbiol, 58:453-488; Annu Rev Microbiol. 2004; 58:453-88.). The genes that encode the multienzyme systems are usually grouped together on the chromosome and form distinct biosynthetic gene clusters. Depending on the final product, the gene clusters can be as large as 100 kb in size (August, P. R., Tang, L., Yoon, Y. J., Ning, S., Müller, R., Yu, T. W., Taylor, M., Hoffmann, D., Kim, C. G., Zhang, X., Hutchinso, C. R., and Floss, H. G. 1998. S699. Chem. Biol. 5:69-79.). This phenomenon presents a challenge for cloning of the large gene cluster in one clone, which is especially useful for heterologous expression studies. Although the application of bacterial artificial chromosomes (BAC) facilitates the cloning process, the transferring of any large gene cluster from E. coli to Streptomycetes still remains challenging. Recently, E. coli-Streptomyces shuttle BAC vectors have been built to overcome this challenge (Sosio, M., F. Giusino, C. Cappellano, E. Bossi, A. M. Puglia, and S. Donadio. 2000. Nat. Biotechnolo. 18:343-345; Martinez, A. et al., 2004, Appl. Environ. Microbiol., 70(4):2452-63). In both studies, the φC31 attP-int recombination system was utilized to integrate the vector site-specifically into the chromosome.

The conventional method used for cloning large gene clusters from Streptomycetes usually involves the complicated and laborious construction and screening of either cosmid or BAC libraries. Whole genome sequencing data is now available for numerous Streptomycetes strains. Additionally, increased characterization of natural product biosynthetic pathways is an increasing area of interest.

A plasmid rescue method has been used to isolate the chromosomal DNA adjacent to an inserted piece of DNA in various organisms (Hamilton, B. A., Zinn, K. 1994. Methods Cell Biol. 44:81-94; Kiessling, U., Platzer, M., and Strauss, M. 1984. Mol Gen Genet. 193:512-519; Weinrauch, Y., and Dubnau, D. 1983. J Bact. 154:1077-1087; McMahon T. L., Wilczynska, Z., Barth, C., Fraser, B. D., Pontes, L., and Fisher P. R. 1996. Nucleic Acids, Res. 24:4096-4097). Due to the limited cloning capacity of the vectors used for this purpose, this method has been used primarily for cloning and identification of regions immediately adjacent to the site of insertion.

The citation of any reference herein is not an admission that such reference is available as prior art to the instant invention.

### SUMMARY OF THE INVENTION

The invention provides a shuttle BAC vector for direct cloning of gene clusters ranging in size from about 20 kb to about 100 kb. The vector is used for the transfer and integration of cloned DNA from a prokaryotic organism into a strain of Actinomycetes. Integration of the cloned DNA occurs at the φBT1 attB site of the recipient chromosome. The invention also provides a plasmid (BAC) rescue method that can be used for cloning large DNA fragments directly from the designated Actinomycetes strain without the need for generation and screening of cosmid or BAC libraries. These large DNA fragments may contain intact gene clusters or a major portion of a gene cluster.

In a first aspect, the invention provides a vector for cloning or transfer of a large DNA fragment comprising a whole, or a portion of a gene cluster, from one prokaryotic organism to a species of Actinomycetes, comprising at least two origins of replication, a prokaryotic F factor partitioning system, an origin of transfer, a site-specific recombination system that allows for the integration of the vector into the recipient cell and a selection marker.

In a second aspect, the invention provides a vector for cloning or transfer of a large DNA fragment comprising a whole, or a portion of a gene cluster, from one prokaryotic organism to a species of Actinomycetes, comprising at least two origins of replication, a prokaryotic F factor partitioning system, an origin of transfer, a φBT1 attP-int recombination system and a selection marker.

In one embodiment, the invention provides a vector as described herein that further comprises a whole or a portion of a gene cluster.

In one embodiment, the prokaryotic organism from which the vectors of the present invention are transferred is E. coli. In other embodiments, the prokaryotic organism may be the same or a different strain of actinomycetes, or any other prokaryotic organism known to those skilled in the art for transfer of genetic material from one organism to another. The donor of the genetic material may be a prokaryotic or eukaryotic organism.

In one embodiment, the vector of the present invention is a Bacterial Artificial Chromosome (BAC) vector. In one embodiment, the BAC vector is a shuttle BAC vector. In one embodiment, the shuttle BAC vector is an E. coli-Actinomycetes conjugative vector, pSBAC (SEQ ID NO: 1).

In one embodiment, at least two origins of replication of a vector of the invention are E. coli origins of replication. In one embodiment, the two E. coli origins of replication are ori 2 and ori V. In one embodiment, at least one of the origins of replication is selected from ori 2 and ori V. In one embodiment, at least one of the origins of replication comprises the nucleotide sequence as set forth in SEQ ID NO: 2 or SEQ ID NO: 3. In one embodiment, the ori 2 nucleic acid sequence is set forth in SEQ ID NO: 2. In one embodiment, the ori V nucleic acid sequence is set forth in SEQ ID NO: 3.

In one embodiment, the prokaryotic F factor partitioning system of the vectors of the present invention is an E. coli F factor partitioning system. In one embodiment, the E. coli F factor partitioning system nucleic acid sequence comprises the nucleotide sequence as set forth in SEQ ID NO: 4.

In one embodiment, the origin of transfer of the vectors of the present invention is oriT. In one embodiment, the origin of transfer comprises the nucleotide sequence as set forth in SEQ ID NO: 5.

In one embodiment, the shuttle BAC vector comprises the nucleotide sequence of SEQ ID NO: 1.

In one embodiment, a vector of the present invention provides for cloning or transfer of a large DNA fragment comprising a whole, or a portion of a gene cluster that encodes one or more gene product(s) that are part of a specific biosynthetic pathway for secondary metabolites. In one embodiment, the gene product(s) is selected from a polyketide and a non-ribosomal polypeptide (NRP). In one embodiment, the polyketide is selected from the group consisting of an antibiotic, an immunosuppressant, an anti-cancer agent, an anti-fungal agent and a cholesterol lowering agent. In one embodiment, the polyketide of the invention is a macrolide antibiotic or a tetracycline antibiotic. In one embodiment, the macrolide antibiotic is selected from the group consisting of azithromycin, clarithromycin, dirithromycin, erythromycin and troleandomycin. In one embodiment, the tetracycline is selected from the group consisting of chlortetracycline, oxytetracycline, and demeclocycline. In one embodiment, the polyketide of the invention is an immunosuppressant selected from the group consisting of rapamycin, ascomycin (FK520) and tacrolimus (FK-506). In one embodiment, the polyketide of the invention is the anti-cancer agent doxorubicin. In one embodiment, the polyketide of the invention is the anti-fungal agent amphotericin B. In one embodiment, the polyketide of the invention is the cholesterol lowering agent lovastatin. In one embodiment, the non-ribosomal polypeptide (NRP) of the invention is an immunosuppressant or an antibiotic. In one embodiment, the non-ribosomal polypeptide (NRP) of the invention is the immunosuppressant cyclosporine A. In one embodiment, the non-ribosomal polypeptide (NRP) of the invention is the antibiotic penicillin. In one embodiment, a vector of the present invention provides for cloning or transfer of a large DNA fragment comprising a whole, or a portion of a gene cluster that encodes the proteins that are involved in the biosynthesis of actinorhodin or meridamycin. In one embodiment, a vector of the present invention provides for cloning or transfer of a large DNA fragment comprising a whole, or a portion of a gene cluster that encodes the proteins that are involved in the biosynthesis of meridamycin, wherein the gene cluster is the mer gene cluster, which comprises the nucleic acid sequence of SEQ ID NO: 31.

A third aspect of the invention provides a plasmid rescue method for isolating or cloning a large DNA fragment, wherein the large DNA fragment ranges in size from about 20 kb to about 100 kb, the method comprising transferring any of the vectors of the present invention to a recipient Actinomycetes cell, which contains a nucleic acid having a site specific integration sequence that allows for the integration of the vector, selecting for the recipient Actinomycetes cell that contains the vector incorporated into the Actinomycetes chromosome, isolating the DNA from the chromosome of the recipient Actinomycetes cell, transferring the DNA into an E. coli cell, screening for an E. coli cell that contains any of the vectors and isolating the large DNA fragment from the E. coli cell.

A fourth aspect of the invention provides a plasmid rescue method for isolating or cloning a large DNA fragment, wherein the large DNA fragment ranges in size from about 20 kb to about 100 kb, the method comprising transferring any of the vectors of the present invention to a recipient Actinomycetes cell, which contains a homologous sequence that allows for the integration of the vector, selecting for the recipient Actinomycetes cell that contains the vector incorporated into the Actinomycetes chromosome, isolating the DNA from the chromosome of the recipient Actinomycetes cell, transferring the isolated DNA from the previous step into an E. coli cell, screening for an E. coli cell that contains any of the vectors of the invention and isolating the large DNA fragment from the E. coli cell.

In one embodiment, the plasmid rescue method(s) of the invention provide for isolating or cloning a large DNA fragment, wherein the large DNA fragment is a whole or a portion of a gene cluster. In one embodiment, the gene cluster encodes the proteins that are involved in the biosynthesis of actinorhodin or meridamycin.

In one embodiment, the plasmid rescue method of the invention provides a site specific integration sequence in the recipient cell, which is an aft site. In one embodiment, the att site in the recipient cell is an attB site comprising the nucleotide sequence of SEQ ID NO: 6.

In one embodiment, the plasmid rescue method of the invention provides a selecting step, which comprises selecting for a biological or enzymatic activity that is transferred to the recipient cell by the vector.

In one embodiment, the plasmid rescue method of the invention provides that the transferring of the vector comprises conjugating the donor cell containing the vector with a recipient Actinomycetes cell.

A fifth aspect of the invention provides a method of producing meridamycin comprising expressing the amino acids encoded by the mer gene cluster of SEQ ID NO: 31.

In one embodiment, the mer gene cluster is incorporated into the pSBAC vector of SEQ ID NO: 1.

These and other aspects of the present invention will be better appreciated by reference to the following drawings and Detailed Description.

### BRIEF DESCRIPTION OF THE DRAWINGS

FIG. 1. Map of the E. coli-Streptomycetes BAC Vector pSBAC

FIG. 2. Schematic Representation of Plasmid Rescue and analysis of the Rescued Clones

FIG. 3. Schematic Representation of the Strategy used to Rescue the act Gene Cluster from S. coelicolor and analysis of the Rescued Clones

FIG. 4. Absorption Spectra of Crude Extracts of S. coelicolor, S. lividans K4-114 and Complementation Strain ACTres12

FIG. 5. (A) Cloning of the whole mer gene cluster into pSBAC vector. Schematic representation of the mer gene cluster is shown. The arrow represents the translational start codon site for MerP gene. The solid line represents the probe used for library screening, and the hatched line represents the probe used for Southern hybridization (B) Southern analysis confirmed the introduction of the mer gene cluster into the heterologous hosts.

FIG. 6. Semi-quantitative RT-PCR analysis of the transcription of mer gene cluster in various strains.

FIG. 7. LC/MS analysis of fermentation extracts of S. lividans K4-114, HL30-K3, E7, original meridamycin producer NRRL 30748 and the meridamycin and 3-Normerdiamycin standard.

FIG. 8. HRMS confirmation of the production of merdamycin and 3-Normeridamycin from train E7 grown in FKA medium supplemented with 4% proline and 10 mM diethymalonate.

### DETAILED DESCRIPTION

Before the present methods and treatment methodology are described, it is to be understood that this invention is not limited to particular methods, and experimental conditions described, as such methods and conditions may vary. It is also to be understood that the terminology used herein is for purposes of describing particular embodiments only, and is not intended to be limiting.

As used in this specification and the appended claims, the singular forms “a”, “an”, and “the” include plural references unless the context clearly dictates otherwise. Thus, for example, references to “the method” includes one or more methods, and/or steps of the type described herein and/or which will become apparent to those persons skilled in the art upon reading this disclosure and so forth.

Accordingly, in the present application, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Although any methods and materials similar or equivalent to those described herein can be used in the practice or testing of the invention, the preferred methods and materials are now described. All publications mentioned herein are incorporated by reference in their entirety.

### DEFINITIONS

The terms used herein have the meanings recognized and known to those of skill in the art, however, for convenience and completeness, particular terms and their meanings are set forth below.

The term “about” means within 20%, preferably within 10%, and more preferably within 5%. In one embodiment of the present invention, the term “about” refers to the size of the gene clusters as described in the present invention, which range from 20 kb to 100 kb. In one embodiment, the term “about” refers to the actual sizes or ranges as described herein.

“Actinomycetes” are non-motile, filamentous, gram positive bacteria. As Actinomycetes grow, they form branching filaments of cells which become a network of strands called a mycelium, similar in appearance to the mycelium of some fungi. Actinomycetes are also unique in the way they form spores and in the production of numerous antibiotics. By far the most successful genus in this group is Streptomyces with over 500 species. The Streptomycetes are members of the bacterial order Actinomycetales, bacteria that resemble fungi in their branching filamentous structure. However, they are true bacteria—prokaryotic cells—unlike eukaryotic fungal cells. Streptomyces species refers to a terrestrial actinomycete, which produces macrolide antibiotic complexes.

Actinorhodin refers to a blue-pigmented aromatic polyketide antibiotic from Streptomyces coelicolor, whose basic carbon skeleton is derived from type II polyketide synthase (PKS). (A. Zeeck and P. Christiansen. Liebigs Ann. Chem. 724 (1969), pp. 172-182).

An “antibiotic biosynthetic pathway” includes the entire set of antibiotic biosynthetic genes necessary for the process of converting primary metabolites into antibiotics. These genes can be isolated by methods well known to the art, e.g., see U.S. Pat. No. 4,935,340.

An “att” site refers to a site having nucleic acid identity or similarity that facilitates site-specific recombination between two nucleic acid molecules. For example, one att site described in the present invention is the integration site for φBT1 bacteriophage (Gregory, M A, et al., 2003, J. Bacteriol. 185, No. 17: 5320-5323). The “attB” site refers to the attachment site on the bacterial cell chromosome, whereas the “attP” site refers to the attachment site on the bacteriophage. The nucleic acid sequence for the attP site in the bacteriophage for the φBT1 system is shown in SEQ ID NO: 7, whereas the nucleic acid sequence for the attB site in the bacterial cell (Actinomycetes) for the φBT1 system is shown in SEQ ID NO: 6. Another example of an “att” site is the φC31 att site described by Bierman et al. (Bierman, M., R. Logan, K. O'Brien, E. T. Seno, R. N. Rao, and B. E. Schoner. (1992). Gene 116:43-49 and Kieser, T., M. J. Bibb, M. J. Buttner, K. F. Chater, and D. A. Hopwood. (2000). Practical Streptomyces genetics. University of Nottingham, Nottingham, UK).

As used herein, the term “BAC” (Bacterial Artificial Chromosome) is intended to mean a cloning and sequencing vector derived from a bacterial chromosome into which a large DNA fragment can be inserted. The large DNA fragment may range in size from about 20 kb to about 400 kb. In one embodiment, the large DNA fragment may range in size from about 20 kb to about 300 kb. In one embodiment, the large DNA fragment comprises a whole or a portion of a gene cluster ranging in size from about 20 kb to about 100 kb. The large DNA fragment BACs are based on the single-copy F-plasmid of E. coli and have been demonstrated previously to stably maintain human genomic DNA of >300 kb, and genomes of large DNA viruses, including those of baculovirus and murine cytomegalovirus (Shizuya, H., et al., Proc. Natl. Acad. Sci. USA 89:8794 8797 (1992); Luckow, V. A., et al., J. Virol. 67:4566 4579 (1993); Messerle, M., et al., Proc. Natl. Acad. Sci. USA 94:14759 14763 (1997)). As used herein, the term “Recombinant Bacterial Artificial Chromosome” (BAC) refers to a BAC vector containing a large DNA insert, ranging in size from about 20 kb up to about 400 kb in size. In one embodiment, the large DNA insert comprises a whole or a portion of a gene cluster of about 20 kb to about 100 kb, encoding one or more gene product(s) that are part of a specific biosynthetic metabolic pathway. Once the Recombinant BAC DNA has been introduced into a host bacterium, it can either replicate autonomously or integrate into the host chromosome.

The term “biosynthetic pathway for secondary metabolites” refers to a biosynthetic network composed of genes from bacteria, humans, and various plants for synthesizing secondary metabolites for pharmaceutical use. The pathway generally involves a series of naturally occurring enzyme controlled reactions whereby one substance is converted to another, resulting in the release of secondary metabolites or by-products.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, the term “conjugation” refers to the direct transfer of nucleic acid from one prokaryotic cell to another via direct contact of cells. Thus, a “conjugative vector”, (for example, pSBAC) is a vector that contains a nucleic acid of interest, whereby such nucleic acid is directly transferred (ie. the passing of a nucleic acid sequence from one cell to another without isolation of the sequence) from one cell to another via direct contact between the cell containing the vector and a recipient cell to which the nucleic acid is transferred following direct contact of the two cells. As used herein, the term “conjugative transfer” refers to the temporary union of two bacterial cells during which one cell transfers part or all of its genetic material to the other.

The term “derivative” refers to a chemically synthesized organic molecule that is functionally equivalent to the active parent compound, but may be structurally different. It may also refer to chemically similar compounds, which have been chemically altered to increase bioavailability, absorption, or to decrease toxicity. For example, a derivative is a compound that is formed from a similar compound or a compound that can be expected to arise from another compound, if one atom is replaced with another atom or group of atoms, or a compound that may be formed from a precursor compound.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “expression control sequence” refers to a promoter and any enhancer or suppression elements that combine to regulate the transcription of a coding sequence. In a preferred embodiment, the element is an origin of replication.

“F factor” or “prokaryotic F factor” refers to a fertility factor found in prokaryotes. It is a small piece of episomal DNA that enables bacteria to mediate conjugation with other bacteria. In its extrachromosomal state the factor has a molecular weight of approximately 62 kb and encodes at least 20 transfer genes, an origin of replication as well as other genes for incompatibility and replication. The F factor can exist in three different states: “F+” refers to a factor in an autonomous, extrachromosomal state containing only the genetic information described above. The “Hfr” (which refers to “high frequency recombination”) state describes the situation when the factor has integrated itself into the chromosome presumably due to its various insertion sequences. Finally, the “F′” or (F prime) state refers to the factor when it exists as an extrachromosomal element, but with the additional requirement that it contain some section of chromosomal DNA covalently attached to it. A strain containing no F factor is said to be “F⁻”. The F factor “partitioning system” refers to the system that ensures both daughter cells inherit a copy of the parental plasmid.

“Fragment” refers to either a protein or polypeptide comprising an amino acid sequence of at least 4 amino acid residues (preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the amino acid sequence of a parent protein or polypeptide, or a nucleic acid comprising a nucleotide sequence of at least 10 base pairs (preferably at least 20 base pairs, at least 30 base pairs, at least 40 base pairs, at least 50 base pairs, at least 50 base pairs, at least 100 base pairs, at least 200 base pairs) of the nucleotide sequence of the parent nucleic acid. Any given fragment may or may not possess a functional activity of the parent nucleic acid or protein or polypeptide.

The term “gene”, means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription.

The term “gene cluster” refers to any group of two or more closely linked genes that encode for the same or similar products. In the present invention, the gene clusters encode the multimodular meganzymes (multienzyme systems) responsible for the synthesis of secondary metabolites, as defined herein. For example, the polyketide synthases (PKSs) and the non-ribosomal peptide synthetases (NRPSs) are both multimodular meganzymes responsible for synthesis of the corresponding chemicals (See Staunton J. et al. Nat Prod Rep. (2001) August; 18(4):380-416; Finking, R. et al. Annu Rev Microbiol. 2004; 58:453-88). The genes that encode these multienzyme systems are usually grouped together on the chromosome and form distinct biosynthetic gene clusters.

“Gene Product” as used herein, refers to a product produced by a gene when that gene is expressed. Typically, the phrase refers to a nucleic acid, a protein or a polypeptide. For example, in the present invention the phrase refers to an enzyme such as a polyketide synthase, or any enzyme that plays a role in the synthesis of a non-ribosomal polypeptide, or it may refer to the actual polyketide or non-ribosomal polypeptide as well. Examples of this may be found in U.S. patent publications 20050272133, or 20030134398 and 20030124689. Further examples may be found in U.S. Pat. No. 6,495,348.

The term “heterologous” refers to a combination of elements not naturally occurring. For example, heterologous DNA refers to DNA not naturally located in the cell, or in a chromosomal site of the cell. The heterologous DNA may include a gene foreign to the cell. A heterologous expression regulatory element is an element operatively associated with a different gene than the one it is operatively associated within nature.

“Homologous recombination” is a type of genetic recombination, a process of physical rearrangement occurring between two different strands of DNA molecules. Homologous recombination involves the alignment of identical or similar sequences, a crossover between the aligned homologous DNA strands of the two molecules, and breaking and repair of the DNA to produce an exchange of material between the strands. Homologous recombination is distinguished from other types of recombination. For example, “site specific recombination”, as exemplified by invertible elements, resolvases, and some phage integration events are examples of non-homologous recombination. Though in many cases identical or similar sequences are required at the two recombining sites, the sequences are short, distinguishing them from the longer stretches (hundreds of base pairs) used in homologous recombination. (J Rubnitz and S Subramani. 1984, Mol Cell Biol. 4: 2253-2258).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., supra). The conditions of temperature and ionic strength determine the “stringency” of the hybridization. For preliminary screening for homologous nucleic acids, low stringency hybridization conditions, corresponding to a Tₘ (melting temperature) of 55° C., can be used, e.g., 5×SSC, 0.1% SDS, 5×Denhardt's, and no formamide; or 30% formamide, 5×SSC, 0.5% SDS, 5×Denhardt's). Moderate stringency hybridization conditions correspond to a higher Tₘ, e.g., 40% formamide, with 5× or 6×SCC, 5×Denhardt's. High stringency hybridization conditions correspond to the highest Tₘ, e.g., 50% formamide, 5× or 6×SCC, 5×Denhardt's. SCC is a 0.15M NaCl, 0.015M Na-citrate buffer. 5×Denhardt's is 0.1% ficoll, 0.1% polyvinylpyrrolidone, 0.1% g BSA (w/v). Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived (see Sambrook et al., supra, 9.50-9.51). For hybridization with shorter nucleic acids, i.e., oligonucleotides, the position of mismatches becomes more important, and the length of the oligonucleotide determines its specificity (see Sambrook et al., supra, 11.7-11.8). A minimum length for a hybridizable nucleic acid is at least about 10 nucleotides; preferably at least about 15 nucleotides; and more preferably the length is at least about 20 nucleotides.

The term “integration site” refers to the site of insertion of a nucleic acid into the genome of a recipient cell. The site of integration may be random, or it may occur via a site-directed mechanism, known to those skilled in the art. In conservative site-specific recombination, for example, a mobile DNA element is inserted into a strand of DNA by means similar to that seen in crossover. A segment of DNA on the mobile element matches exactly with a segment of DNA on the target, allowing enzymes called integrases to insert the rest of the mobile element into the target. Integrases are a special type of recombinase enzyme. Recombinases are enzymes which cleave the double stranded DNA at specific sites resulting in a loss of the phosphodiester bonds. This reaction is stabilized by the formation of a covalent bond between the recombinase and the DNA through a phospho tyrosine bond. Another form of site-specific recombination, transpositional recombination does not require an identical strand of DNA in the mobile element to match with the target DNA. Instead, the integrases involved introduce nicks in both the mobile element and the target DNA, allowing the mobile DNA to enter the sequence. The nicks are then removed by a ligase. Recombination between DNA sequences that contain no sequence homology, is also referred to as non-homologous end joining.

In general terms, the word “isolating” refers to the removal of a material of interest from its original environment (e.g., a natural environment if it is naturally occurring, or from an environment into which it has been placed). For example, an “isolated” peptide or protein is substantially free of cellular material or other contaminating proteins from the cell or tissue source from which the protein is derived, or substantially free of chemical precursors or other chemicals when chemically synthesized. In the present invention, the plasmid rescue method provides for a means of “isolating” or recovering a gene cluster that lies adjacent to the integration site, such that it is free of any other contaminating genetic or cellular material.

As used herein, the term “large DNA fragment” refers to a piece of DNA that has an approximate size ranging from about 20 kilobases to about 400 kilobases. In one embodiment, the “large DNA fragment” may range from about 20 kb to about 300 kb. In one embodiment, the “large DNA fragment” may range from about 20 kb to about 200 kb. In one embodiment, the “large DNA fragment” may range from about 20 kb to about 100 kb. In one embodiment, the “large DNA fragment” may comprise a whole or a portion of a gene cluster.

“Meridamycin” is a macrolide polyketide that has been shown to have strong FKBP12 binding activity and significant neuroprotective activity in vitro, having the structure (I):

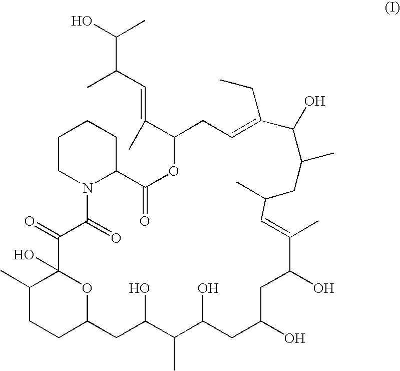

It is produced by terrestrial actinomycetes Wyeth culture LL-BB0005, deposited under the terms of the Budapest Treaty with the Agricultural Research Service Culture Collection (NRRL) on May 18, 2004 (Accession No. NRLL 30748). Meridamycin functions as an immunophilin ligand which binds to FK-binding proteins.

A “natural product” is a chemical compound or substance produced by a living organism, which is found in nature and which usually has a pharmacological or biological activity for use in pharmaceutical drug discovery and design. A natural product can be considered as such even if it can be prepared by total synthesis. Not all natural products can be fully synthesized and many natural products have very complex structures, some of which are too difficult and expensive to synthesize on an industrial scale. Such compounds can only be harvested from their natural source. Furthermore, the number of structural analogues that can be obtained from harvesting is severely limited. Semisynthetic procedures are sometimes used to get around these problems. This often involves harvesting a biosynthetic intermediate from the natural source, rather than the final (lead) compound itself. The intermediate could then be converted to the final product by conventional synthesis. This approach can have two advantages. First, the intermediate may be more easily extracted in higher yield than the final product itself. Second, it may allow the possibility of synthesizing analogues of the final product. The semisynthetic penicillins are an illustration of this approach. Another example is that of paclitaxel. It is manufactured by extracting 10-deacetylbaccatin III from the needles of the yew tree, then carrying out a four-stage synthesis.

The term “non-ribosomal polypeptides” refers to polypeptides that are synthesized using a modular enzyme complex, which functions much like a conveyor belt. Nonribosomal peptides are confined primarily to unicellular organisms, plants and fungi. All of these complexes are laid out in a similar fashion, and they can contain many different modules to perform a diverse set of chemical manipulations on the developing product. In general, these peptides are cyclic (often with highly-complex cyclic structures), although linear nonribosomal peptides are common. Since the system is modular and closely related to the machinery for building fatty acids and polyketides, hybrid compounds are often found. Oxazoles, thiazoles and their reduced counterparts often indicate that the compound was synthesized in this fashion. Other examples of non-ribosomal polypeptides include: vancomycin, thiostrepton, ramoplanin, teicoplanin, gramicidin, and bacitracin.

“Polyketides” are secondary metabolites from bacteria, fungi, plants and animals. Secondary metabolites seem to be unnecessary for an organism's ontogeny, but appear to have applications such as defense and intercellular communication. Polyketides represent a large group of natural products that are derived from successive condensations of simple carboxylates, such as acetate, propionate or butyrate. They also serve as building blocks for a broad range of natural products or are derivatized. Polyketides are structurally a very diverse family of natural products with an extremely broad range of biological activities and pharmacological properties. Polyketide antibiotics, antifungals, cytostatics, anticholesterolemics, antiparasitics, coccidiostatics, animal growth promotants and natural insecticides are in commercial use. Examples include: Macrolides, such as picromycin, erythromycin A, clarithromycin, and azithromycin. Also included are the immunosuppressants tacrolimus (FK506) and rapamycin, and the polyene antibiotics, e.g. amphotericin. Also included are the tetracycline family of antibiotics. The anti-cancer compounds, daunomycin, bryostatin and discodermolide, are also polyketides. The veterinary compounds monensin and avermectin are also polyketides. Also Included are actinorhodin and meridamycin. Polyketides are synthesized by one or more specialized polyketide synthase (PKS) enzymes (See U.S. Patent Publication 20070148717).

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester analogs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear (e.g., restriction fragments) or circular DNA molecules, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA molecule” is a DNA molecule that has undergone a molecular biological manipulation.

A “nucleotide” refers to a subunit of DNA or RNA consisting of nitrogenous bases (adenine, guanine, cytosine and thymine), a phosphate molecule, and a sugar molecule (deoxyribose in DNA and ribose in RNA).

In order to propagate a vector in a host cell, it may contain one or more “origins of replication” sites (often termed “ori”), which is a specific nucleic acid sequence at which replication is initiated. Accordingly, the term “origin of replication” or “ori”, as used herein refers to a nucleic acid sequence that initiates nucleic acid replication. At the origin, the two strands of DNA are pulled apart to form a replication bubble. This creates a region of single stranded DNA on each side of the bubble. The DNA polymerase machinery can then move in and begin to synthesize the new strands of DNA, using the old strands as templates. A replication “fork” moves along the DNA in either direction from the origin, synthesizing new DNA.

“Ori 2” refers to an “origin of replication” from an E. coli plasmid that allows for single-copy replication. (Shizuya, H., Birren, B., Kim, U.-J., Mancino, v., Slepak, T I, Tachiiri, Y., and Simon, M. 1992. Proc. Natl. Aca. Sci. 890:8794-8797).

“Ori V” refers to an “origin of replication” from an E. coli plasmid that allows for high-copy replication. (Perri, S, and Helinski, D. R. 1993. DNA sequence requirements for interaction of the RK2 replication initiation protein with plasmid origin repeats. J. Biol. Chem. 268:3662-2669).

“Ori T” refers to an “origin of transfer” that permits the transfer of the vector from one bacterial cell to another.

The “origin of transfer” (e.g, oriT) represents the site on the vector where the transfer process is initiated. It is also defined genetically as the region required in cis to the DNA that is to be transferred. Conjugation-specific DNA replication is initiated within the oriT region which also encodes plasmid transfer factors.

“φBT1 attP-int recombination system” refers to a site-specific recombination system that permits site-specific integration of the vector into the attB site of the recipient cell's chromosome. (Gregory, M. A., Till, R., and Smith, M. C. M. 2003. J Bacteriol. 185:5320-5323; GenBank Accession Number AJ550940)

A “polynucleotide” or “nucleotide sequence” is a series of nucleotide bases in a nucleic acid, such as DNA and RNA, and means any chain of two or more nucleotides. A nucleotide sequence typically carries genetic information, including the information used by cellular machinery to make proteins and enzymes. These terms include double or single stranded genomic and cDNA, RNA, any synthetic and genetically manipulated polynucleotide, and both sense and anti-sense polynucleotide (although only sense stands are being represented herein). This includes single- and double-stranded molecules, i.e., DNA-DNA, DNA-RNA and RNA-RNA hybrids, as well as “protein nucleic acids” (PNA) formed by conjugating bases to an amino acid backbone. This also includes nucleic acids containing modified bases, for example thio-uracil, thio-guanine and fluoro-uracil. The nucleic acids herein may be flanked by natural regulatory (expression control) sequences, or may be associated with heterologous sequences, including promoters, internal ribosome entry sites (IRES) and other ribosome binding site sequences, enhancers, response elements, suppressors, signal sequences, polyadenylation sequences, introns, 5′- and 3′-non-coding regions, and the like. The nucleic acids may also be modified by many means known in the art. Non-limiting examples of such modifications include methylation, “caps”, substitution of one or more of the naturally occurring nucleotides with an analog, and internucleotide modifications such as, for example, those with uncharged linkages (e.g., methyl phosphonates, phosphotriesters, phosphoramidates, carbamates, etc.) and with charged linkages (e.g., phosphorothioates, phosphorodithioates, etc.). Polynucleotides may contain one or more additional covalently linked moieties, such as, for example, proteins (e.g., nucleases, toxins, antibodies, signal peptides, poly-L-lysine, etc.), intercalators (e.g., acridine, psoralen, etc.), chelators (e.g., metals, radioactive metals, iron, oxidative metals, etc.), and alkylators. The polynucleotides may be derivatized by formation of a methyl or ethyl phosphotriester or an alkyl phosphoramidate linkage. Furthermore, the polynucleotides herein may also be modified with a label capable of providing a detectable signal, either directly or indirectly. Exemplary labels include radioisotopes, fluorescent molecules, biotin, and the like.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase. The promoter may be operatively associated with other expression control sequences, including enhancer and repressor sequences.

“Prokaryotic F factor partitioning system” refers to an active positioning process that ensures proper segregation and faithful distribution of daughter F factors at cell division. (Niki, H. and Hiraga, S. 1997. Subcellular distribution of actively partitioning F plasmid during the cell division cycle in E. coli. Cell 90:951-957). This partitioning system contains three functionally distinct regions: two of them (sopA and sopB) encode gene products that act in trans, whereas the third region (sopC) functions in cis. All regions are essential in plasmid partitioning during cell division. (Ogura T., Hiraga S. 1983. Cell. 32:351-60).

The term “recipient cell” refers to a cell that is selected for receipt of the vector of interest, as described herein. The “recipient cell” may also be referred to as a “host cell”. For example, in the present invention, one embodiment provides for Actinomycetes as being a recipient cell. In one embodiment, the invention provides for a genus of Actinomycetes, in particular, a species of Streptomycetes, as being the recipient cell. In one embodiment, E. coli may be the recipient cell. Furthermore, a recipient cell may be one that is manipulated to express a particular gene, a DNA or RNA sequence, or a protein. Recipient cells can further be used for screening. Recipient cells may be cultured in vitro or one or more cells may be transferred to a non-human animal (e.g., a transgenic animal or a transiently transfected animal). The recipient cell may be selected from any biological organism, including prokaryotic (e.g., bacterial) cells, plant cells, and eukaryotic cells, including, insect cells, yeast cells and mammalian cells. Other representative examples of appropriate recipient cells include any other bacterial cell; fungal cells, such as yeast cells and Aspergillus cells; and insect cells such as Drosophila S2 and Spodoptera Sf9 cells.

“Secondary metabolites” are organic compounds that are not directly involved in the normal growth, development, or reproduction of organisms. Unlike primary metabolites, absence of secondary metabolites only results in mild impairment for the organisms such as: lowered survivability/fecundity, aesthetic differences, or else no change in phenotype at all. Secondary metabolites are often restricted to a narrow set of species within a phylogenetic group. The function or importance of these compounds to the organism is usually of an ecological nature as they are used as defenses against predators, parasites and diseases, for interspecies competition, and to facilitate the reproductive processes (coloring agents, attractive smells, etc). Since these compounds are usually restricted to a much more limited group of organisms, they have long been of prime importance in taxonomic research. Most of the secondary metabolites of interest to man fit into the following categories, and some fall into more than one. These categories are broad categories, which classify secondary metabolites based on their biosynthetic origin. Since secondary metabolites are often created by modified primary metabolite synthases, or “borrow” substrates of primary metabolite origin, these categories should not be interpreted as saying that all molecules in the category are secondary metabolites (for example the steroid category), but rather that there are secondary metabolites in these categories. Examples of these categories and samples within each category include: Alkaloids such as hyoscyamine, atropine, cocaine, codeine, morphine, tetrodotoxin; Terpenoids such as azadirachtin, artemisin, tetrahydrocannabinol; Steroids such as terpenes, Saponins; Glycosides such as Nojirimycin and glucosinolates; Phenols such as Resveratrol; Phenazines such as pyocyanin and phenazine-1-carboxylic acid (and derivatives).

The term “selecting” refers to the identification and isolation of a recipient cell that contains the vector of interest. Transformed microorganisms, that is, those containing recombinant molecules, may be selected with a variety of positive and/or negative selection methods or markers. In certain aspects, the positive selection marker is a gene that allows growth in the absence of an essential nutrient, such as an amino acid. For example, in the absence of thymine and thymidine, cells expressing the thyA gene survive, while cells not expressing this gene do not. A variety of suitable positive/negative selection pairs are available in the art. For example, various amino acid analogs known in the art could be used as a negative selection, while growth on minimal media (relative to the amino acid analog) could be used as a positive selection. Visually detectable markers are also suitable for use in the present invention, and may be positively and negatively selected and/or screened using technologies such as fluorescence activated cell sorting (FACS) or microfluidics. Examples of detectable markers include various enzymes, prosthetic groups, fluorescent markers, luminescent markers, bioluminescent markers, and the like. Examples of suitable fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of suitable bioluminescent markers include, but are not limited to, luciferase (e.g., bacterial, firefly, click beetle and the like), luciferin, aequorin and the like. Examples of suitable enzyme systems having visually detectable signals include, but are not limited to, galactosidases, glucorimidases, phosphatases, peroxidases, cholinesterases and the like. In other aspects, the positive selection marker is a gene that confers resistance to a compound, which would be lethal to the cell in the absence of the gene. For example, a cell expressing an antibiotic resistance gene would survive in the presence of an antibiotic, while a cell lacking the gene would not. For instance, the presence of a tetracycline resistance gene could be positively selected for in the presence of tetracycline, and negatively selected against in the presence of fusaric acid. Suitable antibiotic resistance genes include, but are not limited to, genes such as ampicillin-resistance gene, neomycin-resistance gene, blasticidin-resistance gene, hygromycin-resistance gene, puromycin-resistance gene, chloramphenicol-resistance gene, apramycin-resistance gene and the like. In certain aspects, the negative selection marker is a gene that is lethal to the target cell in the presence of a particular substrate. For example, the thyA gene is lethal in the presence of trimethoprim. Accordingly, cells that grow in the presence trimethoprim do not express the thyA gene. Negative selection markers include, but are not limited to, genes such as thyA, sacB, gnd, gapC, zwJ, talA, taiB, ppc, gdhA, pgi, Jbp, pyka, cit, acs, edd, icdA, groEL, secA and the like.

The term “selecting for a biological or enzymatic activity”, as used herein, refers to identifying and selecting the recipient cell, for example, the actinomycetes cell that contains the transferred vector by measuring for either the biological activity associated with the gene product that is transferred to the recipient cell by the vector, for example, an enzyme encoded by a gene cluster, such as, but not limited to, a polyketide synthase, or alternatively, measuring the activity of the enzyme itself. It may also refer to the biological activity of the final product, which may be a polyketide or a non-ribosomal polypeptide.

The term “selection marker” or “selectable marker” refers to the use of, or the inclusion of, a drug as a marker to aid in the cloning and identification of transformants, for example, genes that confer resistance to Apramycin, neomycin, puromycin, hygromycin, DHFR, GPT, zeocin and histidinol are useful selectable markers. Accordingly, cells containing a nucleic acid construct of the present invention may be identified in vitro or in vivo by including a marker in the vector. Such markers would confer an identifiable change to the cell permitting easy identification of cells containing the vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (“tk”) or chloramphenicol acetyltransferase (“CAT”) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

Accordingly, the term “sequence similarity” refers to the degree of identity or correspondence between nucleic acid or amino acid sequences of proteins that may or may not share a common evolutionary origin.

A “shuttle vector” refers generally to a plasmid that is capable of replicating in two different organisms, such as, for example, yeast and E. coli. In the present invention, the shuttle vector allows for transfer of large DNA fragments, including whole or portions of gene clusters, between E. coli and Actinomycetes. Moreover, the shuttle vector of the present invention is a “shuttle Bacterial Artificial Chromosome (BAC) vector”, which is a vector that allows for transfer of large fragments of DNA, from about 20 kb to about 400 kb, and which is capable of replicating in two different organisms.

A “site specific integration sequence” refers to a nucleic acid sequence in a donor or recipient nucleic acid molecule that facilitates recombination between the two nucleic acid molecules and integration of the donor nucleic acid molecule into the recipient nucleic acid molecule.

“Site-specific recombination” or “site-specific recombination system” refers to a recombination process between two DNA molecules that occurs at unique sites of each molecule which are generally 20-30 bases long, called attachment (att) sites. A specialized enzyme, the “integrase”, recognizes the two att sites, joins the two DNA molecules and catalyzes a DNA double-strand breakage and rejoining event that results in the integration of one of the DNA molecules into the other DNA of the recipient cell. (N. D. Grindley, K. L. Whiteson, P. A. Rice, 2006. Annu. Rev. Biochem. 75, 567-605.)

In a specific embodiment, the term “standard hybridization conditions” refers to a Tₘ of 55° C., and utilizes conditions as set forth above.

The language “substantially free of cellular material” includes preparations of a polypeptide/protein in which the polypeptide/protein is separated from cellular components of the cells from which it is isolated or recombinantly produced. Thus, a polypeptide/protein that is substantially free of cellular material includes preparations of the polypeptide/protein having less than about 30%, 20%, 10%, 5%, 2.5%, or 1%, (by dry weight) of contaminating protein. When the polypeptide/protein is recombinantly produced, it is also preferably substantially free of culture medium, i.e., culture medium represents less than about 20%, 10%, or 5% of the volume of the protein preparation. When polypeptide/protein is produced by chemical synthesis, it is preferably substantially free of chemical precursors or other chemicals, i.e., it is separated from chemical precursors or other chemicals which are involved in the synthesis of the protein. Accordingly, such preparations of the polypeptide/protein have less than about 30%, 20%, 10%, 5% (by dry weight) of chemical precursors or compounds other than polypeptide/protein fragment of interest. An “isolated” or “purified” nucleic acid molecule is one which is separated from other nucleic acid molecules which are present in the natural source of the nucleic acid molecule. Moreover, an “isolated” nucleic acid molecule, such as a cDNA molecule or an RNA molecule, or a gene cluster can be substantially free of other cellular material, or culture medium when produced by recombinant techniques, or substantially free of chemical precursors or other chemicals when chemically synthesized.

In a specific embodiment, two DNA sequences are “substantially homologous” or “substantially similar” when at least about 80%, and most preferably at least about 90 or 95% of the nucleotides match over the defined length of the DNA sequences, as determined by sequence comparison algorithms, such as BLAST, FASTA, DNA Strider, etc. An example of such a sequence is an allelic or species variant of the specific genes of the invention. Sequences that are substantially homologous can be identified by comparing the sequences using standard software available in sequence data banks, or in a Southern hybridization experiment under, for example, stringent conditions as defined for that particular system.

Similarly, in a particular embodiment, two amino acid sequences are “substantially homologous” or “substantially similar” when greater than 80% of the amino acids are identical, or greater than about 90% are similar. Preferably, the amino acids are functionally identical. Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA, etc.).

The term “transferring” refers to the introduction of a nucleic acid into a cell by any means including electroporation (making transient holes in cell membranes using electric shock), conjugation (refers to the direct transfer of nucleic acid from one prokaryotic cell to another via direct contact of cells), transduction (the process by which bacterial DNA is moved from one bacterium to another by a virus) or transfection (the introduction of foreign material into cells, which typically involves opening transient pores or ‘holes’ in the cell membrane, to allow the uptake of material. Transfection is frequently carried out by mixing a cationic lipid with the material to produce liposomes, which fuse with the cell membrane and deposit their contents inside.) Transformation is the genetic alteration of a cell resulting from the uptake and expression of foreign genetic material.

A “vector” is a replicon, such as plasmid, phage, bacterial artificial chromosome (BAC) or cosmid, to which another DNA segment (e.g. a foreign gene) may be incorporated so as to bring about the replication of the attached segment, resulting in expression of the introduced sequence. Vectors may comprise a promoter and one or more control elements (e.g., enhancer elements) that are heterologous to the introduced DNA but are recognized and used by the host cell. Alternatively, the sequence that is introduced into the vector retains its natural promoter that may be recognized and expressed by the host cell (Bormann et al., J. Bacteriol 1996; 178:1216-1218). A “replicon” is any genetic element (e.g., plasmid, chromosome, virus) that functions as an autonomous unit of DNA replication within a cell, i.e., capable of replication under its own control. In one embodiment, a vector of the present invention is a Bacterial Artificial Chromosome (BAC) shuttle vector that permits conjugation between e.g., Streptomyces and E. coli. A common way to insert one segment of DNA into another segment of DNA involves the use of specific enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct”. A common type of vector is a “plasmid”, which generally is a self-contained molecule of double-stranded DNA, usually of bacterial origin, that can readily accept additional (foreign) DNA and which can be readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence, which initiates, regulates, or otherwise mediates or controls the expression of the coding DNA. Promoter DNA and coding DNA may be from the same gene or from different genes, and may be from the same or different organisms. Recombinant cloning vectors will often include one or more replication systems for cloning or expression, one or more markers for selection in the host, e.g. antibiotic resistance, and one or more expression cassettes. Vector constructs may be produced using conventional molecular biology and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); F. M. Ausubel et al. (eds.), Current Protocols in Molecular Biology, John Wiley & Sons, Inc. (1994).

### General Description

The present invention provides a vector (pSBAC) that contains the following components: first, it contains the backbone of pCC1BAC, a Bacterial Artificial Chromosomal (BAC) vector, which has two replication origins (ori2 for initiation of single-copy replication and oriV for initiation of high-copy replication) and E. coli F factor-based partitioning system (ParA, ParB and ParC); second, it contains an origin of transfer (oriT) which permits the transfer of the vector from one bacterial cell to another, and this function is critical for conjugation which results in the transfer of nucleic acid from one prokaryotic cell to another through direct cell-to-cell contact; third, it also contains a φBT1 attP-int DNA fragment which encodes a site-specific recombination system that permit site-specific integration of the vector into the attB site of the recipient chromosome. With all these components, this vector is capable of harboring large DNA fragments, and transferring of cloned DNA from E. coli to streptomycetes, as well as integrating the cloned DNA into the Actinomycetes genome at the φBT1 attB locus. Because φBT1 has a different integration site than that of the widely used φC31 attP-int system, this vector represents an integration system that is different than the heavily exploited φC31 attP-int system. This vector system expands the repertoire of available integration conjugation vectors and avoids the potential detrimental effects caused by the φC31 attP-int system. (For example, the use of the φC31 attP-int system results in the reduced production of A47934, a glycopeptide antibiotic, in Streptomyces toyocaensis to 59% of the control strain. In addition, use of the φC31 attP-int system results in the reduced production of Spinosyns, a known agricultural insecticide, in Saccharopolyspora spinosa to 96% of the control strain. (Baltz, R. H. 1998. Trends Microbiol. 6:76-83; Matsushima, P. et al. 1994. Gene 146:39-45; Matsushima, P. and Baltz, R. H. 1996. Microbiology. 142:261-127)

Because the genes responsible for production of secondary metabolites are located in clusters that can be as large as 100 kb in size, this vector provides an important vehicle to clone those gene clusters as well as shuffle those large genetic segments between E. coli, in which the vector replicates autonomously, and various Actinomycetes hosts, in which it site-specifically integrates into the φBT1 attB loci in the chromosomes.

The plasmid rescue method has been used to isolate the chromosomal DNA adjacent to an inserted piece of DNA in various organisms. However, because of the limited cloning capacity of the vectors used for this purpose, this method was mainly used for cloning and identification of the adjacent region of the loci that the exogenous DNA inserted. Taking advantage of the ability of BAC vector to clone large DNA fragments, the present invention also provides a plasmid (BAC) rescue method using the pSBAC vector to clone biosynthetic gene clusters on large DNA fragments from streptomycetes. Successful implementation of this method will greatly facilitate the cloning of large DNA fragments, particularly, microbial secondary metabolic biosynthetic pathway, without sophisticated generation and screening of cosmid or BAC libraries. As a proof of principle, the vector was first transferred into Streptomyces coelicolor by conjugation from E. coli S17-1 (pSBAC). Apramycin-resistant transconjugants were recovered. Further analyses of two of them revealed that the vector specifically integrated into the φBT1 attB locus. The genomic DNA of those two were isolated and digested with different restriction enzymes, and the digested DNA was subjected to pulse field electrophoresis. Different portions of the DNA fraction of the electrophoresis were recovered. After self-ligation and transformation, the rescued DNA was introduced into E. coli strain, TranforMax EPI300. BAC DNA was isolated from several transformants and analyzed with restriction enzyme digestion and pulse field electrophoresis. The result demonstrated that 40 to 50 kb DNA fragment adjacent to the φBT1 attB site can be routinely cloned using this method. To prove that this method has a general application potential, 2 kb DNA fragment at the end of the actinorhodin gene cluster of S. coelicolor was amplified and cloned into the pSBAC vector that does not have the attP-int locus. Then, this construct was transformed into S. coelicolor and homologous recombination resulted in the single cross-over and site-specifically inserted the construct into the homologous region. The genomic DNA of the exconjugants was isolated and, the whole actionorhodin gene cluster was successfully rescued into a single E. coli clone using the above-described procedure. The φBT1 attP-int fragment was then introduced into the rescued clone to facilitate the subsequent integration of the gene cluster into the specific locus. Finally, the actinorhodin gene cluster was transferred into a mutated S. lividians strain in which the actinorhodin gene cluster had been deleted previously. Successful expression of the cloned actinorhodin gene cluster was demonstrated by the restoration of the production of actinorhodin.

### Methods for Cloning and Transfer of Large Nucleic Acid Fragments

One aspect of the invention provides for a vector for cloning or transfer of a large fragment of nucleic acid, e.g. a large DNA fragment comprising a whole or portion of a gene cluster from one prokaryotic organism to Actinomycetes. In one embodiment, the prokaryotic organism is a strain of E. coli. In other embodiments, the prokaryotic organism is the same or a different strain of actinomycetes, or any other organism known to those skilled in the art for use in transfer of nucleic acids from one organism to another, for example, from one prokaryotic organism to actinomycetes. The donor of the genetic material may be a prokaryotic or eukaryotic organism, known to those skilled in the art, for use in transfer of genetic material from one organism to another.

In one embodiment, the vector is a Bacterial Artificial Chromosome (BAC) vector. In one embodiment, the BAC vector is a shuttle BAC vector. In one embodiment, the shuttle BAC vector is an E. coli-Actinomycetes conjugative vector, designated pSBAC. In one embodiment, the vector further comprises a whole or portion of a gene cluster. While it is envisioned that the vector is a BAC vector, other vectors capable of transfer of large nucleic acid fragments are also envisioned for use. These include bacteriophage derived artificial chromosomes (PACs), as well as yeast artificial chromosomes (YACs).

Bacterial Artificial Chromosomes (BACs) and bacteriophage derived artificial chromosomes (PACs) have been employed for cloning of large DNA fragments. Moreover, BACs and PACs may have certain advantages over the traditional large DNA cloning system, the yeast artificial chromosomes (YACs). These include large carrying capacity (˜100-300 kb), high clonal stability, low rate of chimerism, and the ease with which they can be handled (Shizuya, H., Birren, B., Kim, U. J., Mancino, V., Slepak, T., Tachiiri, Y., and Simon, M. 1992. PNAS 89: 8794-8797; Ioannou, P. A., Amemiya, C. T., Games, J., Kroisel, P. M., Shizuya, H., Chen, C., Batzer, M. A., and de Jong, P. J. 1994. Nat. Genet. 6: 84-89; Marra, M. A., Kucaba, T. A., Dietrich, N. L., Green, E. D., Brownstein, B., Wilson, R. K., McDonald, K. M., Hillier, L. W., McPherson, J. D., and Waterston, R. H. 1997. Genome Res. 7: 1072-1084; Kelley, J. M., Field, C. E., Craven, M. B., Bocskai, D., Kim, U. J., Rounsley, S. D., and Adams, M. D. 1999. Nucleic Acids Res. 27: 1539-1546); Mozo, T., Dewar, K., Dunn, P., Ecker, J. R., Fischer, S., Kloska, S., Lehrach, H., Marra, M., Martienssen, R., Meier-Ewert, S. 1999. Nat. Genet. 22: 271-275; Hoskins, R. A., Nelson, C. R., Berman, B. P., Layerty, T. R., George, R. A., Ciesiolka, L., Naeemuddin, M., Arenson, A. D., Durbin, J., David, R. G. 2000. Science 287: 2271-2274; Osoegawa, K., Tateno, M., Woon, P. Y., Frengen, E., Mammoser, A. G., Catanese, J. J., Hayashizaki, Y., and de Jong, P. J. 2000. Genome Res. 10: 116-128; McPherson, J. D., Marra, M., Hillier, L., Waterston, R. H., Chinwalla, A., Wallis, J., Sekhon, M., Wylie, K., Mardis, E. R., Wilson, R. K. 2001; Nature 409: 934-941).

The development of bacterial artificial chromosomes (BACs) (Shizuya, H., et al., Proc. Natl. Acad. Sci. USA 89:8794 8797 (1992)) and P1-artificial chromosomes (PACs) (Ioannou, P. A., et al., Nature Genet. 6:84 89 (1994)) has greatly aided physical mapping projects and genomic sequencing. BACs and PACs have many advantages over yeast artificial chromosomes (YACs) for cloning large DNA inserts (Monaco, A. P., and Larin, Z., Trends Biotech. 12:280 286 (1994)), including the ease of preparation of microgram quantities of vector.

Gene expression from BACs and PACs has been demonstrated in cell culture systems (Wade-Martins, R., et al., Nature Biotech 18:1311 1314 (December 2000); Compton, S. H., et al., Gene Ther. 7:1600 1605 (2000); Kim, S. Y., et al., Genome Res. 8:404 412 (1998)) and in transgenic animal models (Antoch, M. P., et al., Cell 89:655 667 (1997); Yang, X. W., et al., Nature Genet. 22:327 335 (1999)). Wade-Martins et al. has developed a large insert shuttle vector for gene expression in human cells based on a fusion of the BAC and EBV episome technologies (Wade-Martins, R., et al., Nature Biotech 18:1311 1314 (December 2000); Wade-Martins, R., et al., Nucleic Acids Res. 27:1674 1682 (1999)). The vector was used for complementation of a cell culture phenotype by a genomic DNA transgene retained in human cells as an EBV-based episome (Wade-Martins, R., et al., Nature Biotech 18:1311-1314 (December 2000)). Extrachromosomal maintenance of the construct prevented DNA rearrangement often seen on construct integration. The vector described by Wade-Martins, supra, is based solely on EBV features.

Westphal, E. M., et al., Human Gene Therapy 9:1863 1873 (September 1998) and international Patent Publication WO 00/12693 to Vos et al. relate to a vector system for shuttling large genomic inserts from preexisting BAC or PAC libraries into human cells. The system utilizes a hybrid BAC-HAEC (human artificial episomal chromosome), which contains an F-based replication system as in BAC and the EBV oriP, for replication in human cells. Transcription of the human beta-globin gene (185 kb) was observed in vitro.

U.S. Pat. No. 6,143,566 to Heintz et al. relates to targeted BAC modification. This patent teaches a method for directly modifying an independent origin based cloning vector (such as a BAC, in one specific embodiment) in recombination deficient host cells, including generating deletions, substitutions, and/or point mutations in a specific gene contained in the cloning vector. The modified cloning vector may be used to introduce a modified heterologous gene into a host cell. In one Example presented, a modified BAC was inserted into a murine subject animal, and in vivo heterologous gene expression demonstrated. The methodology of this invention involves homologous recombination of the cloning vector with a conditional replication shuttle vector in a RecA host cell, wherein the conditional replication shuttle vector encodes a RecA-like protein. In a preferred embodiment, the vector is a BAC that has undergone homologous recombination with the temperature sensitive shuttle vector pSV1.RecA.

Sosio et al describe a BAC that can be shuttled between E. coli and a streptomycetes host where it integrates into the chromosome. They propose to construct a derivative of a BAC that can be stably maintained in the host by incorporating a gene cassette for site specific integration into the host chromosome. In particular, they used the φC31-attB-int system and tsr to confer resistance to thiostrepton. (Sosio et al. (2000), Nature Biotechnology, 18: 343-345). However, it has been reported that the integration of vectors into the φC31-attB site can cause detrimental effects on antibiotic production. (Baltz, et al. (1998), Trends Microbiol. 6:76-83). These reported reductions in antibiotic synthesis may be due to insertional mutagenesis or by integration into a pseudo-attB site or to some other factor.

The present invention provides for site-specific integration of the vector described herein into the recipient cell chromosome through use of a φBT1-attB integration system, thereby eliminating any of the detrimental effects associated with the φC31-attB-int system. This integration site is described by Gregory et al (Gregory et al. (2003), J. Bacteriol. 17:5320-5323).

Moreover, while the main advantage of using BAC vectors is for cloning and transfer of very large fragments of DNA, and for the stability of the clones, the amount of DNA recovery is very low. (Wild et al. (2002), Genome Res. 12:1434-1444). The present invention incorporates two origins of replication, the ori2 and the oriV into the vector, thus improving the overall yield of the cloned DNA of the present invention.

Clearly, there is a need in the art to simplify and enhance gene delivery systems for large capacity DNA cloning vectors, such as, e.g., BACs and PACs, so that DNA inserts (and in particular large DNA inserts) within the large capacity DNA cloning vector can be more easily transferred to cells. More particularly, there is a need for optimizing the ability to clone and transfer large DNA fragments, for example, a whole or a portion of a gene cluster from one prokaryotic organism to Actinomycetes.

As noted above, in order to expand the repertoire of cloning and transferring vehicles, as well as to circumvent the potential detrimental effects caused by φC31 attP-int system in some strains (Baltz, R. H. 1998. Trends Microbiol. 6:76-83), the inventors of the present application developed a new E. coli-Streptomyces shuttle BAC vector system by using the φBT1 attP-int locus. It has been reported that integration vectors based on φBT1-attP-int system not only have a broad host range, but also are compatible with the vectors based on φC31 attP-int system, therefore, provides an additional option for studying the molecular genetics of streptomycetes.

The shuttle vector, pSBAC was first introduced into the φBT1 attachment site of S. coelicolor by site-specific recombination. Regions of varied sizes flanking the attB site could be easily cloned into E. coli by the plasmid rescue strategy. Furthermore, the whole actinorhodin gene cluster was cloned using this method and subsequently expressed in a heterologous host.

A versatile E. coli-Streptomyces Shuttle Bacterial Artificial Chromosomal vector, pSBAC, was constructed to facilitate the cloning, transferring and heterologous expression of streptomycete secondary metabolites biosynthetic gene clusters. This vector is capable of harboring large DNA fragments, transferring of cloned DNA from E. coli to streptomycetes, as well as integrating the cloned DNA into streptomycete genome at the phage φBT1 attB locus. A plasmid rescue method using this vector has been developed to speed the process of cloning biosynthetic gene clusters for secondary metabolites from streptomycetes without sophisticated generation and screening of cosmids or BAC libraries. The cloned DNA can then be used for sequencing or heterologous expression of putative secondary metabolic gene clusters. As an example of the application, the actinorhodin gene cluster (act) from S. coelicolor was successfully rescued by this vector into a single E. coli clone and subsequently transferred into a mutated S. lividians strain in which the act gene cluster had been deleted. Successful expression of the cloned act gene cluster was demonstrated by the restoration of the production of actinorhodin.

Taking advantage of the ability of BAC vector to clone large DNA fragments, a plasmid (BAC) rescue method was developed using the new BAC vector to clone large DNA fragments from Stretptomycetes. First, the BAC vector with a piece of homologous sequence to the gene of interest was conjugated into the strain of interest. Homologous recombination will result in the insertion of the vector into the targeted locus. The genomic DNA of the exconjugants was then isolated and digested with restriction enzyme, then circularized and transformed into E. coli. The rescued plasmid was replicated in E. coli because of the presence of the origin of replication (ori) in the original vector and the transformants were selected on agar plate because of the resistance selection marker included in the vector. By taking advantage of the homologous recombination mechanism in different Streptomycetes, as well as the unique ability to clone large DNA fragment of the BAC vector, this method can recover significant length of sequences flanking the site-specific insertion site from a disruptant strain. Successful implementation of this method will greatly facilitate the cloning of large DNA fragments, particularly, microbial secondary metabolic biosynthetic pathway, without sophisticated generating and screening of cosmid or BAC libraries.

The present method utilizes a large capacity cloning vector, such as a BAC or a PAC. Although a BAC or PAC is a particularly preferred large capacity cloning vector, other large capacity cloning vectors known to those skilled in the art can also be used in the present invention. These include, e.g., cosmids (Evans et al., Gene 79:9 20 (1989)), yeast artificial chromosomes (YACS) (Sambrook, J., et al., A Molecular Cloning: A Laboratory Manual, 2.sup.nd Edition, Cold Spring Harbor Press, Cold Spring Harbor, N.Y. (1989), mammalian artificial chromosomes (Vos et al., Nature Biotechnology 15:1257 1259 (1997), human artificial chromosomes (Harrington et al., Nature Genetics 15: 345 354 (1997)), or viral-based vectors, such as, e.g., CMV, EBV, or baculovirus.

As used herein, the term “BAC” (Bacterial Artificial Chromosome) is intended to mean a cloning and sequencing vector derived from a bacterial chromosome into which a large genomic DNA fragment, typically up to 400 kb, can be inserted. BACs are based on the single-copy F-plasmid of E. coli and have been demonstrated previously to stably maintain human genomic DNA of >300 kb, and genomes of large DNA viruses, including those of baculovirus and murine cytomegalovirus (Shizuya, H., et al., Proc. Natl. Acad. Sci. USA 89:8794 8797 (1992); Luckow, V. A., et al., J. Virol. 67:4566 4579 (1993); Messerle, M., et al., Proc. Natl. Acad. Sci. USA 94:14759 14763 (1997).

As used herein, the term “PAC” is intended to mean a cloning and sequencing vector derived from a P1 bacteriophage into which a large genomic DNA fragment, typically up to 300 kb can be inserted. PACs are described in Ioannou, P. A., et al., Nature Genetics 6:84-89 (1994) and Sternberg et al., Proc. Natl Acad Sci USA 87:103 107 (1990).

BAC or PAC libraries, and especially those containing human genomic DNA as a result of the Human Genome Project, are readily available to those skilled in the art (See, e.g., Simon, M. I., Nature Biotechnol. 15:839 (1997)

Generally, and in the particular examples above, transforming host microorganisms with vectors carrying component polynucleotides is carried out with conventional techniques. In one embodiment, the vector is transferred to the host or recipient cell via conjugation between two organisms. In one embodiment, the vector may be transferred to a host cell or recipient cell via transfection methods. As used herein, the terms “transformation” and “transfection” are intended to refer to a variety of art-recognized techniques for introducing an exogenous nucleic acid sequence (e.g., DNA) into a host cell, including calcium phosphate or calcium chloride co-precipitation, DEAB-dextran-mediated transfection, lipofection, electroporation, optoporation, mechanical injection, biolistic injection, and the like. Suitable methods for transforming or transfecting host cells are found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989), and like laboratory manuals.

### Selection Markers

In one embodiment, the shuttle vector includes a “selection marker” or “selectable marker” that is functional in the cell that contains the nucleic acid of interest. This “selection marker”, upon expression, can allow the host cell to be distinguished from a cell that does not contain the nucleic acid of interest. For example, the term “selection marker” or “selectable marker” refers to the use of, or the inclusion of, a drug as a marker to aid in the cloning and identification of transformants, for example, genes that confer resistance to apramycin, neomycin, puromycin, hygromycin, DHFR, GPT, zeocin and histidinol are useful selectable or selection markers. Accordingly, cells containing a nucleic acid construct of the present invention may be identified by including a marker in the vector. Such markers would confer an identifiable change to the cell permitting easy identification of cells containing the vector. Generally, a selectable marker is one that confers a property that allows for selection. A positive selectable marker is one in which the presence of the marker allows for its selection, while a negative selectable marker is one in which its presence prevents its selection. An example of a positive selection marker is a drug resistance marker. In this embodiment, the positive selection marker is a gene that confers resistance to a compound, which would be lethal to the cell in the absence of the gene. For example, a cell expressing an antibiotic resistance gene would survive in the presence of an antibiotic, while a cell lacking the gene would not. For instance, the presence of a tetracycline resistance gene could be positively selected for in the presence of tetracycline, and negatively selected against in the presence of fusaric acid. Suitable antibiotic resistance genes include, but are not limited to, genes such as ampicillin-resistance gene, neomycin-resistance gene, blasticidin-resistance gene, hygromycin-resistance gene, puromycin-resistance gene, chloramphenicol-resistance gene, apramycin resistance gene and the like. In certain aspects, the negative selection marker is a gene that is lethal to the target cell in the presence of a particular substrate. For example, the thyA gene is lethal in the presence of trimethoprim. Accordingly, cells that grow in the presence trimethoprim do not express the thyA gene. Negative selection markers include, but are not limited to, genes such as thyA, sacB, gnd, gapC, zwJ, talA, taiB, ppc, gdhA, pgi, Jbp, pykA, cit, acs, edd, icdA, groEL, secA and the like. In one embodiment, the selectable marker gene is a gene that provides for apramycin resistance. (See GenBank accession number AJ414670)

In another embodiment, the selection marker gene may be a detection gene. Detection genes encode a protein that can be used as a direct or indirect label, i.e., for sorting the cells, i.e. for cell enrichment by FACS. In this embodiment, the protein product of the selectable marker gene itself can serve to distinguish cells that are expressing the selectable gene. In this embodiment, suitable selectable genes include those encoding green fluorescent protein (GFP), blue fluorescent protein (BFP), yellow fluorescent protein (YFP), red fluorescent protein (RFP), luciferase, β-galactosidase, all commercially available, i.e., Clontech, Inc.

Alternatively, the selectable marker gene encodes a protein that will bind a label that can be used as the basis of selection; i.e. the selectable marker gene serves as an indirect label or detection gene. For example, visually detectable markers are also suitable for use in the present invention, and may be positively and negatively selected and/or screened using technologies such as fluorescence activated cell sorting (FACS) or microfluidics. Examples of detectable markers include various enzymes, prosthetic groups, fluorescent markers, luminescent markers, bioluminescent markers, and the like. Examples of suitable fluorescent proteins include, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride, phycoerythrin and the like. Examples of suitable bioluminescent markers include, but are not limited to, luciferase (e.g., bacterial, firefly, click beetle and the like), luciferin, aequorin and the like. Examples of suitable enzyme systems having visually detectable signals include, but are not limited to, galactosidases, glucorimidases, phosphatases, peroxidases, cholinesterases and the like.

Another aspect of the invention provides a plasmid (BAC) rescue method using the pSBAC vector described above to clone large DNA fragments from streptomycetes. This method will greatly facilitate the cloning of large DNA fragments, particularly, microbial secondary metabolic biosynthetic pathway, without sophisticated generation and screening of cosmid or BAC libraries. As a first step, the pSBAC vector was first transferred into S. coelicolor by conjugation from E. coli S17-1 (pSBAC). Several apramycin-resistant transconjugants were selected and the genomic DNA from one of them were isolated and partially digested with ScaI. The digested DNA was subjected to pulse field electrophoresis and DNA fraction that corresponds to 40 to 50 kb was recovered, self-ligated, and then transformed into E. coli EPI300™. BAC DNA was isolated from several transformants and analyzed with restriction enzyme digestion and pulse field electrophoresis. The result demonstrated that 40 to 50 kb DNA fragment adjacent to the φBT1 attB site can be routinely cloned using this method.

### EXAMPLES

The following examples demonstrate certain aspects of the present invention. However, it is to be understood that these examples are for illustration only and do not purport to be wholly definitive as to conditions and scope of this invention. It should be appreciated that when typical reaction conditions (e.g., temperature, reaction times, etc.) have been given, the conditions both above and below the specified ranges can also be used, though generally less conveniently. All parts and percents referred to herein are on a weight basis and all temperatures are expressed in degrees centigrade unless otherwise specified.

### Example 1

### Construction of the pSBAC Vector and Development of the Plasmid Rescue Procedure

### Materials and Methods

### Bacterial Strains and Plasmids:

The Streptomyces coelicolor strain M145 and S. lividans TK24 were used in this work. S. lividans K4-441 (Ziermann and Betlach, 1999. BioTechniques 26:106-110) was used as a heterologous host for the expression of the Actinorhodin gene cluster. Escherichia coli strain EPI300™ (Epicentre, Madison Wis.) was used for cloning and amplification of the pSBAC vector and constructs derived from it. E. coli strain NovaBlue (Novagen, Madison, Wis.) was used for other cloning purpose. E. coli strain S17-1 was used for conjugation to introduce plasmids from E. coli to S. lividans. The plasmid pHLW3 was a BAC (Bacterial Artificial Chromosome) derived vector with oriT and Apramycin resistance gene. The pSBAC was derived from pHLW3 with the introduction of the attP-Int cassette of φBT1. Plasmids pHLW21, 22, 23, and 24 were rescued BAC clones from the strains derived from the conjugation of pSBAC into the φBT1 attachment site of S. coelicolor genome. Plasmids pHLW35 were derived from pHLW3 with the insertion of a 2-kb PCR product amplified from the S. coelicolor using the primer sets that corresponding to the 3′-end of the Actinorhodin gene cluster. Plasmids pHLW38, 39, 40, 41, and 42 were rescued BAC clones from the strains derived from the conjugation of pHLW35 into the Actinorhodin gene cluster of S. coelicolor. Finally, plasmid pHLW76 and 78 were derived by adding the attB-int cassette of φBT1 into plasmids pHLW41 and 42, respectively.

### Culture Media and Growth Conditions:

E. coli strains were grown in Luria-Bertani (LB) medium supplemented with either Apramycin (50 μg/ml) or Ampicillin (100 μg/ml). S. coelicolor and S. lividans strains were grown at 28° C. in MYM, R2YE or R4 media (Kieser, T. M. J., Bibb, M. J., Buttner, K. F., Chater, and D. A. Hopwood. (2000), Practical Streptomyces Genetics, University of Nottingham, Nottingham, UK). R6 medium used for conjugation was supplemented with Apramycin (50 μg/ml) and nalidixic acid (25 μg/ml).

### Molecular Genetic Techniques:

KOD Hot start DNA polymerase (Novgen) was used for PCR amplification following the manufacturer's instruction. Genomic DNA of Streptomycetes was isolated using the procedure described previously (Magarvey, N. A., Haltli, B., He, M., Greenstein, M., and Hucul J. A. 2006. Antimicrob Agents Chemosther. 50:2167-2177). Plasmid DNA was isolated using the Zappy plasmid miniprep kit (Zymo Research). BAC clones with large inserts were isolated using the BACMAX DNA purification kit (Epicentre). Transformation of plasmid into E. coli was performed using either NovaBlue competent cell or EPI300™ electrocompetent cells. Conjugation vectors were introduced into S. lividans TK24 or K4-114 using E. coli S17-1 harboring the intended plasmid as donor strain. Pulsed field electrophoresis was carried out using CHEF-DR III Pulse field electrophoresis system (Bio-Rad) following the manufacture's instruction. Briefly, the digested DNA was separated in 1% pulse filed agarose (Bio-Rad) in 0.5×TBE running buffer. Run the gel using the following parameters: 1 sec of initial switch time, 6 sec of final switch time, 120° included angle and 16 hr of run time at 14° C. under 6 volts/cm condition.

Construction of the pSBAC Vector.

The backbone of the pSBAC vector was amplified from plasmid pCC1 BAC (see SEQ ID NO: 8) (Epicentre, Madison, Wis.) using primer set pCC1BACFor: 5′-AGGGCTTCCCGGTATCAACAG-3′ (SEQ ID NO: 9) and pCC1BACRev: 5′-GGTTACTCCGTTCTACAGGTTAC-3′ (SEQ ID NO: 10). The origin of transfer region (oriT) and Apramycin resistance gene, together with the multiple cloning site were amplified from plasmid pBWA2 (Magarvey, N. A., Haltli, B., He, M., Greenstein, M., and Hucul J. A. 2006. Antimicrob Agents Chemosther. 50:2167-2177) using the primer set: pB1: 5′-TCAGGCCTTCGCCACCTCTGACTTGAGC-3′ (SEQ ID NO: 11) and pB2: 5′-ATAGGCCTCAGTGAGGCACCTATCTCAG-3′ (SEQ ID NO: 12). The 6.5-kb PCR amplified from the first primer set and the 4-kbPCR product amplified from the second primer set were ligated together to produce plasmid pHLW3. Then, the 2-kb DNA fragment containing the attP-int of φBT1 was synthesized (Celtek-genes, Nashville, Tenn.) and introduced into the unique ScaI site of pHLW3 to give the final construct pSBAC.

### Plasmid Rescue Procedure.

The exconjugants obtained from the conjugation of S17-1 (pSBAC) into S. coelicolor were grown in MYM liquid medium supplemented with Apramycin (50 μg/ml). The genomic DNA was partially digested with ScaI, the digested DNA then was separated using pulsed field electrophoresis. The DNA fraction corresponding to 40-60 kb was excised and electroeluted from the gel by electrophoresis. The electroeluted DNA was self-ligated using Fast-link DNA ligation kit (Epicentre Bio). The ligation mixture was then desalted in 1% agarose with 1.8% glucose, and 1 μl of the final ligation mixture was used to electroporate E. coli EC100 competent cell. Several transformants were randomly selected and the plasmid DNA was isolated. The plasmid DNA was digested with different restriction enzymes to check the size of the rescued plasmids and some of them were subjected to sequencing. In order to rescue the act gene cluster from S. coelicolor, 2 kb PCR product that corresponding to the 3′-end of the gene cluster (corresponding to genomic sequence position: 170311 to 172280 of GenBank Accession number AL939122.1) was amplified using primer set Scres1: 5′-CTGAATTCCGACGTCGACGTGTACTACCTGACCC-3′ (SEQ ID NO: 13) (EcoRI site underlined) and Scres2: 5′-GAAAGCTTACGCGATAGCGATTGCCGTTGTCGTC-3′ (SEQ ID NO: 14) (HindIII site underlined).

This 2-kb PCR product was digested with EcoRI and HindIII, and then ligated to the corresponding site of pHLW3. The resulting plasmid pHLW35 was then introduced into S. coelicolor by conjugation. The specific integration of the plasmid into the ACT gene cluster was first confirmed in several excojugants by PCR analyses. Then the act gene cluster was rescued using the above described procedure. The existence of the whole act gene from those clones were confirmed by PCR analysis using the primer set pACT1: 5′-CAGTTCGGCGGGCCGGACGTACTGGGCCTC-3′ (SEQ ID NO: 15) and pACT2: 5′-CGGCGAGGGCTTCCGGTCCGCCGTGGCAGT-3′ (SEQ ID NO: 16) that corresponding to the very beginning of the act gene cluster (corresponding to genomic sequence position: 147001 to 147640 of GenBank accession number AL939122)

### Preparation of Extracts and Mass Spectrometry:

To analyze the production of Actionorhodin in various strains, the appropriate strains were grown in 10 ml of R2YE at 28° C. for 5 days. 1 ml culture was taken from each sample and extracted with equal volume of ethyl acetate-methanol (95:5 [vol:vol]). The extracts were then dried down by speed vacuum and re-suspended in 200 μl methanol for liquid chromatography-mass spectrometry (LC/MS) analyses. The analyses were carried out on an Agilent 1100 system coupled with a LCQ Deca mass spectrometer. The samples were chromatographed with 5% to 95% MeCN/0.025% formic acid in H₂O/0.025% fomic acid over 15 min on an YMC ODS S-3 column (2.0×100 mm).

### Results:

Construction of the E. coli-streptomycetes BAC Vector pSBAC.

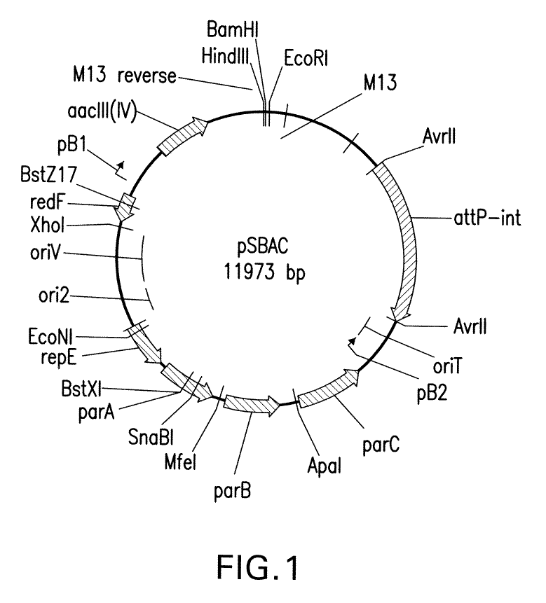

An E. coli-streptomycetes conjugative BAC vector, pSBAC (FIG. 1), was constructed to facilitate cloning of large DNA fragments and subsequent transferring those fragments from E. coli to streptomycetes. This vector contains the backbone of pCC1BAC, a Bacterial Artificial Chromosomal (BAC) vector, which has two replication origins (ori2 for initiation of single-copy replication and oriV for initiation of high-copy replication) and E. coli F factor-based partitioning system (ParA, ParB and ParC) (Wild, J., Hradecna, Z., and Szybalski W. 2002. Genome Res. 12:1434-1444). The pSBAC vector also contains an origin of transfer (oriT) which permits the transfer of the vector from one bacterial to another, and this function is critical for conjugation which results in the transfer of nucleic acid from one prokaryotic cell to another through direct cell-to-cell contacts; third, it also contains a φBT1 attP-int DNA fragment which encodes a site-specific recombination system that permit site-specific integration of the vector into the attB site of the recipient chromosome. With all these components, this vector is capable of harboring large DNA fragments, and transferring of cloned DNA from E. coli to streptomycetes, as well as integrating the cloned DNA into the streptomycetes genome at the φBT1 attB locus. Because φBT1 has a different integration site than that of the widely used φC31 attP-int system, this vector represents an integration system that is different, yet compatible with the heavily exploited φC31 attP-int system. Construction of this vector system expands our repertoire of available integration conjugation vectors and avoids the potential detrimental effects caused by the φC31 attP-int system. Because the genes responsible for production of secondary metabolites are located in clusters that can be as large as 100 kb in size, this vector provides an important vehicle to clone those gene clusters as well as shuffle those large genetic segments between E. coli, in which the vector replicates autonomously, and various streptomycetes hosts, in which it site-specifically integrates into the φBT1 attB loci in the chromosomes.

Development of Plasmid Rescue Method Using pSBAC Vector.

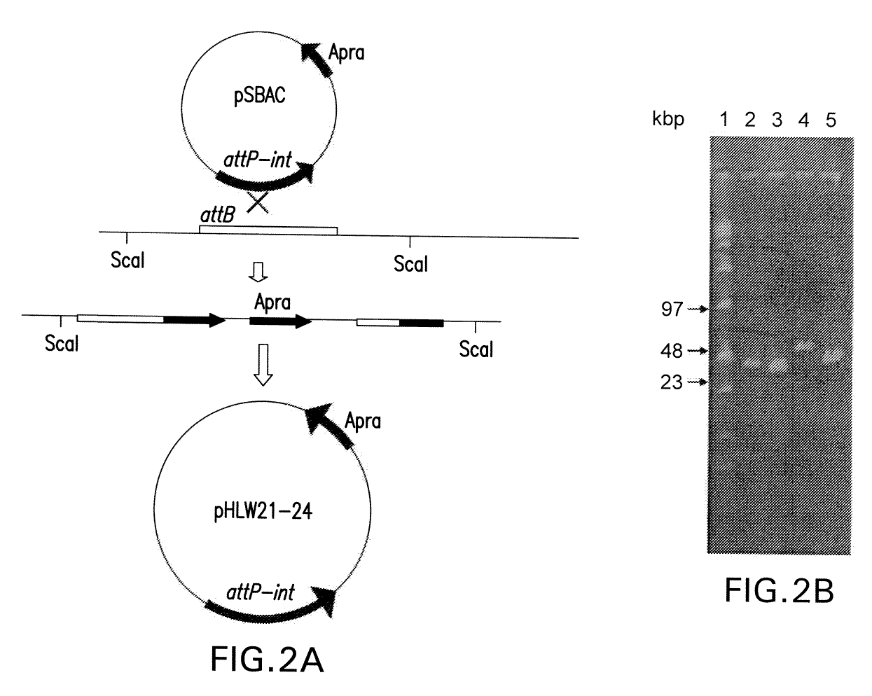

The plasmid rescue method has been used to isolate the chromosomal DNA adjacent to an inserted piece of DNA in various organisms (Weinrauch, Y., and Dubnau, D. 1983. J Bact. 154:1077-1087; Kiessling, U., Platzer, M., and Strauss, M. 1984. Mol Gen Genet. 193:512-519.; McMahon T. L., Wilczynska, Z., Barth, C., Fraser, B. D., Pontes, L., and Fisher P. R. 1996. Nucleic Acids, Res. 24:4096-4097). However, because of the limited cloning capacity of the vectors used for this purpose, this method was mainly used for cloning and identification of the adjacent region of the loci that the exogenous DNA inserted. Taking advantage of the ability of BAC vector to clone large DNA fragments, a plasmid (BAC) rescue method was developed using the pSBAC vector to clone large DNA fragments from streptomycetes. Successful implementation of this method will greatly facilitate the cloning of large DNA fragments, particularly, microbial secondary metabolic biosynthetic pathway, without sophisticated generation and screening of cosmid or BAC libraries. As a first step to prove the principle, the pSBAC vector was first transferred into S. coelicolor by conjugation from E. coli S17-1 (pSBAC). Several apramycin-resistant transconjugants were selected and the genomic DNA from one of them were isolated and partially digested with ScaI. The digested DNA was subjected to pulse field electrophoresis and DNA fraction that corresponds to 40 to 50 kb was recovered, self-ligated, and then transformed into E. coli EPI300™. BAC DNA was isolated from several transformants and analyzed with restriction enzyme digestion and pulse field electrophoresis (FIG. 2). FIG. 2a outlines the strategy used for plasmid rescue using the pSBAC vector. FIG. 2b shows the results of the pulse-field gel electrophoresis analysis of four of the rescued clones digested with HindIII. (lane 1: pulse marker; lanes 2-5: rescued clones pHLW21, 22, 23 and 24). Apra: apramycin resistance marker. The result demonstrated that 40 to 50 kb DNA fragment adjacent to the φBT1 attB site can be routinely cloned using this method.

Cloning of the Actinorhodin Gene Cluster by Plasmid Rescue and Heterologous Expression of this Gene Cluster in a Surrogate Host.

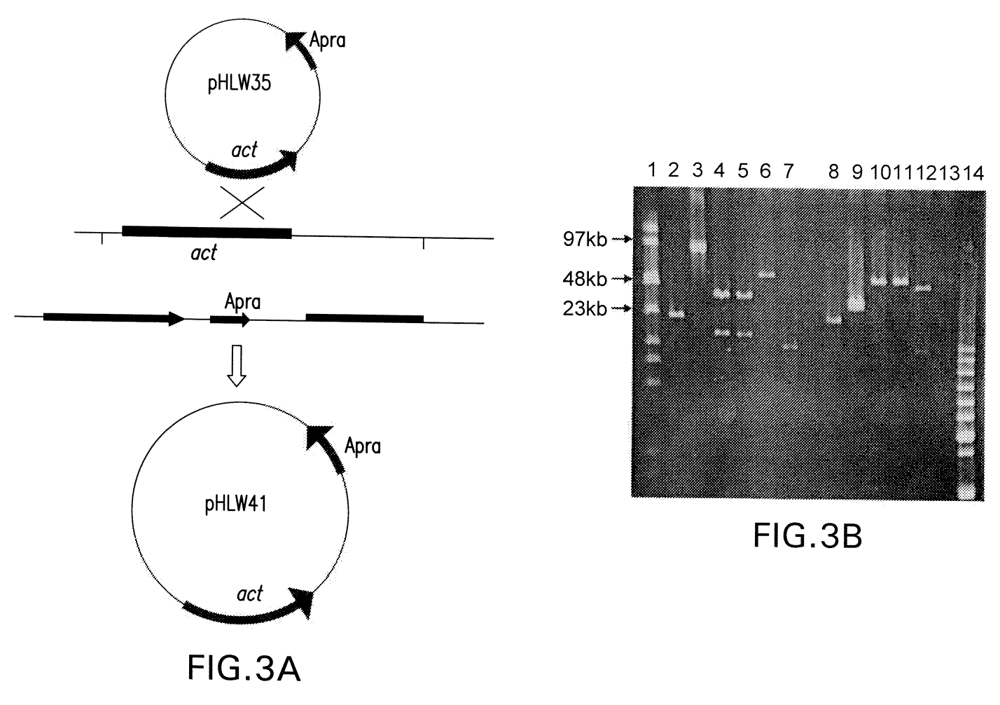

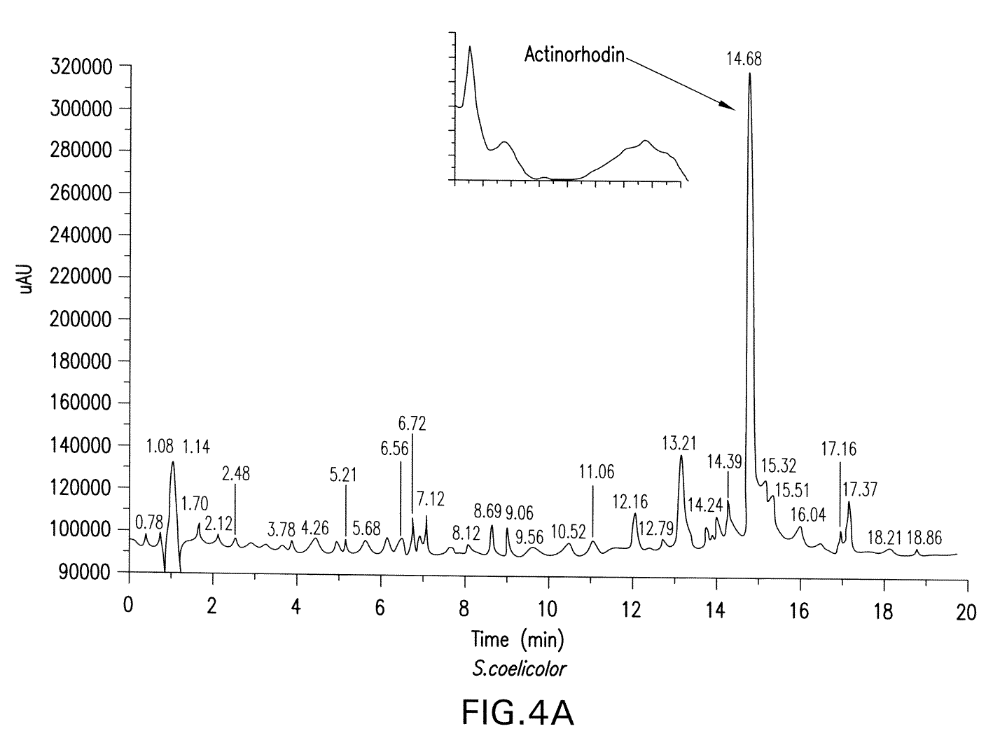

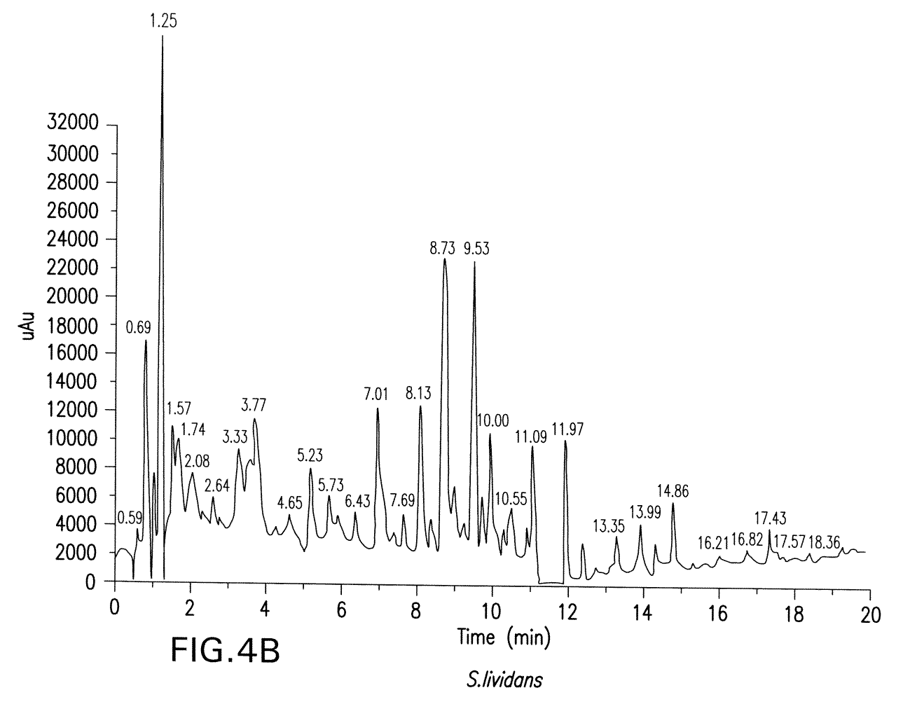

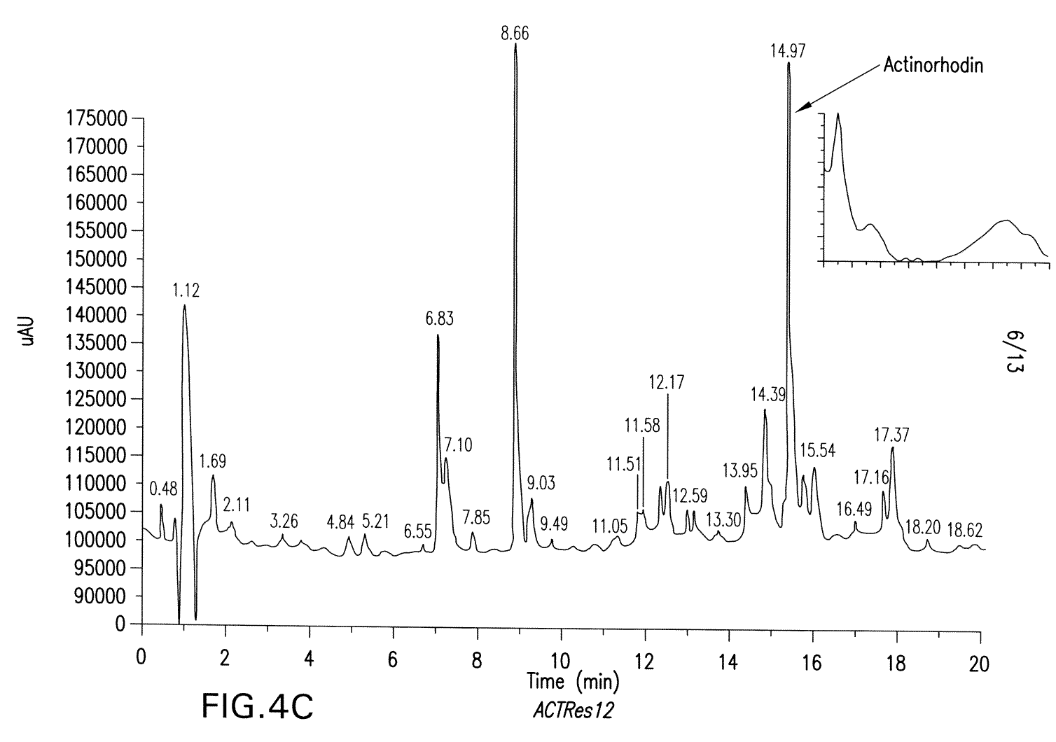

To further extend the application of the plasmid rescued method developed using pSBAC and to prove that this method has a general application potential and can be used to clone large gene clusters of interest, the well-characterized Actinorhodin gene cluster was chosen for further cloning and expression study. FIG. 3a shows the schematic representation of the strategy used for rescue of the act gene cluster from S. coelicolor and 3b the analysis of the rescued clones. The 2 kb DNA fragment corresponding to the end of the Actinorhodin gene cluster of S. coelicolor was amplified using primer set Scres1 and Scres2, then cloned into the vector pHLW3, which is different from pSBAC in that it does not have the attP-int locus. The resulting construct was then transformed into S. coelicolor and homologous recombination resulted in the single cross-over and site-specifically inserted the construct into the homologous region (FIG. 3a). After confirming the site-specific integration in some of the exconjugants by PCR analysis (data not shown), the genomic DNA from one of these strains was isolated and the genomic DNA was mechanically sheared to smaller pieces. The resulting DNA was then separated by pulsed-field electrophoresis and 40 to 50 kb DNA fraction was recovered. FIG. 3b shows the results of the pulsed-field gel electrophoresis analysis of five of the rescued clones that potentially contain the whole act gene cluster (lane 1: pulse marker; lanes 2-7: clone pHLW38, 39, 40, 41 and 42 digested with EcoR1; lanes 8-13, the above clones digested with HindIII; lane 14: 1 kb DNA ladder). The recovered DNA was blunt-ended using End-It™ DNA end repair kit (Epicentre), then self-ligated and transformed into E. coli EPI300™ electrocompetent cells. Numerous transformants were subject to PCR analyses to select the clones that contain both the beginning and the ending of the Actinorhodin gene cluster. The confirmed clones were the clones that potentially contain the whole Actinorhodin gene cluster because of the presence of both ends of the gene cluster. The φBT1 attP-int fragment was then introduced into the AvrII site of the rescued clone to facilitate the subsequent integration of the gene cluster into the specific attB attachment locus. Finally, the clone with the whole Actinorhodin gene cluster was transferred into S. lividans strain K4-114 in which the Actinorhodin gene cluster had been deleted previously. Exconjugants were selected from R6 plate supplemented with apramycin and Nalidixic acid and re-streaked onto MYM agar plate supplemented with apramycin and Nalidixic acid. After incubation at 28° C. for 5 days, most of the streaked colonies showed red-blue color and indicated the successful introduction and expression of the Actinorhodin gene cluster in the new host (data not shown). The production of actinorhodin was further characterized by examining the production of the blue pigment by the representative clone grown on either R2YE agar plate or liquid medium. (Bystrykh, L. V., Fernandez-Moreno, M. A., Herrema, J. K., Malpartida, F., Hopwood, D. A., and Dijkhuizen, L. 1996. J Bacteriol 178: 2238-2244; Floriano, B., and Bibb, M. 1996. Mol Microbiol 21: 385-396.). Finally, LC/MS analysis confirmed the production of actinorhodin in this clone (FIG. 4). The restoration of the production of actinorhodin demonstrated the cloned Actinorhodin gene cluster was successfully expressed in the heterologous host. Interestingly, because S. lividans TK24, the parental strain of K4-114, does not produce actinorhodin under normal growth condition (Hopwood, D. A., Malpartida, F. and Chater, K. F. 1986. In regulation of secondary metabolite formation, Kleinkauf, H. et al. (eds.) VCH, Weinheim Germany, pp. 23-33), the detection of actinorhodin in those exconjugants suggested that the rescued plasmid clone used for conjugation contained the regulatory element to activate actinorhodin production in S. lividans and this result is consistent with previous study showed that the actII region of the act gene cluster in S. coelicolor contains positive regulatory sequence that could induce the expression of act gene cluster in S. lividians (Fernandez-Moreno, M. A., Caballero, J. L., Hopwood, D. A. and Malpartida, F. 1991. Cell. 66:769-780).

A novel E. coli-Streptomycetes shuttle vector, pSBAC, was constructed and this vector can be used for direct cloning of small, medium or large gene clusters, transferring of the cloned DNA from E. coli into different soil bacteria streptomycetes strains, and integrating the cloned DNA into the φBT1 attB site of the recipient chromosome. This vector represents an integration system that is different, yet compatible with the widely-used φC31 attP-int system and expands the repertoire of available integration conjugation vectors. The plasmid (BAC) rescue method developed here can be used for cloning large DNA fragments directly from gene disruption transformants of streptomycetes without generation and screening of cosmid or BAC libraries. It only involves several simple steps of DNA handling and minimizes the procedure that would compromise the integrity of genomic DNA that is essential for building high quality BAC libraries.

### Example 2

### Rapid Cloning and Heterologous Expression of Meridamycin Biosynthetic Gene Cluster Using pSBAC

Meridamycin and its naturally occurred analog 3-normeridamycin are non-immunosuppressive, FKBP12-binding macrocyclic polyketides with potent neuroprotective activity in dopaminergic neurons. The biosynthetic gene cluster of meridamycin has been cloned from Streptomyces sp. NRRL 30748 and was located on several overlapping cosmids (He et al., Gene, (2006) August 1; 377:109-18). The entire gene cluster is ˜90 kb, containing large transcriptional units encoding a total of 15 type I polyketide synthase modules, 1 NRPS module, 1 cytochrome P450 monooxygenase and several regulatory and transportation proteins. The giant polyketide synthetase complex comprises three large subunits designated as MerA, MerB and MerC.

In the present invention, the mer gene cluster was cloned in a pSBAC vector to facilitate the transfer and expression in a heterologous host. More particularly, by using pSBAC, the whole meridamycin biosynthetic gene cluster (˜97 kb) was cloned into a single E. coli clone and then transferred into Streptomyces lividans for heterologous expression. Although the original mer promoter was able to drive the transcription of the whole gene cluster in the heterologous hosts, the production of meridamycin was only detectable by mass spectrometry when the original promoter was replaced with ermE* promoter. Semi-quantitative RT-PCR revealed that the promoter efficiency and transcription level of mer gene cluster is the foremost factor that affects the production of meridamycin and its analogs in the new host. Feeding precursors for ethymalonyl-CoA also enhanced the production of meridamycin and its analogs.

### Materials and Methods

### Bacterial Strains and Plasmids

Various strains and plasmids used in this study are summarized in Table 1 below.

TABLE 1
Bacterial strains and plasmids used in this study.

| | | Source/ |
|---|---|---|
| Strain/plasmid | Relevant genotype/comments | Reference |
| E. coli | | |
| EPI300 ™ | F⁻ mcrA D(mrr-hsdRMS-mcrBC) trfA | Epicentre, Madison, WI |
| | host for cloning and amplification of | |
| | various BAC vector and constructs | |
| | derived from it | |
| S17-1 | E. coli host for transferring various | Simon et al., 1983 |
| | plasmids into Streptomyces via | |
| | conjugation. | |
| ET12567(pUZ8 | E. coli host for transferring various | Paget et al. 1999 |
| 002) | plasmids into Streptomyces via | |
| | conjugation. | |
| S. coelicolor | | |
| A3(2) strain | SCP1⁻, SCP2⁻, Pgl⁺ | Kieser et al., 2000 |
| M145 | | |
| SCACT1 | Derivative strain of M145 with pHLW35 | This study |
| | integrated at the end of act gene cluster | |
| S. lividans | | |
| TK24 | RpsL(Sm^{r}) Act⁺Red⁺ | John Innes Centre, |
| K4-114 | str-6, SLP2⁻, SLP3⁻, Δact::ermE | Norwich, UK |
| | Streptomyces host for the expression of | Ziermann et al., 1999 |
| | the act gene cluster | |
| ACTRes12 | Derivative strain of K4-114 with rescued | This study |
| | act gene cluster inserted at the φBT1 attB | |
| | locus | |
| Streptomyces | Original meridamycin producing strain. | He et al. 2006 |
| sp. NRRL30748 | | |
| HL30_2 | S. lividans TK24 with mer gene cluster | This study |
| | integrated into chromosome | |
| HL 30_K3 | S. lividans K4-114 with mer gene cluster | This study |
| | integrated into chromosome | |
| E3 | S. lividans TK24 with mer gene cluster | This study |
| | under ermE* promoter integrated into | |
| | chromosome | |
| E7 | S. lividans K4-114 with mer gene cluster | This study |
| | under ermE* promoter integrated into | |
| | chromosome | |
| Plasmids | | |
| pCC1BAC | copy control BAC cloning vector | Epicentre, Madison, WI |
| pBWA2 | aacIII(IV), oriT | Magarvey et al., 2006 |
| pHLW3 | aacIII(IV), oriT, backbone of pCC1BAC | This study |
| pSBAC | aacIII(IV), oriT, attP-int, backbone of | This study |
| | pCC1BAC | |
| pHLW35 | pHLW3 with 2kb act EcoRI-HindIII DNA | This study |
| | fragment | |
| PHLW38-42 | pHLW35 derivatives rescued from S. coelicolor | This study |
| | SCACT1 | |
| pHLW76 | pHLW41 with attP-int | This study |
| pHLW30 | pSBAC with ~90kb DNA insert containing | This study |
| | whole mer gene cluster | |
| pHLW70 | pSE34 derivative containing the DNA | This study |
| | fragment downstream of merP under | |
| | ermE* promoter | |
| pHLW71 | pHLW70 with oriT | This study |

### Culture Media and Growth Conditions

E. coli strains were grown in Luria-Bertani (LB) medium supplemented with either apramycin (50 μg/ml) or ampicillin (100 μg/ml). S. coelicolor and S. lividans strains were grown at 28° C. in MYM, R2YE (Kieser, T., Bibb, M. J., Buttner, M. J., Chater, K. F., and Hopwood, D. A. (Eds.), 2000. Practical Streptomycetes genetics. The John Innes Foundation, Norwich, England.) or FKA media (Leaf, T., Burlingam, M., Desai, R., Regentin, R., Woo, E., Ashley, G., and Licari, P. 2002, J. Chem. Tech. Biotech. 77:1122-1126). R6 medium (Magarvey, N. A., Haltli, B., He, M., Greenstein, M., and Hucul J. A. 2006, Antimicrob. Agents Chemother. 50:2167-2177) used for conjugation was supplemented with apramycin (50 μg/ml) and nalidixic acid (25 μg/ml).

### DNA Manipulation

KOD Hot start DNA polymerase (Novgen, San Diego, Calif.) was used for PCR amplification following the manufacturer's instruction. Genomic DNA of Streptomycetes was isolated using the procedure described previously (Magarvey, N. A., Haltli, B., He, M., Greenstein, M., and Hucul J. A. 2006. Antimicrob. Agents Chemother. 50:2167-2177). Plasmid DNA was isolated using the Zappy plasmid miniprep kit (Zymo Research, Orange, Calif.). BAC clones with large inserts were isolated using the BACMAX DNA purification kit (Epicentre, Madison, Wis.). Transformation of plasmid into E. coli was performed using either NovaBlue competent cell or EPI300™ electrocompetent cells. Conjugation vectors were introduced into S. lividans TK24 or K4-114 using E. coli S17-1 or ET12567 (pUZ8002) harboring the intended plasmid as donor strain. Pulsed field electrophoresis was carried out using CHEF-DR III Pulse field electrophoresis system (Bio-Rad, Hercules, Calif.) following the manufacture's instruction. Briefly, the digested DNA was separated in 1% pulse filed agarose (Bio-Rad) in 0.5×TBE running buffer. Gel was run using the following parameters: 1 second of initial switch time, 6 seconds of final switch time, 120° included angle and 16 hr of run time at 14° C. under 6 volts/cm condition.

Rapid Cloning of Meridamycin Biosynthesis Gene Cluster Using pSBAC

Streptomyces sp. NRRL 30748 was grown in TSBC liquid medium for 72 h at 28° C. The mycelia was collected and washed with de-ionized water. Preparation of the genomic DNA plug was carried out following the instruction manual for CHEF Genomic DNA Plug Kits (Bio-Rad). Briefly, the mycelium pellet was re-suspended in Cell Suspension buffer and then embedded into CleanCut agarose using the plug mold. The solidified agarose plugs were then treated with lysozyme and subsequently with proteinase K. The plugs were washed twice with 1× Wash Buffer before treated with 1 mM PMSF to inactivate residual Proteinase K. Finally the plugs were washed thoroughly and stored in 1× Wash buffer at 4° C. until use. Pretreatment and restriction digestion of the DNA plugs were performed using the protocol described by Peterson et al (Peterson, D. G., Tomkins, J. P., Frisch, D. A., Wing, R. A., and Paterson, A. H. 2000, Journal of Agricultural Genomics, 5:1-100.). The DNA plugs were then chopped and digested by restriction enzyme Mfel at 37° C. for 2 hr. The digestion reaction was stopped by adding EDTA to the final concentration of 50 mM. The small pieces of the digested DNA plugs were then subject to pulsed-field electrophoresis in 1% pulse filed agarose (Bio-Rad) in 0.5×TBE running buffer using the following parameters: initial switch time=1.0 sec, final switch time=50.0 sec, run time=16 hr, volts/cm=6, included angle=120°, temperature=14° C. The DNA fraction corresponding to 90 to 110 kb was excised, eluted from the gel by electro-elution. The eluted DNA was precipitated and concentrated before ligate to EcoRI digested pSBAC vector using Fast-link DNA ligation kit (Epicentre Bio). The ligation mixture was then desalted in 1% agarose containing 1.8% glucose, and 1 μl of the final ligation mixture was used to electroporate E. coli EPI300 competent cell. About 300 recombinant colonies have been screened using DNA probes derived from the sequences flanking the mer gene cluster, resulting the identification of pHLW30, which contains the whole meridamycin biosynthetic gene cluster.

### RNA Isolation and RT-PCR Analysis

Total RNA from different Streptomycete strains was isolated using the method described by Van Dessel, W. V (2004) (Van Dessel, W., Van Mellaert, L., Geukens, N., Lammertyn, E and Anné, J. 2004. J. Microbiol. Methods 58:135-137) with modification. Briefly, 3 ml culture from 72 hr growth was collected and 2 vol of RNA protect Reagent (Qiagen) was added immediately and stand at room temperature for 5 min, then centrifuge to get the mycelium pellets. The pellets were treated with 1 ml of 5 mg/ml lysozyme for 1 hr at 37° C., then extracted with phenol:chloroform (5:1; pH4.5) and precipitated with 2 ml of ethanol, 250 μl of 1M Tris (pH8.0) and 100 μl 5M NaCl. The precipitated RNA was washed with 80% ethanol once, dry down and resolved in 100 ul RNA storage buffer (Ambion, Austin, Tex.). The DNA contamination was eliminated with DNase 1 digestion (Ambion) and re-purified repeating the above procedure. The primer sets used for RT-PCR were: RT1: 5′-GCGCGGACCGAGCCCTACGAC-3′ (SEQ ID NO: 19), RT2: 5′-CCCCCGGCCCTCCAGCAGATG-3′ (SEQ ID NO: 20) for amplification of the 5′-end of the mer gene cluster; Primers 16sFor: 5′-GGTTACCTTGTTACGACTT-3′ (SEQ ID NO: 21) and 16sRev: 5′-AGAGTTTGATCCTGGC TCAG-3′ (SEQ ID NO: 22), were used as an internal control to ensure the equal amount of total RNA was present in each sample. Semi-quantitative RT-PCR was similar to the method described previously (Noonan, K. E., Beck, C., Holzmayer, T. A., Chin, J. E., Wunder, J. S., Audrulis, I. L., Gazdar, A. F., William, C. L., Griffith, B., Hoff, D. D. V and Roninson, I. B. 1990. Proc. Natl. Acad. Sci. USA 87:7160-7164.), except one-step RT-PCR kit (Qiagen) was used following the instruction manual. Cycle numbers and template amount were carefully calibrated to ensure that the RT-PCR was carried out within the exponential phase of amplification.

Change the Original Mer Promoter with ermE* Promoter

The 2 kb PCR product corresponding to the immediate downstream of the original/native MerP promoter was amplified using primer set P1: GCTCTAGAGTGGGGAATTCAGGCGCACCC (SEQ ID NO: 23) (XbaI site was underlined) and P2: AGCAAGCTTGGGGACTCCGGTGGAGCCGGA (SEQ ID NO: 24) (HindIII site was underlined). The PCR product was purified and digested with XbaI and HindIII, then cloned into the corresponding sites of plasmid pSE34 (Schmitt-John, T., and Engels, J. W. 1992. Appl. Microbiol. Biotechnol. 36:493-498.) to produce plasmid pHLW70. In order to mobilize this vector, a 1.2 kb oriT sequence was amplified from plasmid pBWV2 (Magarvey, N. A., Haltli, B., He, M., Greenstein, M., and Hucul J. A. 2006. Antimicrob. Agents Chemother. 50:2167-2177) using primer set: oriTFOR: 5′-TTGCCTTGCTCGTCGGTGA-3′ (SEQ ID NO: 25) and oriTREV: 5′-CGCACGATATACAGGATTTGC-3′ (SEQ ID NO: 26). The 1.2 kb PCR product was purified and its 5′-end was phosphated by T4 PNK. The final product was cloned into the blunted BstBI site of pHLW70 and produced plasmid pHLW71. The plasmid was conjugated into heterologous hosts HL30-2 and HL30-K3. Numerous exconjugants were selected for further colony PCR analyses using the primer set: ErmE1: 5′-GGGGAGGATCTGACCGACGCGG-3′ (SEQ ID NO: 27); ErmE2: 5′-CGTTGGCGTGGACCCATGTGG CG-3′ (SEQ ID NO: 28), and ErmE3: 5′-AGCCCGACCCGAGCACGCGCCG-3′ (SEQ ID NO: 29); ErmE4: 5′-CGGGCG CACAGCTCGCGCAGATCGT-3′ (SEQ ID NO: 30) to select the strains that contain the intended single cross-over which resulted in the placement of ermE* promoter in front of the mer gene cluster. The PCR product was cloned and sequenced to confirm the site-specific integration. The final strains E3 and E7 contain the mer gene cluster under the control of ermE* promoter were derived from HL30-2 and HL30-K3, respectively.

### Preparation of Extracts and Mass Spectrometry

For 15 ml of fermentation culture, 1 g of HP20 resin was added and incubated at 28° C. for 2 hr with 200 rpm shaking. The mycelium, together with HP20 was collected by centrifugation. 10 ml of Ethyl acetate:Methanol (95:5) was added and vortex for 5 min. Centrifuge to separate layers and the upper ethyl acetate layer was collected and dried down using SpeedVac, then re-dissolved in 500 μl methanol. Meridamycin and its analogs were detected using liquid chromatography/mass spectrometry (LC/MS) on an Agilent 1100 system using electrospary ionization in negative ion mode. The samples were eluted with 65%-90% B in A over 15 min with a flow rate of 0.3 ml/ml on a Agilent Zorbox SB-C18 column (A=5 mM ammonia acetate; B=methanol). To prepare samples for high-resolution and accurate mass measurement (HRMS), the crude extract was fractionated on a LCQ Mass spectrometer using a linear gradient of 5% to 95% acetonitrile in water on an YMC-ODS 4.6×150 mm 5 μm column. Fractions that corresponding to the meridamycin and 3-Nor-meridamycin were collected, dried down and resuspended in 30 μl MeOH and subjected to HRMS. High resolution mass spectra (HRMS) were obtained using a Bruker Daltonics (Billerica, Mass.) APEX II FTICR mass spectrometer equipped with an actively shielded 9.4 Tesla superconducting magnet (Magnex Scientific Ltd., UK), an external Bruker Apollo ESI source, and a Synrad 50W CO₂ CW laser. A detailed description of this instrument and its performance has been published previously (Palmblad, M., Hakansson, K., Hakansson, P., Feng, X., Cooper, H. J., Giannakopulos, A. E., and Derrick, P. J. 2000, Eur. J. Mass. Spectrom. 6:267-275). Nanoelectrospray was employed due to the very limited quantities of samples. About 5 μl sample was loaded into nanoelectrospray tip with conductive coating (New Objective, Woburn, Mass.) and mixed with the pre-loaded same amount of methanol containing 1% formic acid. A high voltage about −800 V was applied between the nanoelectrospray tip and the capillary. Mass spectra were calibrated externally using Agilent ES tuning mix. Bruker Xmass software (Versions 7) was used for data acquisition and analysis, including the calculations for predicted masses. The errors are the differences between the experimental and predicted values expressed in mDa.

### Results and Discussion:

pSBAC Cloning of the Meridamycin Biosynthetic Gene Cluster into a Single E. coli Clone.

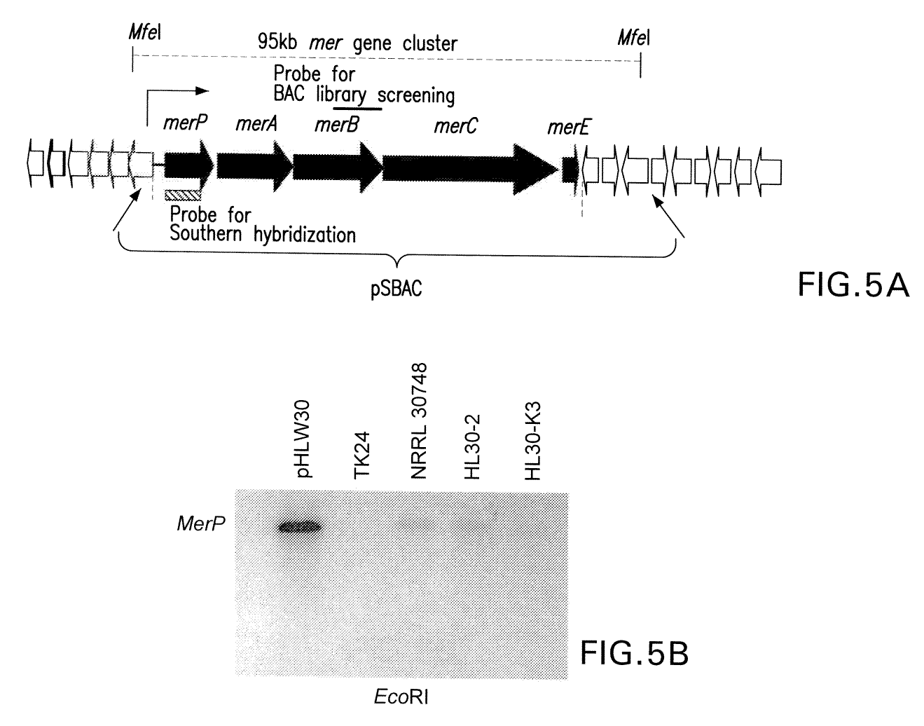

Previously, the meridamycin (mer) biosynthetic gene cluster from Streptomyces sp. NRRL 30748 was cloned into several cosmid clones and sequence results revealed that the genes that are responsible for the construction of the core structure of meridamycin were located in an approximately 90 kb of DNA fragment (He, M., Halti, B., Summers, M., Feng, X. and Hucul, J. (2006). Gene 377:109-118.). From the restriction digestion analysis of the sequence data, two restriction enzyme sites (Mfel) were found to be located at 378 bp upstream and ˜16 kb down stream of the mer gene cluster, respectively. This Mfel DNA fragment contains the DNA that covers the whole mer gene cluster with its original promoter and many downstream modification enzyme encoding regions (FIG. 5A). Southern analysis demonstrated the existence of the 97 kb band when the genomic DNA digested with Mfel and hybridized with mer gene cluster specific probe (data not shown). In order to clone this gene cluster, an Mfel-digested genomic BAC library was constructed using a E. coli-streptomycetes shuttle BAC vector, pSBAC which not only has the essential components of a BAC vector to accommodate large DNA inserts, but also contains an origin of transfer (oriT) which permits the transfer of the vector from E. coli to streptomycetes by conjugation and a φBT1 attP-int DNA fragment which encodes a site-specific recombination system that permit site-specific integration of the vector into the attB site of the recipient chromosome (Liu and He., Manuscript in preparation).

About 300 recombinant clones were screened by colony hybridization using the DNA probe that corresponds to the middle of the mer gene cluster. One positive clone, pHLW30 was selected for further analyses (SEQ ID NO: 31: mer gene cluster). End sequencing, restriction digestion and PCR amplification using primer sets corresponding to both ends of the mer gene cluster confirmed that the whole gene cluster has been successfully cloned into one single clone. This clone also contains the 378 bp upstream region of the gene cluster that possibly functions as the promoter and 16 kb downstream region that contains the genes that encode the potential tailoring enzymes.

Heterologous Expression of the Mer Gene Cluster in S. lividans.

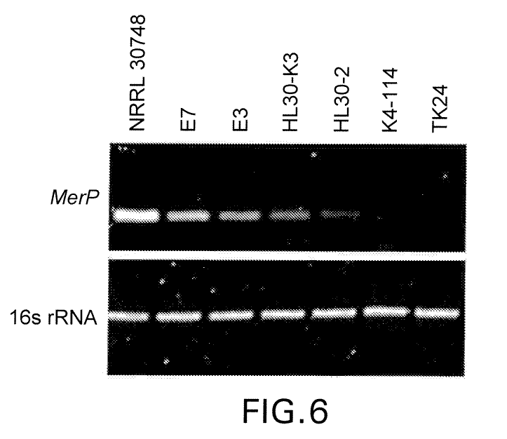

The BAC clone pHLW30 was transferred into S. lividans TK24 and K4-114 strains using the E. coli S17 via conjugation. The K4-114 strain is different from TK24 in that part of the act gene cluster in K4-114 had been deleted and abolished its ability to produce actinorhodin, therefore providing a cleaner background for heterologous expression. The Apramycin-resistant exconjugants were selected and PCR analyses using the primer sets that corresponding to both ends of the mer gene cluster confirmed the introduction of the gene cluster into the new host (data not shown). Southern analysis further confirmed the existence of the gene cluster in the new hosts, HL30-2 and HL30-K3 derived from TK24 and K4-114, respectively (FIG. 5B). In particular, plasmid pHLW30 and genomic DNA from different strains were digested with EcoRI, and then probed with merP specific DNA fragment. (pHL30-2: Mer gene cluster in TK24; pHL30-K3: Mer gene cluster in K4-114 (Act-strain)). Small-scale fermentation (25 ml) of strain HL30-2 and HL30-K3 in FKA fermentation media did not produce detectable amount of meridamycin by LC-MS analysis. As a first step to investigate the possible reasons that resulted in the failure of the heterologous host to produce the intended compound, we examined whether the original promoter of the mer gene cluster is active in the new hosts by analyzing the transcript of the gene cluster using RT-PCR. We were able to detect the transcript of this gene cluster from both hosts, suggesting the promoter is functional (data not shown). Further semi-quantitative RT-PCR revealed that the transcript level of mer gene cluster from the heterologous host was much lower than that of the original producer NRRL 30748 (FIG. 6), suggesting that the original mer promoter was not efficient in the new hosts and indicating transcriptional control might be the foremost factor that affects the production of meridamycin in the new hosts. The top panel of FIG. 6 represents the results of RT-PCR amplification from RNAs of various strains using primer set RT1 and RT2, which amplifies part of the MerP gene. The bottom panel of FIG. 6 represents the results of RT-PCR amplification from RNAs of corresponding strains using primer set 16sFor and 16sRev, which amplifies part of the 16sr rRNA. (TK24: S. lividans TK24, K4-114: S. lividans K4-114, HL30-2: mer gene cluster in S. lividans TK24, HL30-K3: Mer gene cluster in S. lividans K4-114, E3: Mer gene cluster with ermE* promoter in S. lividans TK24, E7: mer gene cluster with ermE* promoter in S. lividans K4-114). This result promoted us to change the original mer promoter with a constitutively active ermE* promoter which has been proven to be a strong promoter in S. lividans (Bibb, M. J., Janssen, G. R., and Ward, J. M. 1985. Gene 38:215-226). Plasmid pHLW71, which has the ermE* promoter in front of the 2 kb homologous sequence of beginning of the MerP gene, was used to replace the original mer promoter with ermE* promoter as a result of homologous recombination (FIG. 6). Numerous exconjugants were selected and PCR was used to select the strain that has the expected single cross-over homologous recombination. As a result of this recombination, the ermE* promoter was placed in front of the whole mer gene cluster and used to drive the transcription of it. Subsequent semi-quantitative RT-PCR demonstrated that changing promoters increased the transcription of mer gene cluster dramatically (FIG. 6). Although changing to ermE* promoter increased the transcription of mer gene cluster in S. lividans significantly (3 folds), the overall transcription of mer gene cluster in the new hosts was still lower than that in the original producer NRRL 30748 (FIG. 6). It is reasonable to postulate that the original meridamycin producing strain has a very efficient promoter to drive the expression of the gene cluster.

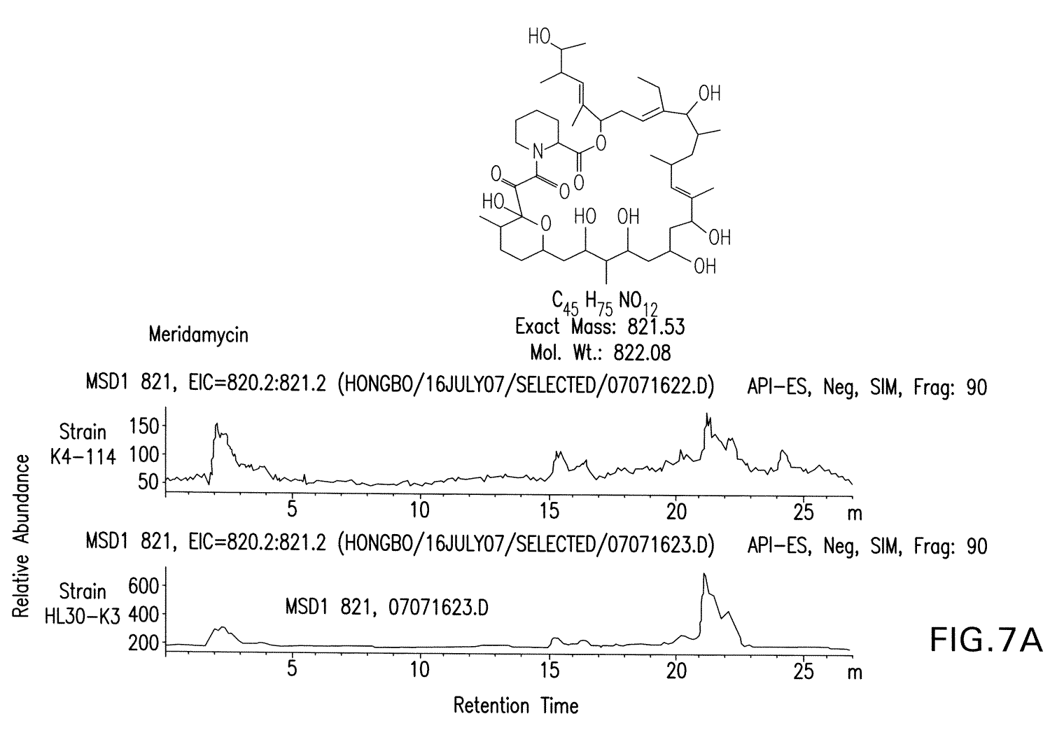

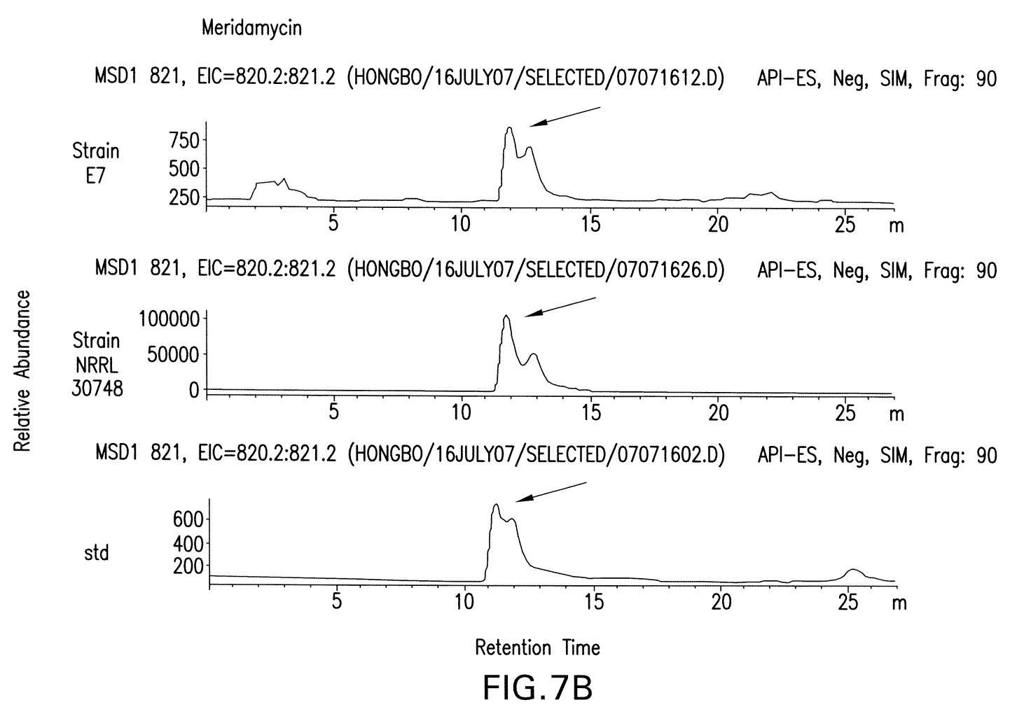

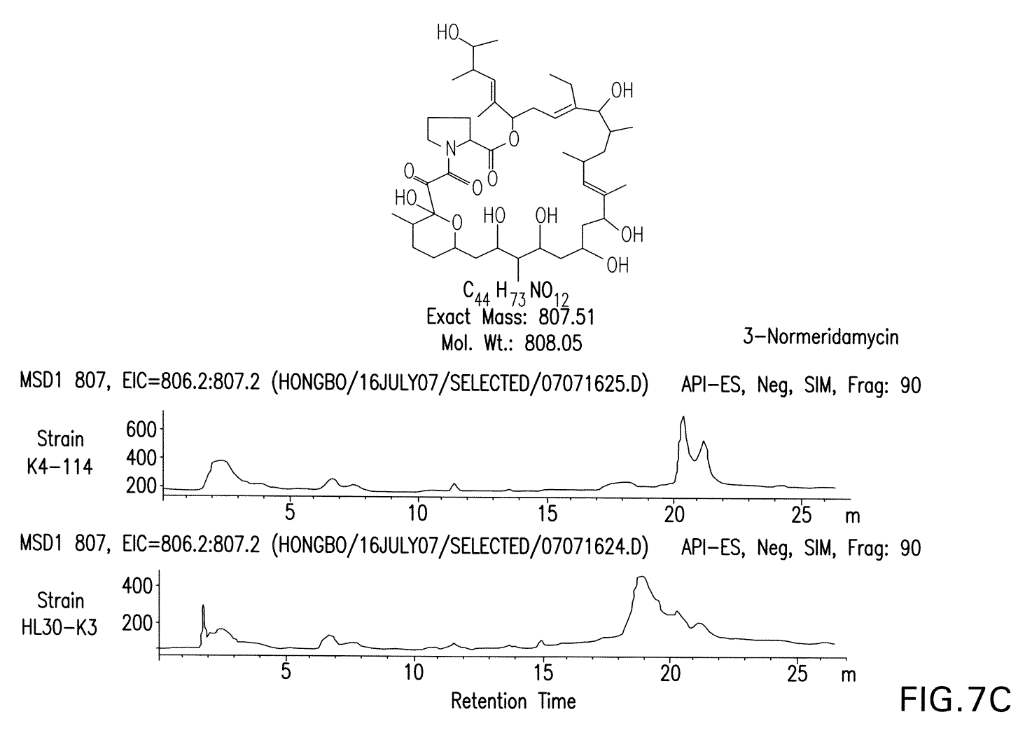

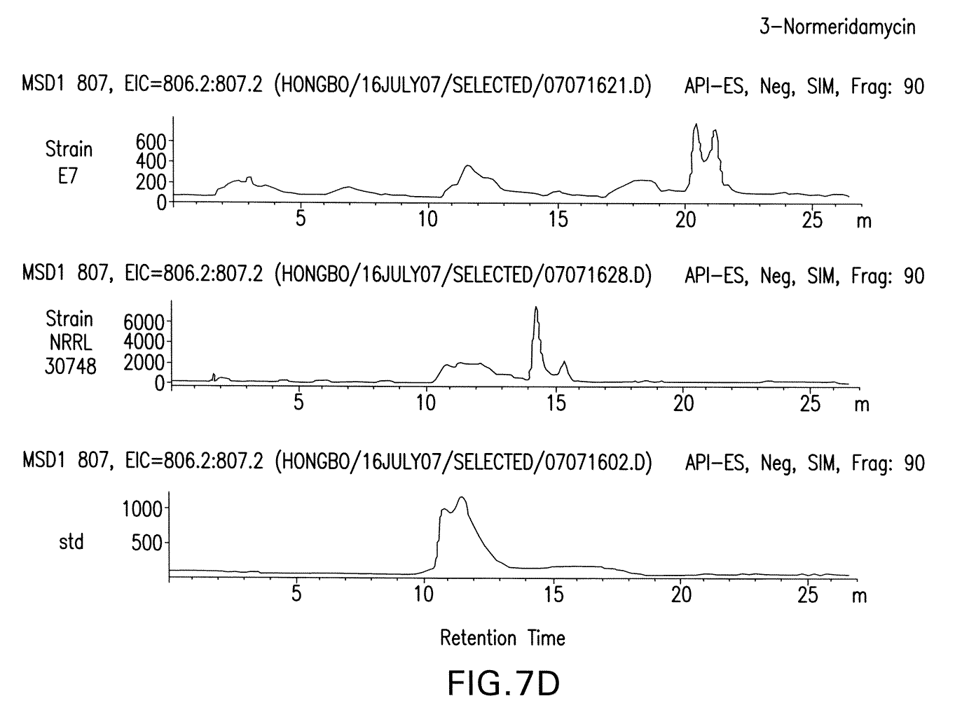

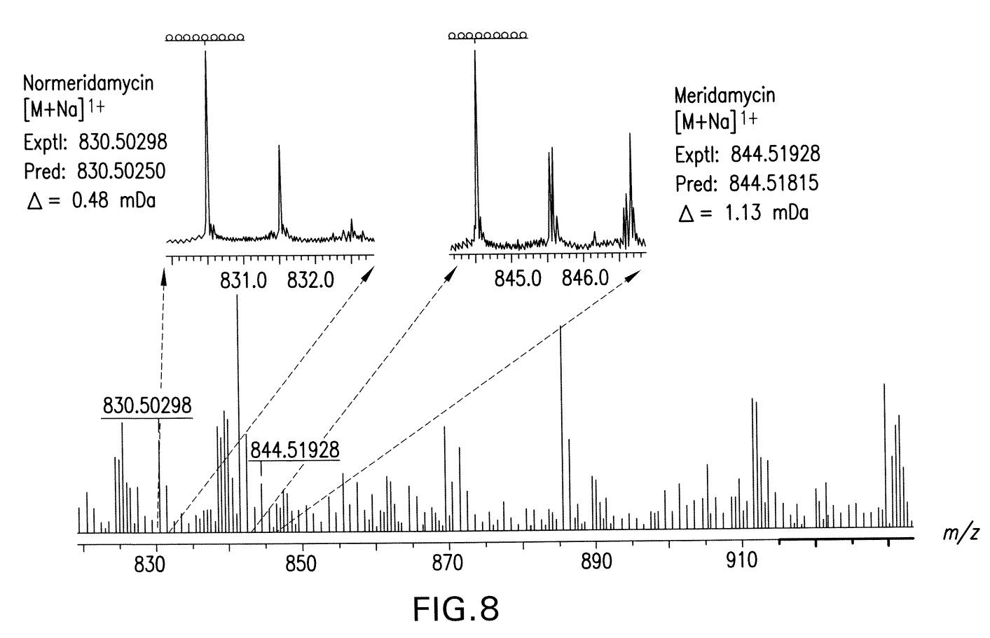

Subsequent LC-MS detection demonstrated the production of meridamycin from the strain E7 (FIG. 7), which has the original mer promoter changed with ermE* promoter in the S. lividans K4-114 background. The left panel of FIG. 7 shows the detection of the production of meridamycin from strains grown in FKA medium supplemented with 2 mM pipecolate and 10 mM diethymalonate. The right panel of FIG. 7 shows the detection of the production of 3-Normeridamycin from strains grown in FKA medium supplemented with 0.4% proline and 10 mM diethymalonate. The arrows indicate the peaks with expected molecular weight and retention time of either meridamycin or 3-Normeridamycin. The failure of the corresponding strain derived from S. lividans TK24 (strain HL30-2) to produce detectable amount of meridamycin may be due to precursor supply or to interference from the host metabolites (FIG. 7). The identity of the interested products was further confirmed by FTMS high-resolution accurate mass spectra using a nanoelectrospray source with long accumulation times. The sodium adduct molecular ion of meridamycin and normeridamycin with low abundance were detected in the positive ion mode at m/z 844.51928 and m/z 830.50298, respectively, and they agree very well with the predicted values ([M+Na]¹⁺, meridamycin: pred. 844.51815., Δ=1.13 mDa; Normeridamycin: pred. 830.50250., Δ=0.48 mDa), therefore confirmed the identity of the products to be meridamycin and 3-Normeridamycin (FIG. 8).

Previous analysis of each AT domain of the cloned meridamycin biosynthetic gene cluster revealed that three different extender units have to be presented for the complete synthesis of meridamycin, including malonate-CoA, methylmalonate-CoA and ethylmalonate-CoA (He, M., Halti, B., Summers, M., Feng, X. and Hucul, J. (2006), Gene 377:109-118). The acyltransferase (AT) in module 4 is responsible for the incorporation of ethylmalonyl-CoA into the polyketide backbone of meridamycin (He et al., 2006), but the gene cluster lacks the genes for biosynthesis of the eythimalonate extender unit and it has been suggested that other functional gene clusters may be able to synthesize ethymalonate and provide if for the production of meridamycin. Although S. lividans produces ethylmalonyl-CoA (Hu, Z., Reid, R., and Gramajo, H. 2005, J. Antibiot. 58:625-633.), this precursor has been demonstrated to be a critical factor that limits the synthesis of some polyketides by heterologous hosts (Jung, W. S., Lee, S. K., Hong, J. S. J., Park., S. R., Jeong, S. J., Han, A. R., Sohng, J. K., Kim B. G., Choi, C. Y., Sherman, D. H., and Yoon, Y. J. 2006, App. Microbiol. Biotech. 72:763-769.). To investigate whether the supply of ethymalonyl-CoA affects the production of meridamycin in the heterologous host, strain E7 was cultured in FKA media supplemented with 10 mM diethylmalonate, which has been proven to be an effective precursor for ethylmalonyl-CoA (Jung, W. S., Lee, S. K., Hong, J. S. J., Park., S. R., Jeong, S. J., Han, A. R., Sohng, J. K., Kim B. G., Choi, C. Y., Sherman, D. H., and Yoon, Y. J. 2006, App. Microbiol. Biotech. 72:763-769.). The result showed feeding with diethymalonate increased the production of meridamycin by 2 fold (from 150 μg/L to 300 μg/L).

One issue related to the original merdiamycin producing strain NRRL 30748 is the heterogeneity of the fermentation product, which is a mixture of meridamycin, 3-nor-meridamycin and C9-deoxomeridamycin. By heterologous expression the meridamycin biosynthetic gene cluster in S. lividans, 3-Normeridamycin was only detectable when proline was supplemented in the media from strain E7 (FIG. 8).

TABLE 2
Sequence Descriptions

| SEQ ID NO | Description |
|---|---|
| 1 | pSBAC vector |
| 2 | Ori 2 |
| 3 | Ori V |
| 4 | E. coli F factor partitioning system |
| 5 | Ori T |
| 6 | attB integration site in the Actinomycetes recipient cell |
| 7 | attP site in φBT1 |
| 8 | pCC1BAC vector |
| 9 | pCC1BAC forward primer |
| 10 | pCC1BAC reverse primer |
| 11 | pB1 forward primer |
| 12 | pB1 reverse primer |
| 13 | Scres 1 forward primer |
| 14 | Scres 2 reverse primer |
| 15 | pACT 1 forward primer |
| 16 | pACT 1 reverse primer |
| 17 | apramycin resistance gene from pBWA2 |
| 18 | attP integrase gene |
| 19 | RT1 primer |
| 20 | RT2 primer |
| 21 | 16s For primer |
| 22 | 16s Rev primer |
| 23 | P1 primer |
| 24 | P2 primer |
| 25 | OriTFOR primer |
| 26 | OriTREV primer |
| 27 | ErmE1 primer |
| 28 | ErmE2 primer |
| 29 | ErmE3 primer |
| 30 | ErmE4 primer |
| 31 | PHLW30 (whole mer cluster) DNA sequence |

What is claimed is:

1. A vector for cloning or transfer of a large DNA fragment comprising a whole, or a portion of a gene cluster, from one prokaryotic organism to a species of Actinomycetes, comprising
(a) at least two origins of replication;
(b) a prokaryotic F factor partitioning system;
(c) an origin of transfer;
(d) a site-specific recombination system that allows for the integration of the vector into the recipient cell; and
(e) a selection marker.

2. The vector of claim 1, further comprising a large DNA fragment.

3. The vector of claim 2, wherein the large DNA fragment comprises a whole or portion of a gene cluster.

4. A vector for cloning or transfer of a large DNA fragment comprising the whole, or a portion of a gene cluster, from one prokaryotic organism to a species of Actinomycetes, comprising:
(a) at least two origins of replication;
(b) a prokaryotic F factor partitioning system;
(c) an origin of transfer;
(d) a φBT1 attP-int recombination system; and
(e) a selection marker.

5. The vector of claim 4, further comprising a large DNA fragment.

6. The vector of claim 5, wherein the large DNA fragment comprises a whole or portion of a gene cluster.

7. The vector of either one of claims 1 or 4 wherein the prokaryotic organism is E. coli.

8. The vector of either one of claims 1 or 4 wherein the vector is a Bacterial Artificial Chromosome (BAC) vector.

9. The vector of claim 8, wherein the BAC vector is a shuttle BAC vector.

10. The vector of claim 9, wherein the shuttle BAC vector is an E. coli-Actinomycetes conjugative vector, pSBAC.

11. The vector of either one of claims 1 or 4, wherein the two origins of replication are E. coli origins of replication.

12. The vector of claim 11, wherein at least one of the origins of replication is selected from ori 2 and ori V.

13. The vector of claim 12, wherein at least one of the origins of replication comprises the nucleotide sequence of SEQ ID NO: 2 or SEQ ID NO: 3.

14. The vector of either one of claims 1 or 4, wherein the prokaryotic F factor partitioning system is an E. coli F factor partitioning system.

15. The E. coli F factor partitioning system of claim 14 comprising the nucleic acid sequence of SEQ ID NO: 4.

16. The vector of either one of claims 1 or 4, wherein the origin of transfer is oriT.

17. The vector of claim 16, wherein the origin of transfer comprises the nucleotide sequence of SEQ ID NO: 5.

18. The vector of either one of claims 3 or 6, wherein the gene cluster encodes one or more gene product(s) that are part of a specific biosynthetic pathway for secondary metabolites.

19. The vector of claim 18, wherein the gene cluster encodes the proteins that are involved in the biosynthesis of actinorhodin, meridamycin, or derivatives thereof.

20. The vector of claim 18, wherein the gene product(s) of the biosynthetic pathway for secondary metabolites is a polyketide or a non-ribosomal polypeptide (NRP).

21. The vector of claim 20, wherein the polyketide is selected from the group consisting of an antibiotic, an immunosuppressant, an anti-cancer agent, an anti-fungal agent and a cholesterol lowering agent.

22. A plasmid rescue method for isolating or cloning a large DNA fragment, wherein the large DNA fragment ranges in size from about 20 kb to about 100 kb, the method comprising:
(a) transferring any of the vectors of either one of claims 1 or 4 to a recipient Actinomycetes cell, which contains a nucleic acid having a site specific integration sequence that allows for the integration of the vector;
(b) selecting for the recipient Actinomycetes cell that contains the vector incorporated into the Actinomycetes chromosome;
(c) isolating the DNA from the chromosome of the recipient cell;
(d) transferring the DNA from step c) into an E. coli cell;
(e) screening for an E. coli cell that contains any of the vectors of claim 20; and
(f) isolating the large DNA fragment from the vector(s) of step e).

23. The method of claim 22, wherein the large DNA fragment comprises a whole or a portion of a gene cluster.

24. The method of claim 23, wherein the gene cluster encodes the proteins that are involved in the biosynthesis of actinorhodin, meridamycin, or derivatives thereof.

25. The method of claim 22, wherein the site specific integration sequence in the recipient cell is an att site.

26. The method of claim 22, wherein the att site in the recipient cell is an attB site comprising the nucleotide sequence of SEQ ID NO: 6.

27. A plasmid rescue method for isolating or cloning a large DNA fragment, wherein the DNA fragment ranges in size from about 20 kb to about 100 kb, the method comprising:
(a) transferring any of the vectors of either one of claims 1 or 4 to a recipient Actinomycetes cell, which contains a homologous sequence that allows for the homologous recombination of the vector;
(b) selecting for the recipient Actinomycetes cell that contains the vector incorporated into the Actinomycetes chromosome;
(c) isolating the DNA from the chromosome of the recipient cell;
(d) transferring the DNA from step c) into an E. coli cell;
(e) screening for an E. coli cell that contains any of the vectors of claim 20 and
(f) isolating the DNA fragment from the vector(s) of step e).

28. The method of claim 27, wherein the large DNA fragment is a whole or a portion of a gene cluster.

29. The method of claim 28, wherein the gene cluster encodes the proteins that are involved in the biosynthesis of actinorhodin, meridamycin, or derivatives thereof.

30. The method of either of claims 22 or 27, wherein the selecting step comprises selecting for a biological or enzymatic activity that is transferred to the recipient cell by the vector.

31. The method of either of claims 22 or 27, wherein the transferring of the vector comprises conjugating the donor cell containing the vector with a recipient Actinomycetes cell.

32. A method of producing meridamycin in actinomycetes comprising expressing the amino acids encoded by the mer gene cluster of SEQ ID NO: 31.

33. The method of claim 32, wherein the mer gene cluster is incorporated into the pSBAC vector of SEQ ID NO: 1.

34. The vector of claim 19, wherein the gene cluster comprises SEQ ID NO: 31.
